(19) United States
(12) Patent Application Publication
(10) Pub. No.: US 2026/0021205 A1
(43) Pub. Date: Jan. 22, 2026

(54) RECOMBINANT AAV HAVING AAV CLADE D AND CLADE E CAPSIDS AND COMPOSITIONS CONTAINING SAME

(71) Applicant: The Trustees of the University of Pennsylvania, Philadelphia, PA (US)

(72) Inventors: James M. Wilson, Philadelphia, PA (US); Qiang Wang, Philadelphia, PA (US); Kalyani Nambiar, New York, NY (US)

(73) Assignee: The Trustees of the University of Pennsylvania, Philadelphia, PA (US)

(21) Appl. No.: 18/994,948

(22) PCT Filed: Jul. 14, 2023
(86) PCT No.: PCT/US23/70218

§ 371 Date: Jan. 15, 2025

Related U.S. Application Data
(60) Provisional application No. 63/368,610, filed on Jul. 15, 2022.

(51) Int. Cl. A61K 48/00; C12N 15/86

(52) U.S. Cl. CPC A61K 48/0058; A61K 48/0041; C12N 15/86; C12N 2750/14122; C12N 2750/14143; C12N 2750/14152

(57) ABSTRACT

Novel AAV capsids and recombinant AAV vectors comprising the same are provided.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “UPN-22-10133.PCT_ST26_Sequence Listing.xml” (Created: Jul. 12, 2022; Size: 126,385 bytes).

### BACKGROUND OF THE INVENTION

Adeno-associated virus (AAV) vectors hold great promise in human gene therapy and have been widely used to target liver, muscle, heart, brain, eye, kidney, and other tissues in various studies due to their ability to provide long-term gene expression and lack of pathogenicity. Since the first trial which started in 1981, there has not been any vector-related toxicity reported in clinical trials of AAV vector-based gene therapy. The ever-accumulating safety records of AAV vector in clinical trials, combined with demonstrated efficacy, show that AAV is an attractive platform.

AAV belongs to the parvovirus family and contains a single-stranded DNA genome flanked by two inverted terminal repeats. AAV is easily manipulated as the virus has a single-stranded DNA virus with a relatively small genome (˜4.7 kb) and simple genetic components—inverted terminal repeats (ITR), the Rep and Cap genes. Only the ITRs and AAV capsid protein are required in AAV vectors, with the ITRs serving as replication and packaging signals for vector production and the capsid proteins playing a central role by forming capsids to accommodate vector genome DNA and determining tissue tropism. Dozens of naturally occurring AAV capsids have been reported; their unique capsid structures enable them to recognize and transduce different cell types and organs.

However, despite allowing for efficient gene transfer, the AAV vectors currently used in the clinic can be hindered by preexisting immunity to the virus and restricted tissue tropism. The isolation of adeno-associated virus (AAV) genomes from biomaterials at the molecular level has traditionally relied on polymerase chain reaction-based and cloning-based techniques. However, when applied to samples containing multiple species, traditional techniques for isolating viral genomes can amplify artificial recombinants and introduce polymerase misincorporation errors.

Thus, improved methods for isolating viral genomes and AAV capsid coding sequences are needed.

### SUMMARY OF THE INVENTION

In one aspect, provided herein is a recombinant adeno-associated virus (rAAV) comprising a capsid and a vector genome comprising an AAV 5′ inverted terminal repeat (ITR), an expression cassette comprising a nucleic acid sequence encoding a gene product operably linked to expression control sequences, and an AAV 3′ ITR, wherein the capsid comprises: (a) (i) an AAVrh94 capsid produced by expressing a nucleic acid sequence encoding the AAVrh94 VP1 of SEQ ID NO: 10, (ii) an AAVrh94 capsid produced by expressing SEQ ID NO: 9 or a sequence at least 99% identical thereto encoding SEQ ID NO: 10; or (iii) at least AAV rh94 VP1 and VP3 proteins which are 95% to 100% deamidated in at least position N57, N263, N384, and/or N514 based on the residue position numbers of SEQ ID NO: 10, and optionally deamidated in other positions; (b) (i) an AAVrh95 capsid produced by expressing a nucleic acid sequence encoding the AAVrh95 VP1 of SEQ ID NO: 12, (ii) an AAVrh95 capsid produced by expressing SEQ ID NO: 11 or a sequence at least 99% identical thereto encoding SEQ ID NO: 12; or (iii) at least AAV rh95 VP1 and VP3 proteins which are 95% to 100% deamidated in at least position N57, N384, and/or N514 based on the residue position numbers of SEQ ID NO: 12, and optionally deamidated in other positions; (c) (i) an AAVrh96 capsid produced by expressing SEQ ID NO: 13 or a sequence at least 98% identical thereto; (d) (i) an AAVrh97 capsid produced by expressing a nucleic acid sequence encoding the AAVrh97 VP1 of SEQ ID NO: 16, (ii) an AAVrh97 capsid produced by expressing SEQ ID NO: 15 or a sequence at least 99% identical thereto encoding SEQ ID NO: 16; or (iii) at least AAV rh97 VP1 and VP3 proteins which are 95% to 100% deamidated in at least position N57, N263, N384, and/or N514 based on the residue position numbers of SEQ ID NO: 16, and optionally deamidated in other positions; (e) (i) an AAVrh98 capsid produced by expressing a nucleic acid sequence encoding the AAVrh98 VP1 of SEQ ID NO: 18, (ii) an AAVrh98 capsid produced by expressing SEQ ID NO: 17 or a sequence at least 99% identical thereto encoding SEQ ID NO: 28; or (iii) at least AAVrh98 VP1 and VP3 proteins which are 95% to 100% deamidated in at least position N57, N263, N384, and/or N514 based on the residue position of SEQ ID NO: 18, and optionally deamidated in other positions; or (f) (i) an AAVrh99 capsid produced by expressing a nucleic acid sequence encoding the AAVrh99 VP1 of SEQ ID NO: 20, (ii) an AAVrh99 capsid produced by expressing SEQ ID NO: 19 or a sequence at least 99% identical thereto encoding SEQ ID NO: 20; or (iii) at least AAV rh99 VP1 and VP3 proteins which are 95% to 100% deamidated in at least position N57, N263, N384, and/or N515 based on the residue positions of SEQ ID NO: 20, and optionally deamidated in other positions.

In certain embodiments, the rAAV further comprises: (a) AAVrh94 VP3 proteins having the amino acid sequence of about residue 204 to about 728 of SEQ ID NO: 10 (SEQ ID NO: 34) which are 95% to 100% deamidated in at least position N57, N263, N384, and/or N514 based on the residue position numbers of SEQ ID NO: 10, and optionally deamidated in other positions; (b) AAVrh95 VP3 proteins having the amino acid sequence of about residue 212 to about 737 of SEQ ID NO: 12 (SEQ ID NO: 35) which are 95% to 100% deamidated in at least position N57, N384, and/or N514 based on the residue position numbers of SEQ ID NO: 12, and optionally deamidated in other positions; (c) AAVrh97 VP3 proteins having the amino acid sequence of about residue 204 to about 739 of SEQ ID NO: 12 (SEQ ID NO: 36) which are 95% to 100% deamidated in at least position N57, N263, N384, and/or N514 based on the residue position numbers of SEQ ID NO: 16, and optionally deamidated in other positions; (d) AAVrh98 VP3 proteins having the amino acid sequence of about residue 204 to about 738 of SEQ ID NO: 16 (SEQ ID NO: 37) which are 95% to 100% deamidated in at least position N57, N263, N384, and/or N514 based on the residue position of SEQ ID NO: 16, and optionally deamidated in other positions; or (e) AAVrh99 VP3 proteins having the amino acid sequence of about residue 212 to about 738 of SEQ ID NO: 20 (SEQ ID NO: 37) which are 95% to 100% deamidated in at least position N57, N263, N384, and/or N515 based on the residue positions of SEQ ID NO: 20, and optionally deamidated in other positions.

In certain embodiments, the rAAV comprises a sequence encoding a gene product useful in treating a disorder or disease of the liver. In certain embodiments, the rAAV comprises a sequence encoding a gene editing nuclease. In certain embodiments, the rAAV comprises a constitutive promoter. In certain embodiments, the rAAV comprises a tissue-specific promoter.

In certain embodiments, a host cell is provided which comprises a rAAV having an AAVrh94, AAVrh94, AAVrh96, AAVrh97, AAVrh98 or AAVrh99 capsid.

In certain embodiments, a pharmaceutical composition comprises an rAAV as provided herein and a physiologically compatible carrier, buffer, adjuvant, and/or diluent.

In certain embodiments, a method for delivering a transgene to a cell is provided which comprises administering an rAAV or/or composition as provided herein which comprises the transgene.

In certain embodiments, a plasmid comprising a AAV vp1 capsid nucleic acid sequence is provided which comprises: (i) an AAVrh94 nucleic acid sequence encoding the AAVrh94 VP1 of SEQ ID NO: 10 or an AAVrh94 capsid produced by expressing SEQ ID NO: 9 or a sequence at least 99% identical thereto encoding SEQ ID NO: 10; (ii) an AAVrh95 nucleic acid sequence encoding the AAVrh95 VP1 of SEQ ID NO: 12 or an AAVrh95 capsid produced by expressing SEQ ID NO: 11 or a sequence at least 99% identical thereto encoding SEQ ID NO: 12; (iii) an AAVrh96 nucleic acid sequence produced by expressing SEQ ID NO: 13 or a sequence at least 98% identical thereto; (iv) an AAVrh97 nucleic acid sequence encoding the AAVrh97 VP1 of SEQ ID NO: 16 or an AAVrh97 capsid produced by expressing SEQ ID NO: 15 or a sequence at least 99% identical thereto encoding SEQ ID NO: 16; (v) an AAVrh98 nucleic acid sequence encoding the AAVrh98 VP1 of SEQ ID NO: 18 or (ii) an AAVrh99 capsid produced by expressing SEQ ID NO: 17 or a sequence at least 99% identical thereto encoding SEQ ID NO: 18; or (vi) an AAVrh99 capsid nucleic acid sequence encoding the AAVrh99 VP1 of SEQ ID NO: 20 or an AAVrh99 capsid produced by expressing SEQ ID NO: 19 or a sequence at least 99% identical thereto encoding SEQ ID NO: 20.

In certain embodiments, a method is provided for generating a recombinant adeno-associated virus (rAAV) comprising an AAV capsid. The method may comprise culturing a host cell containing: (a) a molecule encoding an AAV vp1, vp2, and/or vp3 capsid protein of: (i) an AAVrh94 capsid produced by expressing a nucleic acid sequence encoding the AAVrh94 VP1 of SEQ ID NO: 10 or an AAVrh94 capsid produced by expressing SEQ ID NO: 9 or a sequence at least 99% identical thereto encoding SEQ ID NO: 10; (ii) an AAVrh95 capsid produced by expressing a nucleic acid sequence encoding the AAVrh95 VP1 of SEQ ID NO: 12 or an AAVrh95 capsid produced by expressing SEQ ID NO: 11 or a sequence at least 99% identical thereto encoding SEQ ID NO: 12; (iii) an AAVrh96 capsid produced by expressing SEQ ID NO: 13 or a sequence at least 98% identical thereto; (iv) an AAVrh97 capsid produced by expressing a nucleic acid sequence encoding the AAVrh97 VP1 of SEQ ID NO: 16 or (ii) an AAVrh97 capsid produced by expressing SEQ ID NO: 15 or a sequence at least 99% identical thereto encoding SEQ ID NO: 27; (v) an AAVrh98 capsid produced by expressing a nucleic acid sequence encoding the AAVrh98 VP1 of SEQ ID NO: 18 or (ii) an AAVrh98 capsid produced by expressing SEQ ID NO: 17 or a sequence at least 99% identical thereto encoding SEQ ID NO: 18; or (vi) an AAVrh99 capsid produced by expressing a nucleic acid sequence encoding the AAVrh99 VP1 of SEQ ID NO: 20 or an AAVrh99 capsid produced by expressing SEQ ID NO: 19 or a sequence at least 99% identical thereto encoding SEQ ID NO: 20, (b) a functional rep gene; (c) a vector genome comprising an AAV 5′ inverted terminal repeats (ITR), a transgene operably linked to expression control sequences, and an AAV 3′ ITR; and (d) sufficient helper functions to permit packaging of the vector genome into the AAV capsid protein.

In certain embodiments, a host cell in culture or suspension which comprises a nucleic acid molecule encoding the AAVrh94, AAVrh95, AAVrh96, AAVrh97, AAVrh98, or AAVrh99 capsid.

In one aspect, provided herein is a pharmaceutical composition comprising a rAAV, and a physiologically compatible carrier, buffer, adjuvant, and/or diluent.

Other aspects and advantages of these compositions and methods are described further in the following detailed description.

### BRIEF DESCRIPTION OF THE DRAWINGS

FIGS. 1A to 1E illustrate schematic of the AAV-SGA procedure. (FIG. 1A) We screened bulk mammalian gDNA samples by PCR using AAV-specific primers that amplify a 3.1-kb region of the AAV genome encompassing the terminal third of the Rep gene and the complete Cap gene sequence. (FIGS. 1B to FIG. 1C) A sample yielding positive results for AAV-detection PCR is endpoint-diluted in a 96-well plate format and used as the template for 3.1-kb amplicon AAV-specific PCR. A gDNA dilution with less than a 30% positive PCR rate contains one amplifiable AAV genome in each reaction. (FIG. 1D) Each positive amplicon was size-selected and sequenced using the Illumina MiSeq platform. (FIG. 1E) Reads originating from single genomes were de novo assembled to recover full-length AAV contigs containing the VP1 capsid gene. AAV: adeno-associated virus; gDNA: genomic DNA; NGS: next-generation sequencing; PCR: polymerase chain reaction; SGA: single-genome amplification.

FIGS. 2A to 2D. Precise recovery of AAV amplicons via AAV-SGA. (FIG. 2A) We utilized AAV-SGA to isolate single full-length AAV capsid sequences from a mixture of four AAV trans plasmids. (FIG. 2B) AAV-SGA virus distribution of novel AAV isolates AAVrh76 and AAVrh77 originating from NHP1 gDNA (left). Gel image depicting bulk PCR of AAV-containing gDNA from a macaque small intestine tissue sample, NHP1 (right). Comparison of aligned amplicons recovered from AAV-SGA (FIG. 2C) and bulk PCR (FIG. 2D) from identical source gDNA. Amplicons are aligned to the AAVrh76 reference sequence, and black bars denote sequence variations from the reference. Amplicon names are colored based on the AAV sequences: AAVrh76 (black), AAVrh77, AAVrh76-AAVrh77 hybrids. Amplicons were trimmed at the 3′ end for clarity.

FIGS. 3A to 3D illustrate the use of AAV-SGA to isolate novel AAV genome sequences from rhesus macaque liver samples. (FIG. 3A) Agarose gel of bulk AAV PCR results from liver samples. (FIGS. 3B and FIG. 3C) Distribution of novel AAV genomes recovered by AAV-SGA from rhesus macaque liver tissue samples. (FIG. 3D) Maximum-likelihood phylogenetic tree of newly isolated AAV-SGA variants and other prototypical AAVs to represent all known major clades: AAV1-AAV9, AAVrh32.33, and AAVrh10. The scale bar denotes 0.2 base substitutions per site. The circled branch nodes represent bootstrap support values >75, as determined by approximate likelihood ratio tests.

FIGS. 4A to 4D Recombination of AAV genomes in the caudate liver lobe of NHP2. (FIG. 4A) Schematic of unique AAV genomes isolated from tissue of the caudate lobe and regions of predicted recombination events. Neighbor-joining phylogenies of partial sequences from predicted recombination regions for events 1 (FIG. 4B) (p=1.239e-40) and 2 (FIG. 4C) (p=1.515e-11). (FIG. 4D) Neighbor-joining phylogeny of full-length sequences of unique AAVs isolated from the caudate lobe. The circled branch nodes represent bootstrap support values >75.

FIG. 5A to FIG. 5F show recombination of AAV genomes in the right liver lobe of NHP2. (FIG. 5A) Schematic of unique AAV genomes isolated from tissue of the caudate lobe and regions of predicted recombination events. Neighbor-joining phylogenies of partial sequences from the predicted recombination regions for events 1 (FIG. 5B) (p=7.226e-12), 2 (FIG. 5C) (p=1.794e-32), 3 (FIG. 5D) (p=2.167e-6), and 4 (FIG. 5E) (p=1.870e-29). (F) Neighbor-joining phylogeny of full-length sequences of unique AAVs isolated from the caudate lobe. The circled branch nodes represent bootstrap support values >75.

FIGS. 6A to 6D provide a Phylogenetic analysis of AAV natural isolate sequences. We used aligned amino acid sequences of the AAV VP1 gene to construct a neighbor-joining phylogenetic tree of AAV natural isolates available on NCBI GenBank and novel AAV-SGA isolates. The scale bar denotes 0.06 base substitutions per site. The clade nomenclature is displayed on the right, with major clade members shown.

FIGS. 7A to 7C provide an alignment of the encoded amino acid sequences of the VP1 proteins of the Clade D capsids identified herein, AAVrh94 (SEQ ID NO: 10) and AAVrh95 (SEQ ID NO: 12), aligned with previously published AAV1 VP1 capsid proteins, AAVrh85 (SEQ ID NO:45) and AAV7 (SEQ ID NO: 46).

FIGS. 8A to 8D provide an alignment of the encoded amino acid sequences of the VP1 proteins of the Clade E capsids identified herein, AAVrh97 (SEQ ID NO: 14), AAVrh98 (SEQ ID NO: 18), and AAVrh99 (SEQ ID NO: 20), aligned with previously published AAV1 VP1 capsid proteins, AAVrh64R1 (SEQ ID NO:41) and AAVrh46 (SEQ ID NO:47).

### DETAILED DESCRIPTION OF THE INVENTION

AAV single-genome amplification (AAV-SGA): a powerful technique to isolate, amplify, and sequence single AAV genomes from mammalian genomic DNA, which are then be used to construct vectors for gene therapy. We used AAV-SGA to precisely isolate novel AAV genomes belonging AAV clades A, D, and E and the Fringe outgroup. This technique also enables investigations of AAV population dynamics and recombination events to provide insights into virus-host interactions and virus biology. Using AAV-SGA, we identified regional heterogeneity within AAV populations from different lobes of the liver of a rhesus macaque and found evidence of frequent genomic recombination between AAV populations. This study highlights the strengths of AAV-SGA and demonstrates its capability to provide valuable insights into the biology and diversity of AAVs.

Unless defined otherwise, technical and scientific terms used herein have the same meaning as commonly understood by one of ordinary skill in the art to which this invention belongs and by reference to published texts, which provide one skilled in the art with a general guide to many of the terms used in the present application. The following definitions are provided for clarity only and are not intended to limit the claimed invention.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. Preferably, the homology is over full-length sequence, or an open reading frame thereof, or another suitable fragment which is at least 15 nucleotides in length. Examples of suitable fragments are described herein.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. Preferably, the homology is over full-length sequence, or a protein thereof, e.g., a cap protein, a rep protein, or a fragment thereof which is at least 8 amino acids, or more desirably, at least 15 amino acids in length. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”. “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. In the examples, AAV alignments are performed using the published AAV9 sequences as a reference point. Alignments are performed using any of a variety of publicly or commercially available Multiple Sequence Alignment Programs. Examples of such programs include, “Clustal Omega”, “Clustal W”, “MUSCLE”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 10.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 10.1, herein incorporated by reference. Multiple sequence alignment programs are also available for amino acid sequences, e.g., the “Clustal Omega”, “Clustal X”, “MUSCLE”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product.

A “genetic element” includes any nucleic acid molecule, e.g., naked DNA, a plasmid, phage, transposon, cosmid, episome, virus, etc., which transfers the sequences carried thereon. Optionally, such a genetic element may utilize a lipid-based carrier. Unless otherwise specified, the genetic element may be delivered by any suitable method, including transfection, electroporation, liposome delivery, membrane fusion techniques, high velocity DNA-coated pellets, viral infection and protoplast fusion.

A “stable host cell” for rAAV production is a host cell with had been engineered to contain one or more of the required rAAV production elements (e.g., minigene, rep sequences, the AAVhu68 engineered cap sequences as defined herein, and/or helper functions) and its progeny. A stable host cell may contain the required component(s) under the control of an inducible promoter. Alternatively, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from HEK293 cells (which contain E1 helper functions under the control of a constitutive promoter), Huh7 cells, Vero cells, engineered to contain helper functions under the control of a suitable promoter, which optionally further contains the rep and/or cap proteins under the control of inducible promoters. Still other stable host cells may be generated by one of skill in the art.

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product.

The abbreviation “se” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

As used herein, the term “operably linked” refers to both expression control sequences that are contiguous with the gene of interest and expression control sequences that act in trans or at a distance to control the gene of interest.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

In many instances, rAAV particles are referred to as DNase resistant. However, in addition to this endonuclease (DNase), other endo- and exo-nucleases may also be used in the purification steps described herein, to remove contaminating nucleic acids. Such nucleases may be selected to degrade single stranded DNA and/or double-stranded DNA, and RNA. Such steps may contain a single nuclease, or mixtures of nucleases directed to different targets, and may be endonucleases or exonucleases.

The term “nuclease-resistant” indicates that the AAV capsid has fully assembled around the expression cassette which is designed to deliver a gene to a host cell and protects these packaged genomic sequences from degradation (digestion) during nuclease incubation steps designed to remove contaminating nucleic acids which may be present from the production process.

As used herein, an “effective amount” refers to the amount of the rAAV composition which delivers and expresses in the target cells an amount of the gene product from the vector genome. An effective amount may be determined based on an animal model, rather than a human patient. Examples of a suitable murine model are described herein.

The term “translation” in the context of the present invention relates to a process at the ribosome, wherein an mRNA strand controls the assembly of an amino acid sequence to generate a protein or a peptide.

As used herein, the terms “a” or “an”, refers to one or more, for example, “an expression cassette” is understood to represent one or more expression cassettes. As such, the terms “a” (or “an”), “one or more,” and “at least one” are used interchangeably herein.

As used herein, the term “about” means a variability of 10% from the reference given, unless otherwise specified.

While various embodiments in the specification are presented using “comprising” language, under other circumstances, a related embodiment is also intended to be interpreted and described using “consisting of” or “consisting essentially of” language.

With regard to the following description, it is intended that each of the compositions herein described, is useful, in another embodiment, in the methods of the invention. In addition, it is also intended that each of the compositions described as useful in the methods, is, in another embodiment, itself an embodiment of the invention.

### A. The AAV Capsid

Nucleic acids encoding AAV capsids include three overlapping coding sequences, which vary in length due to alternative start codon usage. The translated proteins are referred to as VP1, VP2 and VP3, with VPT being the longest and VP3 being the shortest. The AAV particle consists of all three capsid proteins at a ratio of ˜1:1:10 (VP1:VP2:VP3). VP3, which is comprised in VP1 and VP2 at the N-terminus, is the main structural component that builds the particle. The capsid protein can be referred to using several different numbering systems. For convenience, as used herein, the AAV sequences are referred to using VP1 numbering, which starts with aa 1 for the first residue of VP1. However, the capsid proteins described herein include VP1, VP2, and VP3 (used interchangeably herein with vp1, vp2, and vp3).

| | | Protein/ | |
|---|---|---|---|
| AA/SEQ ID NO: | BLASTn 5/27 (DNA) | SEQ ID NO: | Blastp |
| >AAVrh90_VP1 | AAVrh.64, AY530574, | >AAVrh90_VP1 | AAVrh.64R1 |
| [SEQ ID NO: 1] | 2136/2217(96%) | [SEQ ID NO: 2] | ACB55316 |
| [Clade E] | | | (reproduced in SEQ |
| | | | ID NO: 41) |
| | | | 733/738(99%) |
| >AAVrh91_VP1 | AAV VR-355, | >AAVrh91_VP1 | AAV6.1 ACB55301 |
| [SEQ ID NO: 3] | DQ180605, | [SEQ ID NO: 4] | (reproduced in SEQ |
| [Clade A] | 2102/2211(95%) | | ID NO: 42) |
| | | | 730/736(99%) |
| >AAVrh92_VP1 | AAVrh77, MZ668385, | >AAVrh92_VP1 | AAVrh77 QZX63264 |
| [SEQ ID NO: 5] | 2175/2199(99%) | [SEQ ID NO: 6] | (reproduced in SEQ |
| [Fringe] | | | ID NO: 43) |
| | | | 730/733(99%) |
| >AAVrh93_VP1 | AAVrh89, MZ668394, | >AAVrh93_VP1 | AAVrh89 QZX63273 |
| [SEQ ID NO: 7] | 2113/2187(97%) | [SEQ ID NO: 8] | (reproduced in SEQ |
| [Clade D] | | | ID NO: 44) |
| | | | 720/728(99%) |
| >AAVrh94_VP1 | AAVrh85, MZ668392, | >AAVrh94_VP1 | AAVrh85 QZX63271 |
| [SEQ ID NO: 9] | 2180/2214(98%) | [SEQ ID NO: 10] | (reproduced in SEQ |
| [Clade D] | | | ID NO: 45) |
| | | | 734/737(99%) |
| >AAVrh95_VP1 | AAV7, AF513851, | >AAVrh95_VP1 | AAV7 YP_077178 |
| [SEQ ID NO: 11] | 2166/2214(98%) | [SEQ ID NO: 12] | (reproduced in SEQ |
| [Clade D] | | | ID NO: 46) |
| | | | 735/737(99%) |
| >AAVrh96_VP1 | AAVrh.64, AY530574, | >AAVrh96_VP1 | n/a (having one-bp- |
| [SEQ ID NO: 13] | Identities | [SEQ ID NO: 14] | insertion in the |
| [Clade E] | 2147/2218(97%), Gaps | | middle of the VP1) |
| | 1/2218(0%) | | |
| >AAVrh97_VP1 | AAVrh.64, AY530574, | >AAVrh97_VP1 | AAVrh.64R1 |
| [SEQ ID NO: 15] | 2164/2217(98%) | [SEQ ID NO: 16] | ACB55316 |
| [Clade E] | | | 736/738(99%) |
| >AAVrh98_VP1 | AAVrh.64, AY530574, | >AAVrh98_VP1 | AAVrh.64R1 |
| [SEQ ID NO: 17] | 2163/2217(98%) | [SEQ ID NO: 18] | ACB55316 |
| [Clade E] | | | 734/738(99%) |
| >AAVrh99_VP1 | AAVrh83, MZ668390, | >AAVrh99_VP1 | AAVrh.46 |
| [SEQ ID NO: 19] | Identities | [SEQ ID NO: 20] | ACB55314 |
| [Clade E] | 2109/2220(95%), Gaps | | 712/739(96%), Gaps |
| | 6/2220(0%) | | 1/739(0%) |

| | | Protein/ | |
|---|---|---|---|
| AA/SEQ ID NO: | VP3 start | SEQ ID NO: | Blastp |
| >AAVrh90_VP3 | 204-738 of SEQ | >AAVrh90_VP3 | AAVrh.64R1 ACB55316.1 |
| [SEQ ID NO: 21] | ID NO: 21 | [SEQ ID NO: | 532/535(99%) |
| | | 30] | |
| >AAVrh91_VP1 | 203-736 of SEQ | >AAVrh91_VP3 | AAV6.1 ACB55301 532/534(99%) |
| [SEQ ID NO: 22] | ID NO: 22 | [SEQ ID NO: | |
| | | 31] | |
| >AAVrh92_VP1 | 197-733 of SEQ | >AAVrh92_VP3 | AAVrh32.33 ACB55318.1 |
| [SEQ ID NO: 23] | ID NO: 23 | [SEQ ID NO: | (reproduced in SEQ ID NO: 50) |
| | | 32] | 535/537(99%) |
| >AAVrh93_VP1 | 207-728 of SEQ | >AAVrh93_VP3 | AAVrh89 QZX63273.1 |
| [SEQ ID NO: 24] | ID NO: 24 | [SEQ ID NO: | (reproduced in SEQ ID NO: 51) |
| | | 33] | 519/522(99%) |
| >AAVrh94_VP1 | 204-737 of SEQ | >AAVrh94_VP3 | AAVrh85 (reproduced in SEQ ID |
| [SEQ ID NO: 25] | ID NO: 25 (nt | [SEQ ID NO: | NO: 52) QZX63271.1 |
| | 610 to 2214 of | 34] | 534/534(100%) |
| | SEQ ID NO: 9) | | |
| >AAVrh95_VP1 | 212-737 of SEQ | >AAVrh95_VP3 | AAV7 YP_077178.1 |
| [SEQ ID NO: 26] | ID NO: 26 (nt | [SEQ ID NO: | 525/526(99%) |
| | 634 to 2214 of | 35] | |
| | SEQ ID NO: 11) | | |
| >AAVrh96_VP1 | | >AAVrh96_VP3 | n/a |
| >AAVrh97_VP1 | 204-739 of | >AAVrh97_VP3 | AAVrh.64R1 ACB55316.1 |
| [SEQ ID NO: 27] | SEQ ID NO: 27 | [SEQ ID NO: | 534/535(99%) |
| | (nt 610 to 2217 | 36] | |
| | of SEQ ID NO: | | |
| | 13) | | |
| >AAVrh98_VP1 | 204-738 of | >AAVrh98_VP3 | AAVrh.64R1 ACB55316.1 |
| [SEQ ID NO: 28] | SEQ ID NO: 28 | [SEQ ID NO: | 532/535(99%) |
| | (nt 610 to 2217 | 37] | |
| | of SEQ ID NO: | | |
| | 15) | | |
| >AAVrh99_VP1 | 212-738 of | >AAVrh99_VP3 | Anc84 AKU89599.1 (Reproduced |
| [SEQ ID NO: 29] | SEQ ID NO: 29 | [SEQ ID NO: | in SEQ ID NO: 54) 507/528(96%) |
| | (nt 634 to 2220 | 38] | |
| | of SEQ ID NO: | | |
| | 17) | | |

### Clade D

Provided herein are novel AAV capsid proteins having vp1 sequences: AAVrh94 and AAVrh95. The numbering of the nucleotides and amino acids corresponding to the vp1 and vp3 proteins are provided in the Tables above.

In certain embodiments, provided herein are rAAV comprising at least one of the vp1, vp2, and vp3 of AAVrh94 or AAVrh95. A recombinant adeno-associated virus (rAAV) comprising a capsid and a vector genome comprising an AAV 5′ inverted terminal repeat (ITR), an expression cassette comprising a nucleic acid sequence encoding a gene product operably linked to expression control sequences, and an AAV 3′ ITR, wherein the rAAV capsid is AAVrh94 or AAVrh95. In certain embodiments, the AAVrh94 capsid is produced by expressing a nucleic acid sequence encoding the AAVrh94 VP1 of SEQ ID NO: 10, (ii) an AAVrh94 capsid produced by expressing SEQ ID NO: 9 or a sequence at least 99% identical thereto encoding SEQ ID NO: 10; or (iii) at least AAV rh94 VP1 and VP3 proteins which are highly deamidated in at least four positions. In certain embodiments, the capsids are 75% to 100% deamidated in position N57, N263, N384, and/or N515 based on the residue position numbers of SEQ ID NO: 10, and optionally deamidated in other positions.

In certain embodiments, an rAAVrh95 capsid is produced by expressing a nucleic acid sequence encoding the AAVrh95 VP1 of SEQ ID NO: 12, (ii) an AAVrh95 capsid produced by expressing SEQ ID NO: 11 or a sequence at least 99% identical thereto encoding SEQ ID NO: 12; or (iii) at least AAV rh95 VP1 and VP3 proteins which are highly deamidated in at least four positions. In certain embodiments, the capsid are 75% to 100%, or at least 95% to 100% deamidated in position N57, N263, N384, and/or N515 based on the residue position numbers of SEQ ID NO: 12, and optionally deamidated in other positions.

In certain embodiments, rAAV having a capsid protein comprising a vp1, vp2, and/or vp3 sequence at least 99% identical to SEQ ID NO: 10 or 12 are provided. In certain embodiments, the vp1, vp2, and/or has up to 1, up to 2, up to 3, up to 4, up to 5, up to 6, up to 7, up to 8, up to 9, or up to 10 amino acid differences relative to the vp1, vp2, and/or vp3 of SEQ ID NO: 10 or 12. Also provided herein are rAAV comprising AAV capsids encoded by at least one of the vp1, vp2, and the vp3 sequence of any of SEQ ID NO: 25 or 26 or a sequence at least 99% identical to SEQ ID NO: 25 or 26. In certain embodiments, the sequence encodes a full-length vp1, vp2 and/or vp3 of SEQ ID NO: 10 or 12. In other embodiments, the vp1, vp2 and/or vp3 has an N-terminal and/or a C-terminal truncation (e.g. truncation(s) of about 1 to about 10 amino acids).

### Clade E

Provided herein are novel AAVrh96, AAVrh97, AAV97, AAV98 and AAV99 capsid proteins having the vp1 sequences set forth in the preceding tables.

In certain embodiments, an rAAV is provided which has an AAVrh96 capsid. The rAAV96 capsid may be produced by expressing SEQ ID NO: 13 or a sequence at least 98% identical thereto. In certain embodiments, the AAVrh98 VP1 protein has the amino acid sequence of SEQ ID NO: 14 or a sequence at least 95% identical thereto.

In certain embodiments, an rAAV is provided which has an rAAV97 capsid comprising a vector genome as described herein. An AAVrh97 capsid may be produced by expressing a nucleic acid sequence encoding the AAVrh97 VP1 of SEQ ID NO: 16. In other embodiments, an AAVrh97 capsid is produced by expressing SEQ ID NO: 15 or a sequence at least 99% identical thereto encoding SEQ ID NO: 16. In still other embodiments, AAV rh97 VP1 and VP3 proteins are characterized by a heterogenous population of VP proteins having the sequence of SEQ ID: 16 which is highly deamidated. In certain embodiments, the proteins are 75% to 100%, or 90% to 100% deamidated in at least position N57, N263, N384, and/or N514 based on the residue position numbers of SEQ ID NO: 16. The capsids may optionally be deamidated in other positions and/or may have other post-translation modifications.

In certain embodiment, an rAAV is provided which has an rAAV98 capsid comprising a vector genome as described herein. The AAVrh98 capsid may be produced by expressing a nucleic acid sequence encoding the AAVrh98 VP1 of SEQ ID NO: 28. In other embodiments, the AAVrh98 capsid is produced by expressing SEQ ID NO: 17 or a sequence at least 99% identical thereto encoding SEQ ID NO: 18. In still other embodiments, AAV rh98 VP1 and VP3 proteins are characterized by a heterogenous population of VP proteins having the sequence of SEQ ID 18 which is highly deamidated. In certain embodiments, the proteins are 75% to 100%, or 90% to 100% deamidated in at least position N57, N263, N384, and/or N514 based on the residue position numbers of SEQ ID NO: 18. The capsid proteins may optionally be deamidated in other positions and/or may have other post-translation modifications.

In certain embodiments, an rAAV is provided which has an AAVrh99 capsid having packaged therein a vector genome. The AAVrh99 capsid may be produced by expressing a nucleic acid sequence encoding the AAVrh99 VP1 of SEQ ID NO: 20. In certain embodiments, the AAVrh99 capsid is produced by expressing SEQ ID NO: 19 or a sequence at least 99% identical thereto encoding SEQ ID NO: 20. In still other embodiments, AAV rh99 VP1 and VP3 proteins are characterized by a heterogenous population of VP proteins having the sequence of SEQ ID NO: 20 which is highly deamidated. In certain embodiments, the proteins are 75% to 100%, or 90% to 100% deamidated in at least position N57, N263, N384, and/or N515 based on the residue positions of SEQ ID NO: 20. The capsid proteins may optionally be deamidated in other positions and/or may have other post-translation modifications.

In certain embodiments, provided herein are rAAV having a capsid of AAVrh96, AAVrh97, AAVrh98 or AAVrh99 comprising at least one of the vp1, vp2 and the vp3 of any of SEQ ID NO: 12, 14, 16, 18, 20, 27, 28, or 29, respectively. In certain embodiments, rAAV having a capsid protein comprising a vp1, vp2, and/or vp3 sequence at least 99% identical to AAVrh96, AAVrh97, AAVrh98 or AAVrh99 are provided. In certain embodiments, the vp1, vp2, and/or vp3 has up to 1, up to 2, up to 3, up to 4, up to 5, up to 6, up to 7, up to 8, up to 9, or up to 10 amino acid differences relative to the vp1, vp2, and/or vp3 of AAVrh96, AAVrh97, AAVrh98 or AAVrh99. Also provided herein are rAAV comprising AAV capsids encoded by at least one of the vp1, vp2, and vp3 of SEQ ID NO: 13, 15, 17 or 19, respectively or a sequence at least 99% identical to a SEQ ID Nos: 15 (AAVrh97), 17, (AAVrh98), or 19 (SEQ ID NO: 99). In certain embodiments, the sequence encodes a full-length vp1, vp2 and/or vp3 of AAVrh97, AAVrh98 or AAV99. In other embodiments, the vp1, vp2 and/or vp3 has an N-terminal and/or a C-terminal truncation (e.g. truncation(s) of about 1 to about 10 amino acids).

A “recombinant AAV” or “rAAV” is a DNAse-resistant viral particle containing two elements, an AAV capsid and a vector genome containing at least a non-AAV coding sequence packaged within the AAV capsid. Unless otherwise specified, this term may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

As used herein, a “vector genome” refers to the nucleic acid sequence packaged inside the rAAV capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other 1TRs nay be used. Further, the vector genone contains regulatory sequences which direct expression of the gene products. Suitable components of a vector genome are discussed in more detail herein. The vector genome is sometimes referred to herein as the “minigene”.

A rAAV is composed of an AAV capsid and a vector genome. An AAV capsid is an assembly of a heterogeneous population of vp1, a heterogeneous population of vp2, and a heterogeneous population of vp3 proteins. As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences.

As used herein, the term “heterogeneous population” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins may be at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs.

Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, at least 97%, at least 99%, or up to about 100% deamidated at a referenced amino acid position, as compared to the predicted amino acid sequence at the reference amino acid position. Such percentages may be determined using 2D-gel, mass spectrometry techniques, or other suitable techniques.

Without wishing to be bound by theory, the deamidation of at least highly deamidated residues in the vp proteins in the AAV capsid is believed to be primarily non-enzymatic in nature, being caused by functional groups within the capsid protein which deamidate selected asparagines, and to a lesser extent, glutamine residues. Efficient capsid assembly of the majority of deamidation vp1 proteins indicates that either these events occur following capsid assembly or that deamidation in individual monomers (vp1, vp2 or vp3) is well-tolerated structurally and largely does not affect assembly dynamics. Extensive deamidation in the VP1-unique (VP1-u) region (˜aa 1-137), generally considered to be located internally prior to cellular entry, suggests that VP deamidation may occur prior to capsid assembly.

Without wishing to be bound by theory, the deamidation of N may occur through its C-terminus residue's backbone nitrogen atom conducts a nucleophilic attack to the Asn side chain amide group carbon atom. An intermediate ring-closed succinimide residue is believed to form. The succinimide residue then conducts fast hydrolysis to lead to the final product aspartic acid (Asp) or iso aspartic acid (IsoAsp). Therefore, in certain embodiments, the deamidation of asparagine (N or Asn) leads to an Asp or IsoAsp, which may interconvert through the succinimide intermediate. As provided herein, each deamidated N in the VP1, VP2 or VP3 may independently be aspartic acid (Asp), isoaspartic acid (isoAsp), aspartate, and/or an interconverting blend of Asp and isoAsp, or combinations thereof. Any suitable ratio of α- and isoaspartic acid may be present. For example, in certain embodiments, the ratio may be from 10:1 to 1:10 aspartic to isoaspartic, about 50:50 aspartic: isoaspartic, or about 1:3 aspartic: isoaspartic, or another selected ratio. In certain embodiments, one or more glutamine (Q) may deamidates to glutamic acid (Glu), i.e., α-glutamic acid, γ-glutamic acid (Glu), or a blend of α- and γ-glutamic acid, which may interconvert through a common glutarinimide intermediate. Any suitable ratio of α- and γ-glutamic acid may be present. For example, in certain embodiments, the ratio may be from 10:1 to 1:10 α to γ, about 50:50 α:γ, or about 1:3 α:γ, or another selected ratio. Thus, an rAAV includes subpopulations within the rAAV capsid of vp1, vp2 and/or vp3 proteins with deamidated amino acids, including at a minimum, at least one subpopulation comprising at least one highly deamidated asparagine. In addition, other modifications may include isomerization, particularly at selected aspartic acid (D or Asp) residue positions. In still other embodiments, modifications may include an amidation at an Asp position.

In certain embodiments, an AAV capsid contains subpopulations of vp1, vp2 and vp3 having at least 1, at least 2, at least 3, at least 4, at least 5 to at least about 25 deamidated amino acid residue positions, of which at least 1 to 10%, at least 10 to 25%, at least 25 to 50%, at least 50 to 70%, at least 70 to 100%, at least 75 to 100%, at least 80-100%, or at least 90-100% are deamidated as compared to the encoded amino acid sequence of the vp proteins. The majority of these may be N residues. However, Q residues may also be deamidated.

As used herein, “encoded amino acid sequence” refers to the amino acid which is predicted based on the translation of a known DNA codon of a referenced nucleic acid sequence being translated to an amino acid. The following table illustrates DNA codons and twenty common amino acids, showing both the single letter code (SLC) and three letter code (3LC).

| Amino Acid | SLC | 3 LC | DNA codons |
|---|---|---|---|
| Isoleucine | I | Ile | ATT, ATC, ATA |
| Leucine | L | Leu | CTT, CTC, CTA, CTG, TTA, TTG |
| Valine | V | Val | GTT, GTC, GTA, GTG |
| Phenylalanine | F | Phe | TTT, TTC |
| Methionine | M | Met | ATG |
| Cysteine | C | Cys | TGT, TGC |
| Alanine | A | Ala | GCT, GCC, GCA, GCG |
| Glycine | G | Gly | GGT, GGC, GGA, GGG |
| Proline | P | Pro | CCT, CCC, CCA, CCG |
| Threonine | T | Thr | ACT, ACC, ACA, ACG |
| Serine | S | Ser | TCT, TCC, TCA, TCG, AGT, AGC |
| Tyrosine | Y | Tyr | TAT, TAC |
| Tryptophan | W | Trp | TGG |
| Glutamine | Q | Gln | CAA, CAG |
| Asparagine | N | Asn | AAT, AAC |
| Histidine | H | His | CAT, CAC |
| Glutamic acid | E | Glu | GAA, GAG |
| Aspartic acid | D | Asp | GAT, GAC |
| Lysine | K | Lys | AAA, AAG |
| Arginine | R | Arg | CGT, CGC, CGA, CGG, AGA, AGG |
| Stop codons | Stop | | TAA, TAG, TGA |

In certain embodiments, a rAAV has an AAV capsid having vp1, vp2 and vp3 proteins having subpopulations comprising combinations of two, three, four, five or more deamidated residues at the positions set forth in the tables provided herein and incorporated herein by reference.

Deamidation in the rAAV may be determined using 2D gel electrophoresis, and/or mass spectrometry, and/or protein modelling techniques. Online chromatography may be performed with an Acclaim PepMap column and a Thermo UltiMate 3000 RSLC system (Thermo Fisher Scientific) coupled to a Q Exactive HF with a NanoFlex source (Thermo Fisher Scientific). MS data is acquired using a data-dependent top-20 method for the Q Exactive HF, dynamically choosing the most abundant not-yet-sequenced precursor ions from the survey scans (200-2000 m/z). Sequencing is performed via higher energy collisional dissociation fragmentation with a target value of 1e5 ions determined with predictive automatic gain control and an isolation of precursors was performed with a window of 4 m/z. Survey scans were acquired at a resolution of 120,000 at m/z 200. Resolution for HCD spectra may be set to 30,000 at m/z200 with a maximum ion injection time of 50 ms and a normalized collision energy of 30. The S-lens RF level may be set at 50, to give optimal transmission of the m/z region occupied by the peptides from the digest. Precursor ions may be excluded with single, unassigned, or six and higher charge states from fragmentation selection. BioPharma Finder 1.0 software (Thermo Fischer Scientific) may be used for analysis of the data acquired. For peptide mapping, searches are performed using a single-entry protein FASTA database with carbamidomethylation set as a fixed modification; and oxidation, deamidation, and phosphorylation set as variable modifications, a 10-ppm mass accuracy, a high protease specificity, and a confidence level of 0.8 for MS/MS spectra. Examples of suitable proteases may include, e.g., trypsin or chymotrypsin. Mass spectrometric identification of deamidated peptides is relatively straightforward, as deamidation adds to the mass of intact molecule +0.984 Da (the mass difference between —OH and —NH groups). The percent deamidation of a particular peptide is determined by mass area of the deamidated peptide divided by the sum of the area of the deamidated and native peptides. Considering the number of possible deamidation sites, isobaric species which are deamidated at different sites may co-migrate in a single peak. Consequently, fragment ions originating from peptides with multiple potential deamidation sites can be used to locate or differentiate multiple sites of deamidation. In these cases, the relative intensities within the observed isotope patterns can be used to specifically determine the relative abundance of the different deamidated peptide isomers. This method assumes that the fragmentation efficiency for all isomeric species is the same and independent on the site of deamidation. It will be understood by one of skill in the art that a number of variations on these illustrative methods can be used. For example, suitable mass spectrometers may include, e.g, a quadrupole time of flight mass spectrometer (QTOF), such as a Waters Xevo or Agilent 6530 or an orbitrap instrument, such as the Orbitrap Fusion or Orbitrap Velos (Thermo Fisher). Suitably liquid chromatography systems include, e.g., Acquity UPLC system from Waters or Agilent systems (1100 or 1200 series). Suitable data analysis software may include, e.g., MassLynx (Waters), Pinpoint and Pepfinder (Thermo Fischer Scientific), Mascot (Matrix Science), Peaks DB (Bioinformatics Solutions). Still other techniques may be described, e.g., in X. Jin et al, Hu Gene Therapy Methods, Vol. 28, No. 5, pp. 255-267, published online Jun. 16, 2017.

In addition to deamidations, other modifications may occur do not result in conversion of one amino acid to a different amino acid residue. Such modifications may include acetylated residues, isomerizations, phosphorylations, or oxidations. In certain embodiments, the AAV is modified to change the glycine in an asparagine-glycine pair, to reduce deamidation, particularly a residue position which is not typically highly deamidated.

Amino acid modifications may be made by conventional genetic engineering techniques. For example, a nucleic acid sequence containing modified AAV vp codons may be generated in which one to three of the codons encoding glycine in asparagine-glycine pairs are modified to encode an amino acid other than glycine. In certain embodiments, a nucleic acid sequence containing modified asparagine codons may be engineered at one to three of the asparagine-glycine pairs, such that the modified codon encodes an amino acid other than asparagine. Each modified codon may encode a different amino acid. Alternatively, one or more of the altered codons may encode the same amino acid. Such mutant rAAV may have reduced immunogenicity and/or increase stability on storage, particularly storage in suspension form.

Also provided herein are nucleic acid sequences encoding the AAV capsids having reduced deamidation. It is within the skill in the art to design nucleic acid sequences encoding this AAV capsid, including DNA (genomic or cDNA), or RNA (e.g., mRNA). Such nucleic acid sequences may be codon-optimized for expression in a selected system (i.e., cell type) and can be designed by various methods. This optimization may be performed using methods which are available on-line (e.g., GeneArt), published methods, or a company which provides codon optimizing services, e.g., DNA2.0 (Menlo Park, CA). One codon optimizing method is described, e.g., in International Patent Publication No. WO 2015/012924, which is incorporated by reference herein in its entirety. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence and produce a nucleic acid fragment of a codon-optimized coding region which encodes the polypeptide. A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPOR vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

In certain embodiments, AAV capsids are provided which have a heterogeneous population of AAV capsid isoforms (i.e., VP1, VP2, VP3) which contain multiple highly deamidated “NG” positions. In certain embodiments, the highly deamidated positions are in the locations identified below, with reference to the predicted full-length VP1 amino acid sequence. In other embodiments, the capsid gene is modified such that the referenced “NG” is ablated and a mutant “NG” is engineered into another position.

B. rAAV Vectors and Compositions

In one aspect, provided herein are molecules which utilize the AAV capsid sequences described herein, including fragments thereof, for production of viral vectors useful in delivery of a heterologous gene or other nucleic acid sequences to a target cell. In certain embodiments, the rAAV provided have a capsid as described herein, and have packaged in the capsid a vector genome comprising a non-AAV nucleic acid sequence. In certain embodiments, the vectors useful in compositions and methods described herein contain, at a minimum, sequences encoding a selected AAV capsid as described herein, e.g., an AAVrh94, AAVrh95, AAVrh96, AAVrh97, AAVrh98, or AAVrh99 capsid, or a fragment thereof, including the vp1, vp2, or vp3 capsid protein. In certain embodiments, useful vectors contain, at a minimum, sequences encoding a selected AAV serotype rep protein, or a fragment thereof. Optionally, such vectors may contain both AAV cap and rep proteins. In vectors in which both AAV rep and cap are provided, the AAV rep and AAV cap sequences can both be of one serotype origin. Alternatively, vectors may be used in which the rep sequences are from an AAV which differs from the wild type AAV providing the cap sequences, e.g., the same AAV providing the ITRs and rep.

In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector, such as AAV2/8 described in U.S. Pat. No. 7,282,199, which is incorporated by reference herein. Optionally, the vectors further contain a minigene comprising a selected transgene which is flanked by AAV 5′ ITR and AAV 3′ ITR. In another embodiment, the AAV is a self-complementary AAV (sc-AAV) (See, US 2012/0141422 which is incorporated herein by reference). Self-complementary vectors package an inverted repeat genome that can fold into dsDNA without the requirement for DNA synthesis or base-pairing between multiple vector genomes. Because scAAV have no need to convert the single-stranded DNA (ssDNA) genome into double-stranded DNA (dsDNA) prior to expression, they are more efficient vectors. However, the trade-off for this efficiency is the loss of half the coding capacity of the vector, ScAAV are useful for small protein-coding genes (up to ˜55 kd) and any currently available RNA-based therapy.

Pseudotyped vectors, wherein the capsid of one AAV is replaced with a heterologous capsid protein, are useful herein. For example, AAV vectors utilizing an AAVrh94, AAVrh95, AAVrh96, AAVrh97, AAVrh98, or AAVrh99 capsid as described herein, have AAV2 ITRs. See, Mussolini et al. Unless otherwise specified, the AAV ITRs, and other selected AAV components described herein, may be individually selected from among any AAV serotype, including, without limitation, AAV1, AAV2, AAV3, AAV4, AAV5, AAV6, AAV7, AAV8, AAV9, rh37, hu68, or other known and unknown AAV serotypes. In one desirable embodiment, the ITRs of AAV serotype 2 are used. However, ITRs from other suitable serotypes may be selected. These ITRs or other AAV components may be readily isolated using techniques available to those of skill in the art from an AAV serotype. Such AAV may be isolated or obtained from academic, commercial, or public sources (e.g., the American Type Culture Collection, Manassas, VA). Alternatively, the AAV sequences may be obtained through synthetic or other suitable means by reference to published sequences such as are available in the literature or in databases such as, e.g., GenBank, PubMed, or the like.

The rAAV provided herein comprise a vector genome. The vector genome is composed of, at a minimum, a non-AAV or heterologous nucleic acid sequence (e.g., a transgene), as described below, regulatory sequences, and 5′ and 3′ AAV inverted terminal repeats (ITRs). It is this minigene which is packaged into a capsid protein and delivered to a selected target cell or target tissue.

The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a target cell. The heterologous nucleic acid sequence (transgene) can be derived from any organism. The AAV may comprise one or more transgenes.

As used herein, the terms “target cell” and “target tissue” can refer to any cell or tissue which is intended to be transduced by the subject AAV vector. The term may refer to any one or more of muscle, liver, lung, airway epithelium, central nervous system, neurons, eye (ocular cells), or heart. In one embodiment, the target tissue is liver. In another embodiment, the target tissue is the heart. In another embodiment, the target tissue is brain. In another embodiment, the target tissue is muscle.

As used herein, the term “mammalian subject” or “subject” includes any mammal in need of the methods of treatment described herein or prophylaxis, including particularly humans. Other mammals in need of such treatment or prophylaxis include dogs, cats, or other domesticated animals, horses, livestock, laboratory animals, including non-human primates, etc. The subject may be male or female.

As used herein, the term “host cell” may refer to the packaging cell line in which the rAAV is produced from the plasmid. In the alternative, the term “host cell” may refer to a target cell in which expression of the transgene is desired.

### Therapeutic Transgenes

Useful products encoded by the transgene include a variety of gene products which replace a defective or deficient gene, inactivate or “knock-out”, or “knock-down” or reduce the expression of a gene which is expressing at an undesirably high level, or delivering a gene product which has a desired therapeutic effect. In most embodiments, the therapy will be “somatic gene therapy”, i.e., transfer of genes to a cell of the body which does not produce sperm or eggs. In certain embodiments, the transgenes express proteins have the sequence of native human sequences. However, in other embodiments, synthetic proteins are expressed. Such proteins may be intended for treatment of humans, or in other embodiments, designed for treatment of animals, including companion animals such as canine or feline populations, or for treatment of livestock or other animals which come into contact with human populations.

Examples of suitable gene products may include those associated with familial hypercholesterolemia, muscular dystrophy, cystic fibrosis, and rare or orphan diseases. Examples of such rare disease may include spinal muscular atrophy (SMA), Huntingdon's Disease, Rett Syndrome (e.g., methyl-CpG-binding protein 2 (MeCP2); UniProtKB-P51608), Amyotrophic Lateral Sclerosis (ALS), Duchenne Type Muscular dystrophy, Friedrichs Ataxia (e.g., frataxin), ATXN2 associated with spinocerebellar ataxia type 2 (SCA2)/ALS; TDP-43 associated with ALS, progranulin (PRGN) (associated with non-Alzheimer's cerebral degenerations, including, frontotemporal dementia (FTD), progressive non-fluent aphasia (PNFA) and semantic dementia), among others. See, e.g., orpha_net/consor/cgi-bin/Disease_Search_List.php; rarediseases_info_nih_gov/diseases. In one embodiment, the transgene is not human low-density lipoprotein receptor (hLDLR). In another embodiment, the transgene is not an engineered human low-density lipoprotein receptor (hLDLR) variant, such as those described in WO 2015/164778.

Examples of suitable genes may include, e.g., hormones and growth and differentiation factors including, without limitation, insulin, glucagon, glucagon-like peptide-1 (GLP1), growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO) (including, e.g., human, canine or feline epo), connective tissue growth factor (CTGF), neutrophic factors including, e.g., basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor α superfamily, including TGFα, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgene products include proteins that regulate the immune system including, without limitation, cytokines and lymphokines such as thrombopoietin (TPO), interleukins (IL) IL-1 through IL-36 (including, e.g., human interleukins IL-1, IL-1α, IL-1β, IL-2, IL-3, IL-4, IL-6, IL-8, IL-12, IL-11, IL-12, IL-13, IL-18, IL-31, IL-35), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. For example, in certain embodiments, the rAAV antibodies may be designed to delivery canine or feline antibodies, e.g., such as anti-IgE, anti-IL31, anti-IL33, anti-CD20, anti-NGF, anti-GnRH Useful gene products also include complement regulatory proteins such as complement regulatory proteins, membrane cofactor protein (MCP), decay accelerating factor (DAF), CR1, CF2, CD59, and Cl esterase inhibitor (CJ-INH).

Still other useful gene products include any one of the receptors for the hormones, growth factors, cytokines, lymphokines, regulatory proteins and immune system proteins. The invention encompasses receptors for cholesterol regulation and/or lipid modulation, including the low-density lipoprotein (LDL) receptor, high density lipoprotein (HDL) receptor, the very low density lipoprotein (VLDL) receptor, and scavenger receptors. The invention also encompasses gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenm, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include hydroxymethylbilane synthase (HMBS), carbamoyl synthetase I, ornithine transcarbamylase (OTC), arginosuccinate synthetase, arginosuccinate lyase (ASL) for treatment of arginosuccinate lyase deficiency, arginase, fumarylacetate hydrolase, phenylalanine hydroxylase, alpha-1 antitrypsin, rhesus alpha-fetoprotein (AFP), chorionic gonadotrophin (CG), glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding β-glucuronidase (GUSB)). In another example, the gene product is ubiquitin protein ligase E3A (UBE3A). Still useful gene products include UDP Glucuronosyltransferase Family 1 Member A1 (UGT1A1).

In certain embodiments, the rAAV may be used in gene editing systems, which system may involve one rAAV or co-administration of multiple rAAV stocks. For example, the rAAV may be engineered to deliver SpCas9, SaCas9, ARCUS, Cpf1 (also known as Cas12a), CjCas9, and other suitable gene editing constructs.

Still other useful gene products include those used for treatment of hemophilia, including hemophilia B (including Factor IX) and hemophilia A (including Factor VIII and its variants, such as the light chain and heavy chain of the heterodimer and the B-deleted domain; U.S. Pat. Nos. 6,200,560 and 6,221,349). In some embodiments, the minigene comprises first 57 base pairs of the Factor VIII heavy chain which encodes the 10 amino acid signal sequence, as well as the human growth hormone (hGH) polyadenylation sequence. In alternative embodiments, the minigene further comprises the A1 and A2 domains, as well as 5 amino acids from the N-terminus of the B domain, and/or 85 amino acids of the C-terminus of the B domain, as well as the A3, C1 and C2 domains. In yet other embodiments, the nucleic acids encoding Factor VIII heavy chain and light chain are provided in a single minigene separated by 42 nucleic acids coding for 14 amino acids of the B domain [U.S. Pat. No. 6,200,560].

Other useful gene products include non-naturally occurring polypeptides, such as chimeric or hybrid polypeptides having a non-naturally occurring amino acid sequence containing insertions, deletions, or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target.

Reduction and/or modulation of expression of a gene is particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable therapeutic polypeptides and proteins include those which may be useful for treating individuals suffering from autoimmune diseases and disorders by conferring a broad based protective immune response against targets that are associated with autoimmunity including cell receptors and cells which produce “self”-directed antibodies. T cell mediated autoimmune diseases include Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

Further illustrative genes which may be delivered via the rAAV provided herein for treatment of, for example, liver indications include, without limitation, glucose-6-phosphatase, associated with glycogen storage disease or deficiency type 1A (GSD1), phosphoenolpyruvate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase (PAH), associated with phenylketonuria (PKU); gene products associated with Primary Hyperoxaluria Type 1 including Hydroxyacid Oxidase 1 (GO/HAO1) and AGXT, branched chain alpha-ketoacid dehydrogenase, including BCKDH, BCKDH-E2, BAKDH-E1a, and BAKDH-E1b, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; orithine transcarbamylase (OTC), associated with ornithine transcarbamylase deficiency; argininosuccinic acid synthetase (ASS1), associated with citrullinemia; lecithin-cholesterol acyltransferase (LCAT) deficiency; amethylmalonic acidemia (MMA); NPC1 associated with Niemann-Pick disease, type Cl); propionic academia (PA); TTR associated with Transthyretin (TTR)-related Hereditary Amyloidosis; low density lipoprotein receptor (LDLR) protein, associated with familial hypercholesterolemia (FH), LDLR variant, such as those described in WO 2015/164778; PCSK9; ApoE and ApoC proteins, associated with dementia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); beta-galactosidase (GLB1) associated with GM1 gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosuccinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; α-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

Additional genes and diseases of interest include, e.g., dystonin gene related diseases such as Hereditary Sensory and Autonomic Neuropathy Type VI (the DST gene encodes dystonin; dual AAV vectors may be required due to the size of the protein (˜7570 aa); SCN9A related diseases, in which loss of function mutants cause inability to feel pain and gain of function mutants cause pain conditions, such as erythromelagia. Another condition is Charcot-Marie-Tooth (CMT) type 1F and 2E due to mutations in the NEFL gene (neurofilament light chain) characterized by a progressive peripheral motor and sensory neuropathy with variable clinical and electrophysiologic expression. Other gene products associated with CMT include mitofusin 2 (MFN2).

In certain embodiments, the rAAV described herein may be used in treatment of mucopolysaccaridoses (MPS) disorders. Such rAAV may contain carry a nucleic acid sequence encoding α-L-iduronidase (IDUA) for treating MPS I (Hurler, Hurler-Scheie and Scheie syndromes); a nucleic acid sequence encoding iduronate-2-sulfatase (IDS) for treating MPS II (Hunter syndrome); a nucleic acid sequence encoding sulfamidase (SGSH) for treating MPSIII A, B, C, and D (Sanfilippo syndrome); a nucleic acid sequence encoding N-acetylgalactosamine-6-sulfate sulfatase (GALNS) for treating MPS IV A and B (Morquio syndrome); a nucleic acid sequence encoding arylsulfatase B (ARSB) for treating MPS VI (Maroteaux-Lamy syndrome); a nucleic acid sequence encoding hyaluronidase for treating MPSI IX (hyaluronidase deficiency) and a nucleic acid sequence encoding beta-glucuronidase for treating MPS VII (Sly syndrome).

In some embodiments, an rAAV vector comprising a nucleic acid encoding a gene product associated with cancer (e.g., tumor suppressors) may be used to treat the cancer, by administering a rAAV harboring the rAAV vector to a subject having the cancer. In some embodiments, an rAAV vector comprising a nucleic acid encoding a small interfering nucleic acid (e.g., shRNAs, miRNAs) that inhibits the expression of a gene product associated with cancer (e.g., oncogenes) may be used to treat the cancer, by administering a rAAV harboring the rAAV vector to a subject having the cancer. In some embodiments, an rAAV vector comprising a nucleic acid encoding a gene product associated with cancer (or a functional RNA that inhibits the expression of a gene associated with cancer) may be used for research purposes, e.g., to study the cancer or to identify therapeutics that treat the cancer. The following is a non-limiting list of exemplary genes known to be associated with the development of cancer (e.g., oncogenes and tumor suppressors): AARS, ABCB1, ABCC4, ABI2, ABL1, ABL2, ACK1, ACP2, ACY1, ADSL, AK1, AKR1C2, AKT1, ALB, ANPEP, ANXA5, ANXA7, AP2M1, APC, ARHGAP5, ARHGEF5, ARID4A, ASNS, ATF4, ATM, ATP5B, ATP50, AXL, BARD1, BAX, BCL2, BHLHB2, BLMH, BRAF, BRCA1, BRCA2, BTK, CANX, CAP1, CAPN1, CAPNS1, CAV1, CBFB, CBLB, CCL2, CCND1, CCND2, CCND3, CCNE1, CCT5, CCYR61, CD24, CD44, CD59, CDC20, CDC25, CDC25A, CDC25B, CDC2L5, CDK10, CDK4, CDK5, CDK9, CDKL1, CDKN1A, CDKN1B, CDKN1C, CDKN2A, CDKN2B, CDKN2D, CEBPG, CENPC1, CGRRF1, CHAF1A, CIB1, CKMT1, CLK1, CLK2, CLK3, CLNS1A, CLTC, COL1A1, COL6A3, COX6C, COX7A2, CRAT, CRHR1, CSF1R, CSK, CSNK1G2, CTNNA1, CTNNB1, CTPS, CTSC, CTSD, CUL1, CYR61, DCC, DCN, DDX10, DEK, DHCR7, DHRS2, DHX8, DLG3, DVL1, DVL3, E2F1, E2F3, E2F5, EGFR, EGR1, EIF5, EPHA2, ERBB2, ERBB3, ERBB4, ERCC3, ETV1, ETV3, ETV6, F2R, FASTK, FBN1, FBN2, FES, FGFR1, FGR, FKBP8, FN1, FOS, FOSL1, FOSL2, FOXG1A, FOXO1A, FRAP1, FRZB, FTL, FZD2, FZD5, FZD9, G22P1, GAS6, GCN5L2, GDF15, GNA13, GNAS, GNB2, GNB2L1, GPR39, GRB2, GSK3A, GSPT1, GTF2I, HDAC1, HDGF, HMMR, HPRT1, HRB, HSPA4, HSPA5, HSPA8, HSPB1, HSPH1, HYAL1, HYOU1, ICAM1, ID1, ID2, IDUA, IER3, IFITM1, IGF1R, IGF2R, IGFBP3, IGFBP4, IGFBP5, IL1B, ILK, ING1, IRF3, ITGA3, ITGA6, ITGB4, JAK1, JARIDIA, JUN, JUNB, JUND, K-ALPHA-1, KIT, KITLG, KLK10, KPNA2, KRAS2, KRT18, KRT2A, KRT9, LAMB1, LAMP2, LCK, LCN2, LEP, LITAF, LRPAP1, LTF, LYN, LZTR1, MADH1, MAP2K2, MAP3K8, MAPK12, MAPK13, MAPKAPK3, MAPRE1, MARS, MAS1, MCC, MCM2, MCM4, MDM2, MDM4, MET, MGST1, MICB, MLLT3, MME, MMP1, MMP14, MMP17, MMP2, MNDA, MSH2, MSH6, MT3, MYB, MYBL1, MYBL2, MYC, MYCL1, MYCN, MYD88, MYL9, MYLK, NEO1, NF1, NF2, NFKB1, NFKB2, NFSF7, NID, NINE, NMBR, NME1, NME2, NME3, NOTCH1, NOTCH2, NOTCH4, NPM1, NQOl, NR1D1, NR2F1, NR2F6, NRAS, NRG1, NSEP1, OSM, PA2G4, PABPC1, PCNA, PCTK1, PCTK2, PCTK3, PDGFA, PDGFB, PDGFRA, PDPK1, PEA15, PFDN4, PFDN5, PGAM1, PHB, PIK3CA, PIK3CB, PIK3CG, PIM1, PKM2, PKMYT1, PLK2, PPARD, PPARG, PPIH, PPPICA, PPP2R5A, PRDX2, PRDX4, PRKARIA, PRKCBPI, PRNP, PRSS15, PSMA1, PTCH, PTEN, PTGS1, PTMA, PTN, PTPRN, RAB5A, RAC1, RAD50, RAF1, RALBP1, RAPlA, RARA, RARB, RASGRF1, RB1, RBBP4, RBL2, REA, REL, RELA, RELB, RET, RFC2, RGS19, RHOA, RHOB, RHOC, RHOD, RIPK1, RPN2, RPS6 KB1, RRM1, SARS, SELENBP1, SEMA3C, SEMA4D, SEPP1, SERPINH1, SFN, SFPQ, SFRS7, SHB, SHH, SIAH2, SIVA, SIVA TP53, SKI, SKIL, SLC16A1, SLC1A4, SLC20A1, SMO, sphingomyelinphosphodiesterase 1 (SMPD1), SNAI2, SND1, SNRPB2, SOCSl, SOCS3, SOD1, SORT1, SPINT2, SPRY2, SRC, SRPX, STAT1, STAT2, STAT3, STAT5B, STC1, TAF1, TBL3, TBRG4, TCF1, TCF7L2, TFAP2C, TFDP1, TFDP2, TGFA, TGFB1, TGFB1, TGFBR2, TGFBR3, THBS1, TIE, TIMP1, TIMP3, TJP1, TK1, TLE1, TNF, TNFRSF10A, TNFRSF10B, TNFRSF1A, TNFRSF1B, TNFRSF6, TNFSF7, TNK1, TOB1, TP53, TP53BP2, TP5313, TP73, TPBG, TPT1, TRADD, TRAM1, TRRAP, TSG101, TUFM, TXNRD1, TYRO3, UBC, UBE2L6, UCHL 1, USP7, VDAC1, VEGF, VHL, VIL2, WEE1, WNT1, WNT2, WNT2B, WNT3, WNT5A, WT1, XRCC1, YES1, YWHAB, YWHAZ, ZAP70, and ZNF9.

A rAAV vector may comprise as a transgene, a nucleic acid encoding a protein or functional RNA that modulates apoptosis. The following is a non-limiting list of genes associated with apoptosis and nucleic acids encoding the products of these genes and their homologues and encoding small interfering nucleic acids (e.g., shRNAs, miRNAs) that inhibit the expression of these genes and their homologues are useful as transgenes in certain embodiments of the invention: RPS27A, ABL1, AKT1, APAF1, BAD, BAG1, BAG3, BAG4, BAK1, BAX, BCL10, BCL2, BCL2A1, BCL2L1, BCL2L10, BCL2L11, BCL2L12, BCL2L13, BCL2L2, BCLAF1, BFAR, BID, BIK, NAIP, BIRC2, BIRC3, XIAP, BIRC5, BIRC6, BIRC7, BIRC8, BNIP1, BNIP2, BNIP3, BNIP3L, BOK, BRAF, CARD10, CARDl1, NLRC4, CARD14, NOD2, NOD1, CARD6, CARDS, CARDS, CASP1, CASP10, CASP14, CASP2, CASP3, CASP4, CASP5, CASP6, CASP7, CASP8, CASP9, CFLAR, CIDEA, CIDEB, CRADD, DAPK1, DAPK2, DFFA, DFFB, FADD, GADD45A. GDNF, HRK, IGFIR, LTA, LTBR, MCL1, NOL3, PYCARD, RIPK1, RIPK2, TNF, TNFRSF10A, TNFRSF10B, TNFRSF10C, TNFRSF10D, TNFRSF11B, TNFRSF12A, TNFRSF14, TNFRSF19, TNFRSF1A, TNFRSF1B, TNFRSF21, TNFRSF25, CD40, FAS, TNFRSF6B, CD27, TNFRSF9, TNFSF10, TNFSF14, TNFSF18, CD40LG, FASLG, CD70, TNFSF8, TNFSF9, TP53, TP53BP2, TP73, TP63, TRADD, TRAF1, TRAF2, TRAF3, TRAF4, and TRAF5.

Useful transgene products also include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

The following non-limiting list of miRNA genes, and their homologues, are useful as transgenes or as targets for small interfering nucleic acids encoded by transgenes (e.g., miRNA sponges, antisense oligonucleotides, TuD RNAs) in certain embodiments of the methods: hsa-let-7a, hsa-let-7a*, hsa-let-7b, hsa-let-7b*, hsa-let-7c, hsa-let-7c*, hsa-let-7d, hsa-let-7d*, hsa-let-7e, hsa-let-7e*, hsa-let-7f, hsa-let-7f-1*, hsa-let-7f-2*, hsa-let-7g, hsa-let-7g*, hsa-let-71, hsa-let-71*, hsa-miR-1, hsa-miR-100, hsa-miR-100*, hsa-miR-101, hsa-miR-101*, hsa-miR-103, hsa-miR-105, hsa-miR-105*, hsa-miR-106a, hsa-miR-106a*, hsa-miR-106b, hsa-miR-106b*, hsa-miR-107, hsa-miR-10a, hsa-miR-10a*, hsa-miR-10b, hsa-miR-10b*, hsa-miR-1178, hsa-miR-1179, hsa-miR-1180, hsa-miR-1181, hsa-miR-1182, hsa-miR-1183, hsa-miR-1184, hsa-miR-1185, hsa-miR-1197, hsa-miR-1200, hsa-miR-1201, hsa-miR-1202, hsa-miR-1203, hsa-miR-1204, hsa-miR-1205, hsa-miR-1206, hsa-miR-1207-3p, hsa-miR-1207-5p, hsa-miR-1208, hsa-miR-122, hsa-miR-122*, hsa-miR-1224-3p, hsa-miR-1224-5p, hsa-miR-1225-3p, hsa-miR-1225-5p, hsa-miR-1226, hsa-miR-1226*, hsa-miR-1227, hsa-miR-1228, hsa-miR-1228*, hsa-miR-1229, hsa-miR-1231, hsa-miR-1233, hsa-miR-1234, hsa-miR-1236, hsa-miR-1237, hsa-miR-1238, hsa-miR-124, hsa-miR-124*, hsa-miR-1243, hsa-miR-1244, hsa-miR-1245, hsa-miR-1246, hsa-miR-1247, hsa-miR-1248, hsa-miR-1249, hsa-miR-1250, hsa-miR-1251, hsa-miR-1252, hsa-miR-1253, hsa-miR-1254, hsa-miR-1255a, hsa-miR-1255b, hsa-miR-1256, hsa-miR-1257, hsa-miR-1258, hsa-miR-1259, hsa-miR-125a-3p, hsa-miR-125a-5p, hsa-miR-125b, hsa-miR-125b-1*, hsa-miR-125b-2*, hsa-miR-126, hsa-miR-126*, hsa-miR-1260, hsa-miR-1261, hsa-miR-1262, hsa-miR-1263, hsa-miR-1264, hsa-miR-1265, hsa-miR-1266, hsa-miR-1267, hsa-miR-1268, hsa-miR-1269, hsa-miR-1270, hsa-miR-1271, hsa-miR-1272, hsa-miR-1273, hsa-miR-127-3p, hsa-miR-1274a, hsa-miR-1274b, hsa-miR-1275, hsa-miR-127-5p, hsa-miR-1276, hsa-miR-1277, hsa-miR-1278, hsa-miR-1279, hsa-miR-128, hsa-miR-1280, hsa-miR-1281, hsa-miR-1282, hsa-miR-1283, hsa-miR-1284, hsa-miR-1285, hsa-miR-1286, hsa-miR-1287, hsa-miR-1288, hsa-miR-1289, hsa-miR-129*, hsa-miR-1290, hsa-miR-1291, hsa-miR-1292, hsa-miR-1293, hsa-miR-129-3p, hsa-miR-1294, hsa-miR-1295, hsa-miR-129-5p, hsa-miR-1296, hsa-miR-1297, hsa-miR-1298, hsa-miR-1299, hsa-miR-1300, hsa-miR-1301, hsa-miR-1302, hsa-miR-1303, hsa-miR-1304, hsa-miR-1305, hsa-miR-1306, hsa-miR-1307, hsa-miR-1308, hsa-miR-130a, hsa-miR-130a*, hsa-miR-130b, hsa-miR-130b*, hsa-miR-132, hsa-miR-132*, hsa-miR-1321, hsa-miR-1322, hsa-miR-1323, hsa-miR-1324, hsa-miR-133a, hsa-miR-133b, hsa-miR-134, hsa-miR-135a, hsa-miR-135a*, hsa-miR-135b, hsa-miR-135b*, hsa-miR-136, hsa-miR-136*, hsa-miR-137, hsa-miR-138, hsa-miR-138-1*, hsa-miR-138-2*, hsa-miR-139-3p, hsa-miR-139-5p, hsa-miR-140-3p, hsa-miR-140-5p, hsa-miR-141, hsa-miR-141*, hsa-miR-142-3p, hsa-miR-142-5p, hsa-miR-143, hsa-miR-143*, hsa-miR-144, hsa-miR-144*, hsa-miR-145, hmiRsa-miR-145*, hsa-miR-146a, hsa-miR-146a*, hsa-miR-146b-3p, hsa-miR-146b-5p, hsa-miR-147, hsa-miR-147b, hsa-miR-148a, hsa-miR-148a*, hsa-miR-148b, hsa-miR-148b*, hsa-miR-149, hsa-miR-149*, hsa-miR-150, hsa-miR-150*, hsa-miR-151-3p, hsa-miR-151-5p, hsa-miR-152, hsa-miR-153, hsa-miR-154, hsa-miR-154*, hsa-miR-155, hsa-miR-155*, hsa-miR-15a, hsa-miR-15a*, hsa-miR-15b, hsa-miR-15b*, hsa-miR-16, hsa-miR-16-8*, hsa-miR-16-2*, hsa-miR-17, hsa-miR-17*, hsa-miR-1818a, hsa-miR-18a*, hsa-miR-181a-2*, hsa-miR-185b, hsa-miR-181c, hsa-miR-181*, hsa-miR-18d, hsa-miR-182, hsa-miR-182*, hsa-miR-1825, hsa-miR-1826, hsa-miR-1827, hsa-miR-183, hsa-miR-183*, hsa-miR-184, hsa-miR-185, hsa-miR-185*, hsa-miR-186, hsa-miR-186*, hsa-miR-187, hsa-miR-187*, hsa-miR-188-3p, hsa-miR-188-5p, hsa-miR-18a, hsa-miR-18a*, hsa-miR-18b, hsa-miR-18b*, hsa-miR-190, hsa-miR-190b, hsa-miR-191, hsa-miR-191*, hsa-miR-192, hsa-miR-192*, hsa-miR-193a-3p, hsa-miR-193a-5p, hsa-miR-193b, hsa-miR-193b*, hsa-miR-194, hsa-miR-194*, hsa-miR-195, hsa-miR-195*, hsa-miR-196a, hsa-miR-196a*, hsa-miR-196b, hsa-miR-197, hsa-miR-198, hsa-miR-199a-3p, hsa-miR-199a-5p, hsa-miR-199b-5p, hsa-miR-19a, hsa-miR-19a*, hsa-miR-19b, hsa-miR-19b-1*, hsa-miR-19b-2*, hsa-miR-200a, hsa-miR-200a*, hsa-miR-200b, hsa-miR-200b*, hsa-miR-200c, hsa-miR-200c*, hsa-miR-202, hsa-miR-202*, hsa-miR-203, hsa-miR-204, hsa-miR-205, hsa-miR-206, hsa-miR-208a, hsa-miR-208b, hsa-miR-20a, hsa-miR-20a*, hsa-miR-20b, hsa-miR-20b*, hsa-miR-21, hsa-miR-21*, hsa-miR-210, hsa-miR-211, hsa-miR-212, hsa-miR-214, hsa-miR-214*, hsa-miR-215, hsa-miR-216a, hsa-miR-216b, hsa-miR-217, hsa-miR-218, hsa-miR-218-1*, hsa-miR-218-2*, hsa-miR-219-1-3p, hsa-miR-219-2-3p, hsa-miR-219-5p, hsa-miR-22, hsa-miR-22*, hsa-miR-220a, hsa-miR-220b, hsa-miR-220c, hsa-miR-221, hsa-miR-221*, hsa-miR-222, hsa-miR-222*, hsa-miR-223, hsa-miR-223*, hsa-miR-224, hsa-miR-23a, hsa-miR-23a*, hsa-miR-23b, hsa-miR-23b*, hsa-miR-24, hsa-miR-24-1*, hsa-miR-24-2*, hsa-miR-25, hsa-miR-25*, hsa-miR-26a, hsa-miR-26a-1*, hsa-miR-26a-2*, hsa-miR-26b, hsa-miR-26b*, hsa-miR-27a, hsa-miR-27a*, hsa-miR-27b, hsa-miR-27b*, hsa-miR-28-3p, hsa-miR-28-5p, hsa-miR-296-3p, hsa-miR-296-5p, hsa-miR-297, hsa-miR-298, hsa-miR-299-3p, hsa-miR-299-5p, hsa-miR-29a, hsa-miR-29a*, hsa-miR-29b, hsa-miR-296-1*, hsa-miR-296-2*, hsa-miR-29c, hsa-miR-29c*, hsa-miR-300, hsa-miR-301a, hsa-miR-301b, hsa-miR-302a, hsa-miR-302a*, hsa-miR-302b, hsa-miR-302b*, hsa-miR-302c, hsa-miR-302c*, hsa-miR-302d, hsa-miR-302d*, hsa-miR-302e, hsa-miR-302f, hsa-miR-30a, hsa-miR-30a*, hsa-miR-30b, hsa-miR-30b*, hsa-miR-30c, hsa-miR-30c-1*, hsa-miR-30c-2*, hsa-miR-30d, hsa-miR-30d*, hsa-miR-30e, hsa-miR-30e*, hsa-miR-31, hsa-miR-31*, hsa-miR-32, hsa-miR-32*, hsa-miR-320a, hsa-miR-320b, hsa-miR-320c, hsa-miR-320d, hsa-miR-323-3p, hsa-miR-323-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335, hsa-miR-335*, hsa-miR-337-3p, hsa-miR-337-5p, hsa-miR-338-3p, hsa-miR-338-5p, hsa-miR-339-3p, hsa-miR-339-5p, hsa-miR-33a, hsa-miR-33a*, hsa-miR-33b, hsa-miR-33b*, hsa-miR-340, hsa-miR-340*, hsa-miR-342-3p, hsa-miR-342-5p, hsa-miR-345, hsa-miR-346, hsa-miR-34a, hsa-miR-34a*, hsa-miR-34b, hsa-miR-34b*, hsa-miR-34c-3p, hsa-miR-34c-5p, hsa-miR-361-3p, hsa-miR-361-5p, hsa-miR-362-3p, hsa-miR-362-5p, hsa-miR-363, hsa-miR-363*, hsa-miR-365, hsa-miR-367, hsa-miR-367*, hsa-miR-369-3p, hsa-miR-369-5p, hsa-miR-370, hsa-miR-371-3p, hsa-miR-371-5p, hsa-miR-372, hsa-miR-373, hsa-miR-373*, hsa-miR-374a, hsa-miR-374a*, hsa-miR-374b, hsa-miR-374b*, hsa-miR-375, hsa-miR-376a, hsa-miR-376a*, hsa-miR-376b, hsa-miR-376c, hsa-miR-377, hsa-miR-377*, hsa-miR-378, hsa-miR-378*, hsa-miR-379, hsa-miR-379*, hsa-miR-380, hsa-miR-380*, hsa-miR-381, hsa-miR-382, hsa-miR-383, hsa-miR-384, hsa-miR-409-3p, hsa-miR-409-5p, hsa-miR-410, hsa-miR-411, hsa-miR-411*, hsa-miR-412, hsa-miR-421, hsa-miR-422a, hsa-miR-423-3p, hsa-miR-423-5p, hsa-miR-424, hsa-miR-424*, hsa-miR-425, hsa-miR-425*, hsa-miR-429, hsa-miR-431, hsa-miR-431*, hsa-miR-432, hsa-miR-432*, hsa-miR-433, hsa-miR-448, hsa-miR-449a, hsa-miR-449b, hsa-miR-450a, hsa-miR-450b-3p, hsa-miR-450b-5p, hsa-miR-451, hsa-miR-452, hsa-miR-452*, hsa-miR-453, hsa-miR-454, hsa-miR-454*, hsa-miR-455-3p, hsa-miR-455-5p, hsa-miR-483-3p, hsa-miR-483-5p, hsa-miR-484, hsa-miR-485-3p, hsa-miR-485-5p, hsa-miR-486-3p, hsa-miR-486-5p, hsa-miR-487a, hsa-miR-487b, hsa-miR-488, hsa-miR-488*, hsa-miR-489, hsa-miR-490-3p, hsa-miR-490-5p, hsa-miR-491-3p, hsa-miR-491-5p, hsa-miR-492, hsa-miR-493, hsa-miR-493*, hsa-miR-494, hsa-miR-495, hsa-miR-496, hsa-miR-497, hsa-miR-497*, hsa-miR-498, hsa-miR-499-3p, hsa-miR-499-5p, hsa-miR-500, hsa-miR-500*, hsa-miR-501-3p, hsa-miR-501-5p, hsa-miR-502-3p, hsa-miR-502-5p, hsa-miR-503, hsa-miR-504, hsa-miR-505, hsa-miR-505*, hsa-miR-506, hsa-miR-507, hsa-miR-508-3p, hsa-miR-508-5p, hsa-miR-509-3-5p, hsa-miR-509-3p, hsa-miR-509-5p, hsa-miR-510, hsa-miR-511, hsa-miR-512-3p, hsa-miR-512-5p, hsa-miR-513a-3p, hsa-miR-513a-5p, hsa-miR-513b, hsa-miR-513c, hsa-miR-514, hsa-miR-515-3p, hsa-miR-515-5p, hsa-miR-516a-3p, hsa-miR-516a-5p, hsa-miR-516b, hsa-miR-517*, hsa-miR-517a, hsa-miR-517b, hsa-miR-517c, hsa-miR-518a-3p, hsa-miR-518a-5p, hsa-miR-518b, hsa-miR-518c, hsa-miR-518c*, hsa-miR-518d-3p, hsa-miR-518d-5p, hsa-miR-518e, hsa-miR-518e*, hsa-miR-518f, hsa-miR-518f*, hsa-miR-519a, hsa-miR-519b-3p, hsa-miR-519c-3p, hsa-miR-519d, hsa-miR-519e, hsa-miR-519e*, hsa-miR-520a-3p, hsa-miR-520a-5p, hsa-miR-520b, hsa-miR-520c-3p, hsa-miR-520d-3p, hsa-miR-520d-5p, hsa-miR-520e, hsa-miR-520f, hsa-miR-520g, hsa-miR-520h, hsa-miR-521, hsa-miR-522, hsa-miR-523, hsa-miR-524-3p, hsa-miR-524-5p, hsa-miR-525-3p, hsa-miR-525-5p, hsa-miR-526b, hsa-miR-526b*, hsa-miR-532-3p, hsa-miR-532-5p, hsa-miR-539, hsa-miR-541, hsa-miR-541*, hsa-miR-542-3p, hsa-miR-542-5p, hsa-miR-543, hsa-miR-544, hsa-miR-545, hsa-miR-545*, hsa-miR-548a-3p, hsa-miR-548a-5p, hsa-miR-548b-3p, hsa-miR-5486-5p, hsa-miR-548c-3p, hsa-miR-548c-5p, hsa-miR-548d-3p, hsa-miR-548d-5p, hsa-miR-548e, hsa-miR-548f, hsa-miR-548g, hsa-miR-548h, hsa-miR-548i, hsa-miR-548j, hsa-miR-548k, hsa-miR-5481, hsa-miR-548m, hsa-miR-548n, hsa-miR-548o, hsa-miR-548p, hsa-miR-549, hsa-miR-550, hsa-miR-550*, hsa-miR-551a, hsa-miR-551b, hsa-miR-551b*, hsa-miR-552, hsa-miR-553, hsa-miR-554, hsa-miR-555, hsa-miR-556-3p, hsa-miR-556-5p, hsa-miR-557, hsa-miR-558, hsa-miR-559, hsa-miR-561, hsa-miR-562, hsa-miR-563, hsa-miR-564, hsa-miR-566, hsa-miR-567, hsa-miR-568, hsa-miR-569, hsa-miR-570, hsa-miR-571, hsa-miR-572, hsa-miR-573, hsa-miR-574-3p, hsa-miR-574-5p, hsa-miR-575, hsa-miR-576-3p, hsa-miR-576-5p, hsa-miR-577, hsa-miR-578, hsa-miR-579, hsa-miR-580, hsa-miR-581, hsa-miR-582-3p, hsa-miR-582-5p, hsa-miR-583, hsa-miR-584, hsa-miR-585, hsa-miR-586, hsa-miR-587, hsa-miR-588, hsa-miR-589, hsa-miR-589*, hsa-miR-590-3p, hsa-miR-590-5p, hsa-miR-591, hsa-miR-592, hsa-miR-593, hsa-miR-593*, hsa-miR-595, hsa-miR-596, hsa-miR-597, hsa-miR-598, hsa-miR-599, hsa-miR-600, hsa-miR-601, hsa-miR-602, hsa-miR-603, hsa-miR-604, hsa-miR-605, hsa-miR-606, hsa-miR-607, hsa-miR-608, hsa-miR-609, hsa-miR-610, hsa-miR-611, hsa-miR-612, hsa-miR-613, hsa-miR-614, hsa-miR-615-3p, hsa-miR-615-5p, hsa-miR-616, hsa-miR-616*, hsa-miR-617, hsa-miR-618, hsa-miR-619, hsa-miR-620, hsa-miR-621, hsa-miR-622, hsa-miR-623, hsa-miR-624, hsa-miR-624*, hsa-miR-625, hsa-miR-625*, hsa-miR-626, hsa-miR-627, hsa-miR-628-3p, hsa-miR-628-5p, hsa-miR-629, hsa-miR-629*, hsa-miR-630, hsa-miR-631, hsa-miR-632, hsa-miR-633, hsa-miR-634, hsa-miR-635, hsa-miR-636, hsa-miR-637, hsa-miR-638, hsa-miR-639, hsa-miR-640, hsa-miR-641, hsa-miR-642, hsa-miR-643, hsa-miR-644, hsa-miR-645, hsa-miR-646, hsa-miR-647, hsa-miR-648, hsa-miR-649, hsa-miR-650, hsa-miR-651, hsa-miR-652, hsa-miR-653, hsa-miR-654-3p, hsa-miR-654-5p, hsa-miR-655, hsa-miR-656, hsa-miR-657, hsa-miR-658, hsa-miR-659, hsa-miR-660, hsa-miR-661, hsa-miR-662, hsa-miR-663, hsa-miR-663b, hsa-miR-664, hsa-miR-664*, hsa-miR-665, hsa-miR-668, hsa-miR-671-3p, hsa-miR-671-5p, hsa-miR-675, hsa-miR-7, hsa-miR-708, hsa-miR-708*, hsa-miR-7-1*, hsa-miR-7-2*, hsa-miR-720, hsa-miR-744, hsa-miR-744*, hsa-miR-758, hsa-miR-760, hsa-miR-765, hsa-miR-766, hsa-miR-767-3p, hsa-miR-767-5p, hsa-miR-768-3p, hsa-miR-768-5p, hsa-miR-769-3p, hsa-miR-769-5p, hsa-miR-770-5p, hsa-miR-802, hsa-miR-873, hsa-miR-874, hsa-miR-875-3p, hsa-miR-875-5p, hsa-miR-876-3p, hsa-miR-876-5p, hsa-miR-877, hsa-miR-877*, hsa-miR-885-3p, hsa-miR-885-5p, hsa-miR-886-3p, hsa-miR-886-5p, hsa-miR-887, hsa-miR-888, hsa-miR-888*, hsa-miR-889, hsa-miR-890, hsa-miR-891a, hsa-miR-891b, hsa-miR-892a, hsa-miR-892b, hsa-miR-9, hsa-miR-9*, hsa-miR-920, hsa-miR-921, hsa-miR-922, hsa-miR-923, hsa-miR-924, hsa-miR-92a, hsa-miR-92a-1*, hsa-miR-92a-2*, hsa-miR-92b, hsa-miR-92b*, hsa-miR-93, hsa-miR-93*, hsa-miR-933, hsa-miR-934, hsa-miR-935, hsa-miR-936, hsa-miR-937, hsa-miR-938, hsa-miR-939, hsa-miR-940, hsa-miR-941, hsa-miR-942, hsa-miR-943, hsa-miR-944, hsa-miR-95, hsa-miR-96, hsa-miR-96*, hsa-miR-98, hsa-miR-99a, hsa-miR-99a*, hsa-miR-99b, and hsa-miR-99b*. For example, miRNA targeting chromosome 8 open reading frame 72 (C9orf72) which expresses superoxide dismutase (SOD1), associated with amyotrophic lateral sclerosis (ALS) may be of interest.

A miRNA inhibits the function of the mRNAs it targets and, as a result, inhibits expression of the polypeptides encoded by the mRNAs. Thus, blocking (partially or totally) the activity of the miRNA (e.g., silencing the miRNA) can effectively induce, or restore, expression of a polypeptide whose expression is inhibited (derepress the polypeptide). In one embodiment, derepression of polypeptides encoded by mRNA targets of a miRNA is accomplished by inhibiting the miRNA activity in cells through any one of a variety of methods. For example, blocking the activity of a miRNA can be accomplished by hybridization with a small interfering nucleic acid (e.g., antisense oligonucleotide, miRNA sponge, TuD RNA) that is complementary, or substantially complementary to, the miRNA, thereby blocking interaction of the miRNA with its target mRNA. As used herein, a small interfering nucleic acid that is substantially complementary to a miRNA is one that is capable of hybridizing with a miRNA, and blocking the miRNA's activity. In some embodiments, a small interfering nucleic acid that is substantially complementary to a miRNA is a small interfering nucleic acid that is complementary with the miRNA at all but 1, 2, 3, 4, 5, 6, 7, 8, 9, 10, 11, 12, 13, 14, 15, 16, 17, or 18 bases. A “miRNA Inhibitor” is an agent that blocks miRNA function, expression and/or processing. For instance, these molecules include but are not limited to microRNA specific antisense, microRNA sponges, tough decoy RNAs (TuD RNAs) and microRNA oligonucleotides (double-stranded, hairpin, short oligonucleotides) that inhibit miRNA interaction with a Drosha complex.

Still other useful transgenes may include those encoding immunoglobulins which confer passive immunity to a pathogen. An “immunoglobulin molecule” is a protein containing the immunologically-active portions of an immunoglobulin heavy chain and immunoglobulin light chain covalently coupled together and capable of specifically combining with antigen. Immunoglobulin molecules are of any type (e.g., IgG, IgE, IgM, IgD, IgA and IgY), class (e.g., IgG1, IgG2, IgG3, IgG4, IgA1 and IgA2) or subclass. The terms “antibody” and “immunoglobulin” may be used interchangeably herein.

An “immunoglobulin heavy chain” is a polypeptide that contains at least a portion of the antigen binding domain of an immunoglobulin and at least a portion of a variable region of an immunoglobulin heavy chain or at least a portion of a constant region of an immunoglobulin heavy chain. Thus, the immunoglobulin derived heavy chain has significant regions of amino acid sequence homology with a member of the immunoglobulin gene superfamily. For example, the heavy chain in a Fab fragment is an immunoglobulin-derived heavy chain.

An “immunoglobulin light chain” is a polypeptide that contains at least a portion of the antigen binding domain of an immunoglobulin and at least a portion of the variable region or at least a portion of a constant region of an immunoglobulin light chain. Thus, the immunoglobulin-derived light chain has significant regions of amino acid homology with a member of the immunoglobulin gene superfamily.

An “immunoadhesin” is a chimeric, antibody-like molecule that combines the functional domain of a binding protein, usually a receptor, ligand, or cell-adhesion molecule, with immunoglobulin constant domains, usually including the hinge and Fc regions.

A “fragment antigen-binding” (Fab) fragment” is a region on an antibody that binds to antigens. It is composed of one constant and one variable domain of each of the heavy and the light chain.

The anti-pathogen construct is selected based on the causative agent (pathogen) for the disease against which protection is sought. These pathogens may be of viral, bacterial, or fungal origin, and may be used to prevent infection in humans against human disease, or in non-human mammals or other animals to prevent veterinary disease.

The rAAV may include genes encoding antibodies, and particularly neutralizing antibodies against a viral pathogen. Such anti-viral antibodies may include anti-influenza antibodies directed against one or more of Influenza A, Influenza B, and Influenza C. The type A viruses are the most virulent human pathogens. The serotypes of influenza A which have been associated with pandemics include, H1N1, which caused Spanish Flu in 1918, and Swine Flu in 2009; H2N2, which caused Asian Flu in 1957; H3N2, which caused Hong Kong Flu in 1968; H5N1, which caused Bird Flu in 2004; H7N7; H1N2; H9N2; H7N2; H7N3; and H10N7. Other target pathogenic viruses include, arenaviruses (including funin, machupo, and Lassa), filoviruses (including Marburg and Ebola), hantaviruses, picornoviridae (including rhinoviruses, echovirus), coronaviruses, paramyxovirus, morbillivirus, respiratory synctial virus, togavirus, coxsackievirus, JC virus, parvovirus B19, parainfluenza, adenoviruses, reoviruses, variola (Variola major (Smallpox)) and Vaccinia (Cowpox) from the poxvirus family, and varicella-zoster (pseudorabies). Viral hemorrhagic fevers are caused by members of the arenavirus family (Lassa fever) (which family is also associated with Lymphocytic choriomeningitis (LCM)), filovirus (ebola virus), and hantavirus (puremala). The members of picornavirus (a subfamily of rhinoviruses), are associated with the common cold in humans. The coronavirus family, which includes a number of non-human viruses such as infectious bronchitis virus (poultry), porcine transmissible gastroenteric virus (pig), porcine hemagglutinatin encephalomyelitis virus (pig), feline infectious peritonitis virus (cat), feline enteric coronavirus (cat), canine coronavirus (dog). The human respiratory coronaviruses, have been putatively associated with the common cold, non-A, B or C hepatitis, and sudden acute respiratory syndrome (SARS). The paramyxovirus family includes parainfluenza Virus Type 1, parainfluenza Virus Type 3, bovine parainfluenza Virus Type 3, rubulavirus (mumps virus, parainfluenza Virus Type 2, parainfluenza virus Type 4, Newcastle disease virus (chickens), rinderpest, morbillivirus, which includes measles and canine distemper, and pneumovirus, which includes respiratory syncytial virus (RSV). The parvovirus family includes feline parvovirus (feline enteritis), feline panleucopeniavirus, canine parvovirus, and porcine parvovirus. The adenovirus family includes viruses (EX, AD7, ARD, O.B.) which cause respiratory disease. Thus, in certain embodiments, a rAAV vector as described herein may be engineered to express an anti-ebola antibody, e.g., 2G4, 4G7, 13C6, an anti-influenza antibody, e.g., F16, CR8033, and anti-RSV antibody, e.g, palivizumab, motavizumab. A neutralizing antibody construct against a bacterial pathogen may also be selected for use in the present invention. In one embodiment, the neutralizing antibody construct is directed against the bacteria itself. In another embodiment, the neutralizing antibody construct is directed against a toxin produced by the bacteria. Examples of airborne bacterial pathogens include, e.g., Neisseria meningitidis (meningitis), Klebsiella pneumonia (pneumonia), Pseudomonas aeruginosa (pneumonia), Pseudomonas pseudomallei (pneumonia), Pseudomonas mallei (pneumonia), Acinetobacter (pneumonia), Moraxella catarrhalis, Moraxella lacunata, Alkaligenes, Cardiobacterium, Haemophilus influenzae (flu), Haemophilus parainfluenzae, Bordetella pertussis (whooping cough), Francisella tularensis (pneumonia/fever), Legionella pneumonia (Legionnaires disease), Chlamydia psittaci (pneumonia), Chlamydia pneumoniae (pneumonia), Mycobacterium tuberculosis (tuberculosis (TB)), Mycobacterium kansasii (TB), Mycobacterium avium (pneumonia), Nocardia asteroides (pneumonia), Bacillus anthracis (anthrax), Staphylococcus aureus (pneumonia), Streptococcus pyogenes (scarlet fever), Streptococcus pneumoniae (pneumonia), Corynebacteria diphtheria (diphtheria), Mycoplasma pneumoniae (pneumonia).

The rAAV may include genes encoding antibodies, and particularly neutralizing antibodies against a bacterial pathogen such as the causative agent of anthrax, a toxin produced by Bacillius anthracis. Neutralizing antibodies against protective agent (PA), one of the three peptides which form the toxoid, have been described. The other two polypeptides consist of lethal factor (LF) and edema factor (EF). Anti-PA neutralizing antibodies have been described as being effective in passively immunization against anthrax, See, e.g., U.S. Pat. No. 7,442,373; R. Sawada-Hirai et al, J Immune Based Ther Vaccines. 2004; 2: 5. (on-line 2004 May 12). Still other anti-anthrax toxin neutralizing antibodies have been described and/or may be generated. Similarly, neutralizing antibodies against other bacteria and/or bacterial toxins may be used to generate an AAV-delivered anti-pathogen construct as described herein.

Antibodies against infectious diseases may be caused by parasites or by fungi, including, e.g., Aspergillus species, Absidia corymbifera, Rhixpus stolonifer, Mucor plumbeaus, Cryptococcus neoformans, Histoplasm capsulatum, Blastomyces dermatitidis, Coccidioides immitis, Penicillium species, Micropolyspora faeni, Thermoactinomyces vulgaris, Alternaria alternate, Cladosporium species, Helminthosporium, and Stachybotrys species.

The rAAV may include genes encoding antibodies, and particularly neutralizing antibodies, against pathogenic factors of diseases such as Alzheimer's disease (AD), Parkinson's disease (PD), GBA-associated-Parkinson's disease (GBA-PD), Rheumatoid arthritis (RA), Irritable bowel syndrome (IBS), chronic obstructive pulmonary disease (COPD), cancers, tumors, systemic sclerosis, asthma and other diseases. Such antibodies may be, without limitation, e.g., alpha-synuclein, anti-vascular endothelial growth factor (VEGF) (anti-VEGF), anti-VEGFA, anti-PD-1, anti-PDL1, anti-CTLA-4, anti-TNF-alpha, anti-IL-17, anti-IL-23, anti-IL-21, anti-IL-6, anti-IL-6 receptor, anti-IL-5, anti-IL-7, anti-Factor XII, anti-IL-2, anti-HIV, anti-IgE, anti-tumour necrosis factor receptor-1 (TNFR1), anti-notch 2/3, anti-notch 1, anti-OX40, anti-erb-b2 receptor tyrosine kinase 3 (ErbB3), anti-ErbB2, anti-beta cell maturation antigen, anti-B lymphocyte stimulator, anti-CD20, anti-HER2, anti-granulocyte macrophage colony-stimulating factor, anti-oncostatin M (OSM), anti-lymphocyte activation gene 3 (LAG3) protein, anti-CCL20, anti-serum amyloid P component (SAP), anti-prolyl hydroxylase inhibitor, anti-CD38, anti-glycoprotein IIb/IIIa, anti-CD52, anti-CD30, anti-IL-1beta, anti-epidermal growth factor receptor, anti-CD25, anti-RANK ligand, anti-complement system protein C5, anti-CD11a, anti-CD3 receptor, anti-alpha-4 (α4) integrin, anti-RSV F protein, and anti-integrin α₄β₇. Still other pathogens and diseases will be apparent to one of skill in the art. Other suitable antibodies may include those useful for treating Alzheimer's Disease, such as, e.g., anti-beta-amyloid (e.g., crenezumab, solanezumab, aducanumab), anti-beta-amyloid fibril, anti-beta-amyloid plaques, anti-tau, a bapineuzamab, among others. Other suitable antibodies for treating a variety of indications include those described, e.g., in PCT/US2016/058968, filed 27 Oct. 2016, published as WO 2017/075119A1.

Reduction and/or modulation of expression of a gene is particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable therapeutic polypeptides and proteins include those which may be useful for treating individuals suffering from autoimmune diseases and disorders by conferring a broad based protective immune response against targets that are associated with autoimmunity including cell receptors and cells which produce self-directed antibodies. T cell mediated autoimmune diseases include Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

Alternatively, or in addition, the vectors may contain AAV sequences of the invention and a transgene encoding a peptide, polypeptide or protein which induces an immune response to a selected immunogen. For example, immunogens may be selected from a variety of viral families. Example of desirable viral families against which an immune response would be desirable include, the picornavirus family, which includes the genera rhinoviruses, which are responsible for about 50% of cases of the common cold; the genera enteroviruses, which include polioviruses, coxsackieviruses, echoviruses, and human enteroviruses such as hepatitis A virus; and the genera apthoviruses, which are responsible for foot and mouth diseases, primarily in non-human animals. Within the picornavirus family of viruses, target antigens include the VP1, VP2, VP3, VP4, and VPG. Another viral family includes the calcivirus family, which encompasses the Norwalk group of viruses, which are an important causative agent of epidemic gastroenteritis. Still another viral family desirable for use in targeting antigens for inducing immune responses in humans and non-human animals is the togavirus family, which includes the genera alphavirus, which include Sindbis viruses, RossRiver virus, and Venezuelan, Eastern & Western Equine encephalitis, and rubivirus, including Rubella virus. The flaviviridae family includes dengue, yellow fever, Japanese encephalitis, St. Louis encephalitis and tick borne encephalitis viruses. Other target antigens may be generated from the Hepatitis C or the coronavirus family, which includes a number of non-human viruses such as infectious bronchitis virus (poultry), porcine transmissible gastroenteric virus (pig), porcine hemagglutinating encephalomyelitis virus (pig), feline infectious peritonitis virus (cats), feline enteric coronavirus (cat), canine coronavirus (dog), and human respiratory coronaviruses, which may cause the common cold and/or non-A, B or C hepatitis. Within the coronavirus family, target antigens include the E1 (also called M or matrix protein), E2 (also called S or Spike protein), E3 (also called HE or hemagglutin-elterose) glycoprotein (not present in all coronaviruses), or N (nucleocapsid). Still other antigens may be targeted against the rhabdovirus family, which includes the genera vesiculovirus (e.g., Vesicular Stomatitis Virus), and the general lyssavirus (e.g., rabies). Within the rhabdovirus family, suitable antigens may be derived from the G protein or the N protein. The family filoviridae, which includes hemorrhagic fever viruses such as Marburg and Ebola virus may be a suitable source of antigens. The paramyxovirus family includes parainfluenza Virus Type 1, parainfluenza Virus Type 3, bovine parainfluenza Virus Type 3, rubulavirus (mumps virus, parainfluenza Virus Type 2, parainfluenza virus Type 4, Newcastle disease virus (chickens), rinderpest, morbillivirus, which includes measles and canine distemper, and pneumovirus, which includes respiratory syncytial virus. The influenza virus is classified within the family orthomyxovirus and is a suitable source of antigen (e.g., the HA protein, the N1 protein). The bunyavirus family includes the genera bunyavirus (California encephalitis, La Crosse), phlebovirus (Rift Valley Fever), hantavirus (puremala is a hemahagin fever virus), nairovirus (Nairobi sheep disease) and various unassigned bungaviruses. The arenavirus family provides a source of antigens against LCM and Lassa fever virus. The reovirus family includes the genera reovirus, rotavirus (which causes acute gastroenteritis in children), orbiviruses, and cultivirus (Colorado Tick fever, Lebombo (humans), equine encephalosis, blue tongue).

The retrovirus family includes the sub-family oncorivirinal which encompasses such human and veterinary diseases as feline leukemia virus, HTLVI and HTLVII, lentivirinal (which includes human immunodeficiency virus (HIV), simian immunodeficiency virus (SIV), feline immunodeficiency virus (FIV), equine infectious anemia virus, and spumavirinal). Between the HIV and SIV, many suitable antigens have been described and can readily be selected. Examples of suitable HIV and SIV antigens include, without limitation the gag, pol, Vif, Vpx, VPR, Env, Tat and Rev proteins, as well as various fragments thereof. In addition, a variety of modifications to these antigens have been described. Suitable antigens for this purpose are known to those of skill in the art. For example, one may select a sequence encoding the gag, pol, Vif, and Vpr, Env, Tat and Rev, amongst other proteins. See, e.g., the modified gag protein which is described in U.S. Pat. No. 5,972,596. See, also, the HIV and SIV proteins described in D. H. Barouch et al, J. Virol., 75(5):2462-2467 (March 2001), and R. R. Amara, et al, Science, 292:69-74 (6 Apr. 2001). These proteins or subunits thereof may be delivered alone, or in combination via separate vectors or from a single vector.

The papovavirus family includes the sub-family polyomaviruses (BKU and JCU viruses) and the sub-family papillomavirus (associated with cancers or malignant progression of papilloma). The adenovirus family includes viruses (EX, AD7, ARD, O.B.) which cause respiratory disease and/or enteritis. The parvovirus family feline parvovirus (feline enteritis), feline panleucopeniavirus, canine parvovirus, and porcine parvovirus. The herpesvirus family includes the sub-family alphaherpesvirinae, which encompasses the genera simplexvirus (HSVI, HSVII), varicellovirus (pseudorabies, varicella zoster) and the sub-family betaherpesvirinae, which includes the genera cytomegalovirus (HCMV, muromegalovirus) and the sub-family gammaherpesvirinae, which includes the genera lymphocryptovirus, EBV (Burkitts lymphoma), infectious rhinotracheitis, Marek's disease virus, and rhadinovirus. The poxvirus family includes the sub-family chordopoxvirinae, which encompasses the genera orthopoxvirus (Variola (Smallpox) and Vaccinia (Cowpox)), parapoxvirus, avipoxvirus, capripoxvirus, leporipoxvirus, suipoxvirus, and the sub-family entomopoxvirinae. The hepadnavirus family includes the Hepatitis B virus. One unclassified virus which may be suitable source of antigens is the Hepatitis delta virus. Still other viral sources may include avian infectious bursal disease virus and porcine respiratory and reproductive syndrome virus. The alphavirus family includes equine arteritis virus and various Encephalitis viruses.

The rAAV may also deliver a sequence encoding immunogens which are useful to immunize a human or non-human animal against other pathogens including bacteria, fungi, parasitic microorganisms or multicellular parasites which infect human and non-human vertebrates, or from a cancer cell or tumor cell. Examples of bacterial pathogens include pathogenic gram-positive cocci include pneumococci; staphylococci; and streptococci. Pathogenic gram-negative cocci include meningococcus; gonococcus. Pathogenic enteric gram-negative bacilli include enterobacteriaceae; pseudomonas, acinetobacteria and eikenella; melioidosis; salmonella; shigella; haemophilus; moraxella; H. ducreyi (which causes chancroid); brucella; Franisella tularensis (which causes tularemia); yersinia (pasteurella); streptobacillus moniliformis and spirillum; Gram-positive bacilli include Listeria monocytogenes; erysipelothrix rhusiopathiae; Corynebacterium diphtheria (diphtheria); cholera; B. anthracis (anthrax); donovanosis (granuloma inguinale); and bartonellosis. Diseases caused by pathogenic anaerobic bacteria include tetanus; botulism; other clostridia; tuberculosis; leprosy; and other mycobacteria. Pathogenic spirochetal diseases include syphilis; treponematoses: yaws, pinta and endemic syphilis; and leptospirosis. Other infections caused by higher pathogen bacteria and pathogenic fungi include actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma and chromomycosis; and dermatophytosis. Rickettsial infections include Typhus fever, Rocky Mountain spotted fever, Q fever, and Rickettsial pox. Examples of mycoplasma and chlamydial infections include: Mycoplasma pneumoniae; lymphogranuloma venereum; psittacosis; and perinatal chlamydial infections. Pathogenic eukaryotes encompass pathogenic protozoans and helminths and infections produced thereby include: amebiasis; malaria; leishmaniasis; trypanosomiasis; toxoplasmosis; Pneumocystis carinii; Trichans; Toxoplasma gondii; babesiosis; giardiasis; trichinosis; filariasis; schistosomiasis; nematodes; trematodes or flukes; and cestode (tapeworm) infections.

Many of these organisms and/or toxins produced thereby have been identified by the Centers for Disease Control [(CDC), Department of Health and Human Services, USA], as agents which have potential for use in biological attacks. For example, some of these biological agents, include, Bacillus anthracis (anthrax), Clostridium botulinum and its toxin (botulism), Yersinia pestis (plague), variola major (smallpox), Francisella tularensis (tularemia), and viral hemorrhagic fever, all of which are currently classified as Category A agents; Coxiella burnetti (Q fever); Brucella species (brucellosis), Burkholderia mallei (glanders), Ricinus communis and its toxin (ricin toxin), Clostridium perfringens and its toxin (epsilon toxin), Staphylococcus species and their toxins (enterotoxin B), all of which are currently classified as Category B agents; and Nipan virus and hantaviruses, which are currently classified as Category C agents. In addition, other organisms, which are so classified or differently classified, may be identified and/or used for such a purpose in the future. It will be readily understood that the viral vectors and other constructs described herein are useful to deliver antigens from these organisms, viruses, their toxins or other by-products, which will prevent and/or treat infection or other adverse reactions with these biological agents.

Administration of the vectors of the invention to deliver immunogens against the variable region of the T cells elicit an immune response including CTLs to eliminate those T cells. In rheumatoid arthritis (RA), several specific variable regions of T cell receptors (TCRs) which are involved in the disease have been characterized. These TCRs include V-3, V-14, V-17 and Va-17. Thus, delivery of a nucleic acid sequence that encodes at least one of these polypeptides will elicit an immune response that will target T cells involved in RA. In multiple sclerosis (MS), several specific variable regions of TCRs which are involved in the disease have been characterized. These TCRs include V-7 and Va-10. Thus, delivery of a nucleic acid sequence that encodes at least one of these polypeptides will elicit an immune response that will target T cells involved in MS. In scleroderma, several specific variable regions of TCRs which are involved in the disease have been characterized. These TCRs include V-6, V-8, V-14 and Va-16, Va-3C, Va-7, Va-14, Va-15, Va-16, Va-28 and Va-12. Thus, delivery of a nucleic acid molecule that encodes at least one of these polypeptides will elicit an immune response that will target T cells involved in scleroderma.

In one embodiment, the transgene is selected to provide optogenetic therapy. In optogenetic therapy, artificial photoreceptors are constructed by gene delivery of light-activated channels or pumps to surviving cell types in the remaining retinal circuit. This is particularly useful for patients who have lost a significant amount of photoreceptor function, but whose bipolar cell circuitry to ganglion cells and optic nerve remains intact. In one embodiment, the heterologous nucleic acid sequence (transgene) is an opsin. The opsin sequence can be derived from any suitable single- or multicellular-organism, including human, algae and bacteria. In one embodiment, the opsin is rhodopsin, photopsin, L/M wavelength (red/green)-opsin, or short wavelength (S) opsin (blue). In another embodiment, the opsin is channel rhodopsin or halorhodopsin.

In another embodiment, the transgene is selected for use in gene augmentation therapy, i.e., to provide replacement copy of a gene that is missing or defective. In this embodiment, the transgene may be readily selected by one of skill in the art to provide the necessary replacement gene. In one embodiment, the missing/defective gene is related to an ocular disorder. In another embodiment, the transgene is NYX, GRM6, TRPM1L or GPR179 and the ocular disorder is Congenital Stationary Night Blindness. See, e.g., Zeitz et al, Am J Hum Genet. 2013 Jan. 10; 92(1):67-75. Epub 2012 Dec. 13 which is incorporated herein by reference. In another embodiment, the transgene is RPGR. In another embodiment, the gene is Rab escort protein 1 (REP-1) encoded by CHM, associated with choroideremia.

In another embodiment, the transgene is selected for use in gene suppression therapy, i.e., expression of one or more native genes is interrupted or suppressed at transcriptional or translational levels. This can be accomplished using short hairpin RNA (shRNA) or other techniques well known in the art. See, e.g., Sun et al, Int J Cancer, 2010 Feb. 1; 126(3):764-74 and O'Reilly M, et al. Am J Hum Genet. 2007 July; 81(1):127-35, which are incorporated herein by reference. In this embodiment, the transgene may be readily selected by one of skill in the art based upon the gene which is desired to be silenced.

In another embodiment, the transgene comprises more than one transgene. This may be accomplished using a single vector carrying two or more heterologous sequences, or using two or more rAAV each carrying one or more heterologous sequences. In one embodiment, the rAAV is used for gene suppression (or knockdown) and gene augmentation co-therapy. In knockdown/augmentation co-therapy, the defective copy of the gene of interest is silenced and a non-mutated copy is supplied. In one embodiment, this is accomplished using two or more co-administered vectors. See, Millington-Ward et al, Molecular Therapy, April 2011, 19(4):642-649 which is incorporated herein by reference. The transgenes may be readily selected by one of skill in the art based on the desired result.

In another embodiment, the transgene is selected for use in gene correction therapy. This may be accomplished using, e.g., a zinc-finger nuclease (ZFN)-induced DNA double-strand break in conjunction with an exogenous DNA donor substrate. See, e.g., Ellis et al, Gene Therapy (epub January 2012) 20:35-42 which is incorporated herein by reference. In one embodiment, the transgene encodes a nuclease selected from a meganuclease, a zinc finger nuclease, a transcription activator-like (TAL) effector nuclease (TALEN), and a clustered, regularly interspaced short palindromic repeat (CRISPR)/endonuclease (Cas9, Cpf1, etc). Examples of suitable meganucleases are described, e.g., in U.S. Pat. Nos. 8,445,251; 9,340,777; 9,434,931; 9,683,257, and WO 2018/195449. Other suitable enzymes include nuclease-inactive S. pyogenes CRISPR/Cas9 that can bind RNA in a nucleic-acid-programmed manner (Nelles et al, Programmable RNA Tracking in Live Cells with CRISPR/Cas9, Cell, 165(2):P488-96 (April 2016)), and base editors (e.g., Levy et al. Cytosine and adenine base editing of the brain, liver, retina, heart and skeletal muscle of mice via adeno-associated viruses, Nature Biomedical Engineering, 4, 97-110 (January 2020)). In certain embodiments, the nuclease is not a zinc finger nuclease. In certain embodiments, the nuclease is not a CRISPR-associated nuclease. In certain embodiments, the nuclease is not a TALEN. In one embodiment, the nuclease is not a meganuclease. In certain embodiments, the nuclease is a member of the I-CreI family of homing endonucleases which recognizes and cuts a 22 base pair recognition sequence. See, e.g., WO 2009/059195. Methods for rationally-designing homing endonucleases were described which are capable of comprehensively redesigning ICreI and other homing endonucleases to target widely-divergent DNA sites, including sites in mammalian, yeast, plant, bacterial, and viral genomes (WO 2007/047859).

In certain embodiments, a rAAV-based gene editing nuclease system is provided herein. The gene editing nuclease targets sites in a disease-associated gene, i.e., gene of interest.

In certain embodiments, the AAV-based gene editing nuclease system comprises an rAAV comprising an AAV capsid and enclosed therein a vector genome, wherein the vector genome comprising AAV 5′ inverted terminal repeats (ITR), an expression cassette comprising a nucleic acid sequence encoding a gene editing nuclease which recognizes and cleaves a recognition site in a gene of interest, wherein said gene editing nuclease coding sequence is operably linked to expression control sequences which direct expression thereof in a cell comprising the gene of interest, and an AAV 3′ ITR.

Provided herein also is a method of treatment using an rAAV-based gene editing nuclease system.

In some embodiments, the rAAV-based gene editing meganuclease system is used for treating diseases, disorders, syndrome and/or conditions. In some embodiments, the gene editing nuclease is targeted to a gene of interest, wherein the gene of interest has one or more genetic mutation, deletion, insertion, and/or a defect which is associated with and/or implicated in a disease, disorder, syndrome and/or conditions. In some embodiments, the disorder is selected but not limited to cardiovascular, hepatic, endocrine or metabolic, musculoskeletal, neurological, and/or renal disorders.

In certain embodiments, the indicated cardiovascular diseases, disorders, syndrome and/or conditions include, but not limited to, cardiovascular disease (associated lysophosphatidic acid, lipoprotein (a), or angiopoietin-like 3 (ANGPTL3), or apolipoprotein C-III (APOC3) encoding genes), block coagulation, thrombosis, end stage renal disease, clotting disorders (associated with Factor XI (F11) encoding gene), hypertension (angiotensinogen (AGT) encoding gene), and heart failure (angiotensinogen (AGT) encoding gene).

In certain embodiments, the indicated hepatic diseases, disorders, syndrome and/or conditions include, but not limited to, idiopathic pulmonary fibrosis (associated with SERPINH1/Hsp47 gene), liver disease (associated with hydroxysteroid 17-beta dehydrogenase 13 (HSD17B13) encoding gene, non-alcoholic steatohepatitis (NASH) (associated with diacylglycerol O-acyltransferase-2 (DGAT2), hydroxysteroid 17-Beta Dehydrogenase 13 (HSD17B13), or patatin-like phospholipase domain-containing 3 (PNPLA3) encoding genes), and alcohol use disorder (associated with aldehyde dehydrogenase 2 (ALDH2) encoding gene).

In certain embodiments, the indicated musculoskeletal diseases, disorders, syndrome and/or conditions include, but not limited to, muscular dystrophy (associated with dystrophin, or integrin alpha(4) (VLA-4) (CD49D) encoding genes), Duchene muscular dystrophy (DMD) (associated with dystrophin (DMD) gene), centronuclear myopathy (associated with dynamin 2 (DNM2) encoding gene), and myotonic dystrophy (DM1) (associated with myotonic dystrophy protein kinase (DMPK) encoding gene).

In certain embodiments, the indicated endocrine or metabolic diseases, disorders, syndrome and/or conditions include, but not limited to, hypertriglyceridemia (associated with apolipoprotein C-III (APOC3), or angiopoietin-like 3 (ANGPTL3) encoding genes), lipodystrophy, hyperlipidemia (associated with apolipoprotein C-III (APOC3) encoding gene), hypercholesterolemia (associated with apolipoprotein B-100 (APOB-100), proprotein convertase subtilisin kexin type 9 (PCSK9)), or amyloidosis (associated with transthyretin (TTR) encoding gene), porphyria (associated with aminolevulinate synthase-1 (ALAS-1) encoding gene), neuropathy (associated with transthyretin (TTR) encoding gene), primary hyperoxaluria type 1 (associated with glycolate oxidase encoding gene), diabetes (associated with Glucagon receptor (GCGR) encoding gene), acromegaly (growth hormone receptor (GHR) encoding gene), alpha-1 antitrypsin deficiency (AATD) (associated with alpha-1 antitrypsin (AAT) encoding gene), propionic acidemia (propionyl-CoA carboxylase (PCCA/PCCB) encoding gene), glycogen storage disease type III (GDSIII) (associated with glycogen debranching enzyme (GSDIII) encoding gene), cardiometabolic disease (associated with asialoglycoprotein (ASGPR), hydroxyacid Oxidase 1 (HAG1), or alpha-1-antitrypsin (SERPINA1) encoding genes), methylmalonic acidemia (MMA) (associated with methylmalonyl CoA mutase (MMUT), cob(I)alamin adenosyltransferase (MMAA or MMAB), methylmalonyl-CoA epimerase (MCEE), LMBR1 domain containing 1 (LMBRD1), or ATP-binding cassette subfamily D member 4 (ABCD4) encoding genes), glycogen storage disease type 1a (associated with Glucose-6-phosphatase catalytic subunit-related protein (G6PC) encoding gene), and phenylketonuria (PKU) (associated with phenylalanine hydroxylase (PAH) encoding gene).

In certain embodiments, the indicated neurological diseases, disorders, syndrome and/or conditions include, but not limited to, spinal muscular atrophy (SMA) (associated with survival motor neuron protein (SMN2) gene), amyotrophic lateral sclerosis (ALS) (superoxide dismutase type 1 (SOD1), FUS RNA binding protein (FUS), microRNA-155, chromosome 9 open reading frame 72 (C9orf72), or ataxin-2 (ATXN2) genes), Huntington disease (associated with huntingtin (HTT) gene), hATTR polyneuropathy (associated with transthyretin (TTR) gene), Alzheimer's disease (associated with MAP-tau (MAPT) gene), Multiple System Atrophy (associated with alpha-synuclein (SNCA)), Parkinson's disease (associated with alpha-synuclein (SNCA), leucine rich repeat kinase 2 (LRRK2) genes), centronuclear myopathy (associated with dynamin 2 (DNM2) gene), Angelman syndrome (associated with ubiquitin protein ligase E3A (UBE3A) gene), epilepsy (associated with glycogen synthase 1 (GYS1) gene), Dravet Syndrome (associated with sodium voltage-gated channel alpha subunit 1 (SNC1A) gene), Leukodystrophy (associated with glial fibrillary acidic protein (GFAP) gene), prion disease (associated with prion protein (PRNP) gene), and Hereditary cerebral hemorrhage with amyloidosis-Dutch type (HCHWA-D) (associated with amyloid beta precursor protein (APP) gene).

In certain embodiments, the indicated renal diseases, disorders, syndrome and/or conditions include, but not limited to, Glomerulonephritis (IgA Nephropathy) (associated with complement factor B encoding gene), Alport syndrome (associated with proteins in the PPARα signaling pathway), and neuropathy (associated with apolipoprotein L1 (APOL1) encoding gene) or an APOL1-associated chronic kidney disease.

In certain embodiments, the gene editing nuclease is targeted to the gene of interest, wherein the gene of interest includes but not limited to lysophosphatidic acid encoding gene, lipoprotein (a) encoding gene, ANGPTL3, APOC3, F11, AGT, SERPINH1/Hsp47, HSD17B13, DGAT2, PNPLA3, ALDH2, DMD, VLA-4, DNM2DM1, DMPK, APOC3, ANGPTL3, APOB-100, PCSK9, TTR, ALAS-1, glycolate oxidase encoding gene, GCGR, GHR, AATD, AAT, PCCA, PCCB, GDSIII, ASGPR, HAO1, SERPINA1, MMA, MMUT, MMAA, MMAB, MCEE, LMBRD1, ABCD4, G6PC, PAH. SMN2, SOD1, FUS, C9orf72, ATXN2, HTT, MAPT, SNCA, LRRK2, UBE3A, GYS1, SNC1A, GFAP, PRNP, APP, complement factor B encoding gene, APOL1, AAS1, SLC25A13 genes.

Suitable gene editing targets include, e.g., liver-expressed genes such as, without limitation, proprotein convertase subtilisin/kexin type 9 (PCSK9) (cholesterol related disorders), transthyretin (TTR) (transthyretin amyloidosis), HAO, apolipoprotein C-III (APOC3), Factor VIII, Factor IX, low density lipoprotein receptor (LDLr), lipoprotein lipase (LPL) (Lipoprotein Lipase Deficiency), lecithin-cholesterol acyltransferase (LCAT), ornithine transcarbamylase (OTC), carnosinase (CN1), sphingomyelin phosphodiesterase (SMPD1) (Niemann-Pick disease), hypoxanthine-guanine phosphoribosyltransferase (HGPRT), branched-chain alpha-keto acid dehydrogenase complex (BCKDC) (maple syrup urine disease), erythropoietin (EPO), Carbamyl Phosphate Synthetase (CPS1), N-Acetylglutamate Synthetase (NAGS), Argininosuccinic Acid Synthetase (Citrullinemia), Argininosuccinate Lyase (ASL) (Argininosuccinic Aciduria), and Arginase (AG).

Other gene editing targets may include, e.g., hydroxymethylbilane synthase (HMBS), carbamoyl synthetase I, ornithine transcarbamylase (OTC), arginosuccinate synthetase, alpha 1 anti-trypsin (A1AT), aaporginosuccinate lyase (ASL) for treatment of argunosuccinate lyase deficiency, arginase, fumarylacetate hydrolase, phenylalanine hydroxylase, alpha-1 antitrypsin, rhesus alpha-fetoprotein (AFP), rhesus chorionic gonadotrophin (CG), glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase (MUT), glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding β-glucuronidase (GUSB)). In another example, the gene product is ubiquitin protein ligase. glucose-6-phosphatase, associated with glycogen storage disease or deficiency type 1A (GSD1), phosphoenolpyruvate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase (PAH), associated with phenylketonuria (PKU); gene products associated with Primary Hyperoxaluria Type 1 including Hydroxyacid Oxidase 1 (GO/HAO1) and AGXT, branched chain alpha-ketoacid dehydrogenase, including BCKDH, BCKDH-E2, BAKDH-Ela, and BAKDH-E1b, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASS1), associated with citrullinemia; lecithin-cholesterol acyltransferase (LCAT) deficiency; amethylmalonic acidemia (MMA); NPC1 associated with Niemann-Pick disease, type C1); propionic academia (PA); TTR associated with Transthyretin (TTR)-related Hereditary Amyloidosis; low density lipoprotein receptor (LDLR) protein, associated with familial hypercholesterolemia (FH), LDLR variant, such as those described in WO 2015/164778; PCSK9; ApoE and ApoC proteins, associated with dementia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); beta-galactosidase (GLB1) associated with GM1 gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosucesinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; α-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

In one embodiment, the capsids described herein are useful in the CRISPR-Cas dual vector system described in US Published Patent Application 2018/0110877, filed Apr. 26, 2018, each of which is incorporated herein by reference. The capsids are also useful for delivery homing endonucleases or other meganucleases.

In another embodiment, the transgenes useful herein include reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

In certain embodiments, in addition to the transgene coding sequence, another non-AAV coding sequence may be included, e.g., a peptide, polypeptide, protein, functional RNA molecule (e.g., miRNA, miRNA inhibitor) or other gene product, of interest. Useful gene products may include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

These above coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

Desirably, the transgene encodes a product which is useful in biology and medicine, such as proteins, peptides, RNA, enzymes, or catalytic RNAs. Desirable RNA molecules include shRNA, tRNA, dsRNA, ribosomal RNA, catalytic RNAs, and antisense RNAs. One example of a useful RNA sequence is a sequence which extinguishes expression of a targeted nucleic acid sequence in a target cell.

Regulatory sequences include conventional control elements which are operably linked to the transgene in a manner which permits its transcription, translation and/or expression in a cell transfected with the vector or infected with the virus produced as described herein. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters, are known in the art and may be utilized.

The regulatory sequences useful in the constructs provided herein may also contain an intron, desirably located between the promoter/enhancer sequence and the gene. One desirable intron sequence is derived from SV-40, and is a 100 bp mini-intron splice donor/splice acceptor referred to as SD-SA. Another suitable sequence includes the woodchuck hepatitis virus post-transcriptional element. (See, e.g., L. Wang and I. Verma, 1999 Proc. Natl. Acad. Sci., USA, 96:3906-3910). PolyA signals may be derived from many suitable species, including, without limitation SV-40, human and bovine.

Another regulatory component of the rAAV useful in the methods described herein is an internal ribosome entry site (IRES). An IRES sequence, or other suitable systems, may be used to produce more than one polypeptide from a single gene transcript. An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. Preferably, the IRES is located 3′ to the transgene in the rAAV vector.

In certain embodiments, the vector genome comprises a promoter (or a functional fragment of a promoter). The selection of the promoter to be employed in the rAAV may be made from among a wide number of constitutive or inducible promoters that can express the selected transgene in the desired target cell. In one embodiment, the target cell is an ocular cell. The promoter may be derived from any species, including human. Desirably, in one embodiment, the promoter is “cell specific”. The term “cell-specific” means that the particular promoter selected for the recombinant vector can direct expression of the selected transgene in a particular cell tissue. In one embodiment, the promoter is specific for expression of the transgene in muscle cells. In another embodiment, the promoter is specific for expression in lung. In another embodiment, the promoter is specific for expression of the transgene in liver cells. In another embodiment, the promoter is specific for expression of the transgene in airway epithelium. In another embodiment, the promoter is specific for expression of the transgene in neurons. In another embodiment, the promoter is specific for expression of the transgene in heart.

The vector genome typically contains a promoter sequence as part of the expression control sequences, e.g., located between the selected 5′ ITR sequence and the immunoglobulin construct coding sequence. In one embodiment, expression in liver is desirable. Thus, in one embodiment, a liver-specific promoter is used. Examples of liver-specific promoters may include, e.g., thyroid hormone-binding globulin (TBG), albumin, Miyatake et al., (1997) J. Virol., 71:5124 32; hepatitis B virus core promoter, Sandig et al., (1996) Gene Ther., 3:1002 9; or human alpha 1-antitrypsin, phosphoenolpyruvate carboxykinase (PECK), or alpha fetoprotein (AFP), Arbuthnot et al., (1996) Hum. Gene Ther., 7:1503 14). Tissue specific promoters, constitutive promoters, regulatable promoters [see, e.g., WO 2011/126808 and WO 2013/04943], or a promoter responsive to physiologic cues may be used may be utilized in the vectors described herein. In another embodiment, expression in muscle is desirable. Thus, in one embodiment, a muscle-specific promoter is used. In one embodiment, the promoter is an MCK based promoter, such as the dMCK (509-bp) or tMCK (720-bp) promoters (see, e.g., Wang et al, Gene Ther. 2008 November; 15(22):1489-99. doi: 10.1038/gt.2008.104. Epub 2008 Jun. 19, which is incorporated herein by reference). Another useful promoter is the SPc5-12 promoter (see Rasowo et al, European Scientific Journal June 2014 edition vol. 10, No. 18, which is incorporated herein by reference). In certain embodiments, a promoter specific for the eye or a subpart thereof (e.g., retina) may be selected.

In one embodiment, the promoter is a CMV promoter. In another embodiment, the promoter is a TBG promoter. In another embodiment, a CB7 promoter is used. CB7 is a chicken β-actin promoter with cytomegalovirus enhancer elements. Alternatively, other liver-specific promoters may be used [see, e.g., The Liver Specific Gene Promoter Database, Cold Spring Harbor, rulai.schl.edu/LSPD, alpha 1 anti-trypsin (A1AT); human albumin Miyatake et al., J. Virol., 71:5124 32 (1997), humA1b; and hepatitis B virus core promoter, Sandig et al., Gene Ther., 3:1002 9 (1996)]. TTR minimal enhancer/promoter, alpha-antitrypsin promoter, LSP (845 nt)25 (requires intron-less scAAV).

The promoter(s) can be selected from different sources, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter.

The vector genome may contain at least one enhancer, i.e., CMV enhancer. Still other enhancer elements may include, e.g., an apolipoprotein enhancer, a zebrafish enhancer, a GFAP enhancer element, and brain specific enhancers such as described in WO 2013/1555222, woodchuck post hepatitis post-transcriptional regulatory element. Additionally, or alternatively, other, e.g., the hybrid human cytomegalovirus (HCMV)-immediate early (IE)-PDGR promoter or other promoter—enhancer elements may be selected. Other enhancer sequences useful herein include the IRBP enhancer (Nicoud 2007, J Gene Med. 2007 December; 9(12):1015-23), immediate early cytomegalovirus enhancer, one derived from an immunoglobulin gene or SV40 enhancer, the cis-acting element identified in the mouse proximal promoter, etc.

In addition to a promoter, a vector genome may contain other appropriate transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A variety of suitable polyA are known. In one example, the polyA is rabbit beta globin, such as the 127 bp rabbit beta-globin polyadenylation signal (GenBank #V00882.1). In other embodiments, an SV40 polyA signal is selected. Still other suitable polyA sequences may be selected. In certain embodiments, an intron is included. One suitable intron is a chicken beta-actin intron. In one embodiment, the intron is 875 bp (GenBank #X00182.1). In another embodiment, a chimeric intron available from Promega is used. However, other suitable introns may be selected. In one embodiment, spacers are included such that the vector genome is approximately the same size as the native AAV vector genome (e.g., between 4.1 and 5.2 kb). In one embodiment, spacers are included such that the vector genome is approximately 4.7 kb. See, Wu et al, Effect of Genome Size on AAV Vector Packaging, Mol Ther. 2010 January; 18(1): 80-86, which is incorporated herein by reference.

In certain embodiments, the vector genome further comprises dorsal root ganglion (drg)-specific miRNA detargeting sequences operably linked to the transgene coding sequence. In certain embodiments, the tandem miRNA target sequences are continuous or are separated by a spacer of 1 to 10 nucleic acids, wherein said spacer is not an miRNA target sequence. In certain embodiments, there are at least two drg-specific miRNA sequences located at 3′ to a functional transgene coding sequence. In certain embodiments, the start of the first of the at least two drg-specific miRNA tandem repeats is within 20 nucleotides from the 3′ end of the transgene coding sequence. In certain embodiments, the start of the first of the at least two drg-specific miRNA tandem repeats is at least 100 nucleotides from the 3′ end of the functional transgene coding sequence. In certain embodiments, the miRNA tandem repeats comprise 200 to 1200 nucleotides in length. In certain embodiments, there are at least two drg-specific miRNA target sequences located at 5′ to the functional transgene coding sequence. In certain embodiments, at least two drg-specific miRNA target sequences are located in both 5′ and 3′ to the functional transgene coding sequence. See International Patent Application No. PCT/US19/67872, filed Dec. 20, 2019, U.S. Provisional Patent Application No. 63/023,594, filed May 12, 2020, International Patent Application No. PCT/US2021/032003, published as WO 2021/231579 on Nov. 18, 2021, which is incorporated herein by reference.

Selection of these and other common vector and regulatory elements are conventional and many such sequences are available. See, e.g., Sambrook et al, and references cited therein at, for example, pages 3.18-3.26 and 16.17-16.27 and Ausubel et al., Current Protocols in Molecular Biology, John Wiley & Sons, New York, 1989. Of course, not all vectors and expression control sequences will function equally well to express all of the transgenes as described herein. However, one of skill in the art may make a selection among these, and other, expression control sequences without departing from the scope of this invention.

In another embodiment, a method of generating a recombinant adeno-associated virus is provided. A suitable recombinant adeno-associated virus (AAV) is generated by culturing a host cell which contains a nucleic acid sequence encoding an AAV capsid protein as described herein, or fragment thereof; a functional rep gene; a minigene composed of, at a minimum, AAV inverted terminal repeats (ITRs) and a heterologous nucleic acid sequence encoding a desirable transgene; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

Also provided herein are host cells transfected with an AAV as described herein. Most suitably, such a stable host cell will contain the required component(s) under the control of an inducible promoter. However, the required component(s) may be under the control of a constitutive promoter. Examples of suitable inducible and constitutive promoters are provided herein, in the discussion below of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art. In another embodiment, the host cell comprises a nucleic acid molecule (e.g., a plasmid) as described herein.

The minigene, rep sequences, cap sequences, and helper functions required for producing the rAAV described herein may be delivered to the packaging host cell in the form of any genetic element which transfers the sequences carried thereon. The selected genetic element may be delivered by any suitable method, including those described herein. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, NY. Similarly, methods of generating rAAV virions are well known and the selection of a suitable method is not a limitation on the present invention. See, e.g., K. Fisher et al, 1993 J Virol., 70:520-532 and U.S. Pat. No. 5,478,745, among others. These publications are incorporated by reference herein.

Also provided herein, are plasmids for use in producing the vectors described herein. Such plasmids include a nucleic acid sequence encoding at least one of the vp1, vp2, and vp3 of AAVrh94 (SEQ ID NO: 9), AAVrh95 (SEQ ID NO: 11), AAVrh96 (SEQ ID NO: 13), AAVrh97 (SEQ ID NO: 15), AAVrh98 (SEQ ID NO: 17) or AAVrh99 (SEQ ID NO: 19). In further embodiments, the plasmids include a non-AAV sequence. Cultured host cells containing the plasmids described herein are also provided.

In certain embodiments, the plasmids generated are an AAV cis-plasmid encoding the AAV genome and the gene of interest, an AAV trans-plasmid containing AAV rep and the novel hu68 cap gene, and a helper plasmid. These plasmids may be used in any suitable ratio, e.g., about 1 to about 1 to about 1, based on the total weight of the genetic elements. In other embodiments, the pRepCap to AAV cis-plasmid ratio of about 1:1 by weight of each coding sequence and the pHelper is about 2 times the weight. In other embodiments, the ratio may be about 3 to 1 helper: 10 to 1 pRepCap: 1 to 0.10 rAAV plasmid, by weight. Other suitable ratios may be selected. In certain embodiments, the host cell may be stably transformed with one or more of these elements. For example, the host cell may contain a stable nucleic acid molecule comprising the AAVhu68M191 vp1 coding sequence operably linked to regulatory sequences, a nucleic acid molecule encoding the rep coding sequences and/or one or more nucleic acid molecules encoding helper functions (e.g., adenovirus E1a, or the like). In such embodiments, the various genetic elements may be used in any suitable ratio, e.g., about 1 to about 1 to about 1, based on the total weight of the genetic elements. In certain embodiments, the pRep DNA to Cap DNA to the AAV molecule (e.g., plasmid carrying the vector genome to be packaged) ratio of about 1 to about 1 to about 1 (1:1:1) by weight. In certain embodiments, certain host cells contain some helper elements (e.g., Ad E2a and/or AdE2b) provided in trans and others in cis (e.g., Ad E L1a and/or E1b). The helper sequences may be present in about 2 times the amount of the other genetic elements. Still other ratios may be determined.

The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Clement and Grieger, Mol Ther Methods Clin Dev, 2016: 3: 16002, published online 2016 Mar. 16. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A variety of AAV purification methods are known in the art. See, e.g., WO 2017/160360 entitled “Scalable Purification Method for AAV9”, which is incorporated by reference herein, and describes methods generally useful for Clade F capsids. A two-step affinity chromatography purification followed by anion exchange resin chromatography are used to purify the vector drug product and to remove empty capsids. The crude cell harvest may be subject steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector. An affinity chromatography purification followed anion exchange resin chromatography are used to purify the vector drug product and to remove empty capsids. In one example, for the Affinity Chromatography step, the diafiltered product may be applied to a Capture Select™ Poros-AAV2/9 affinity resin (Life Technologies) that efficiently captures the AAV2/9 serotype. Under these ionic conditions, a significant percentage of residual cellular DNA and proteins flow through the column, while AAV particles are efficiently captured. See, also, WO2021/158915; WO2019/241535; and WO 2021/165537. Alternatively, other purification methods may be selected.

Methods for characterization or quantification of rAAV are available to one of skill in the art. For example, to calculate empty and full particle content, VP3 band volumes for a selected sample (e.g., in examples herein an iodixanol gradient-purified preparation where #of GC=#of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles.

In certain embodiments, the yield of packaged AAV vector genome copies (VG or GC) may be assessed through use of a bioactivity assay for the encoded transgene. For example, after production, culture supernatants may be collected and spun down to remove cell debris. The yields may be measured by a bioactivity assay using equal volume of the supernatant from a test sample as compared to a control (reference standard) to transduce a selected target cell and to evaluate bioactivity of the encoded protein. Other suitable methods for assessing yield may be selected, including, for example, nanoparticle tracking [Povlich, S. F., et al. (2016) Particle Titer Determination and Characterization of rAAV Molecules Using Nanoparticle Tracking Analysis. Molecular Therapy: AAV Vectors II, 24(S1), S122], enzyme linked immunosorbent assay (ELISA) [Grimm, D., et al (1999). Titration of AAV-2 particles via a novel capsid ELISA: packaging of genomes can limit production of recombinant AAV-2. Gene therapy, 6(7), 1322-1330, doi.org/10.1038/sj.gt.3300946]; digital droplet (dd) polymerase chain reaction (PCR) Methods for determining single-stranded and self-complementary AAV vector genome titers by digital droplet (dd) polymerase chain reaction (PCR) have been described. See, e.g., M. Lock et al, Hum Gene Ther Methods. 2014 April; 25(2):115-25. doi: 10.1089/hgtb.2013.131. Epub 2014 Feb. 14]. Another suitable method is qPCR. An optimized-PCR method may be used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay. Yet another method is the quantitative DNA dot blot [Wu, Z., et al, (2008). Optimization of self-complementary AAV vectors for liver-directed expression results in sustained correction of hemophilia B at low vector dose. Molecular therapy: the journal of the American Society of Gene Therapy, 16(2), 280-289. doi.org/10.1038/sj.mt.6300355]. Still other methods may be selected.

Methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used. As used herein, the terms genome copies (GC) and vector genomes (vg) in the context of a dose or dosage (e.g., GC/kg and vg/kg) are meant to be interchangeable.

Methods for determining the ratio among vp1, vp2 and vp3 of capsid protein are also available. See, e.g., Vamseedhar Rayaprolu et al, Comparative Analysis of Adeno-Associated Virus Capsid Stability and Dynamics, J Virol. 2013 December; 87(24): 13150-13160; Buller R M, Rose J A. 1978. Characterization of adenovirus-associated virus-induced polypeptides in KB cells. J. Virol. 25:331-338; and Rose J A, Maizel J V, Inman J K, Shatkin A J. 1971. Structural proteins of adenovirus-associated viruses. J. Virol. 8:766-770.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system (e.g., different runs of a production system using the same genetic elements for production). A variety of production systems, including but not limited to those described herein, may be selected.

### C. Pharmaceutical Compositions and Administration

In one embodiment, the recombinant AAV containing the desired transgene and promoter for use in the target cells as detailed above is optionally assessed for contamination by conventional methods and then formulated into a pharmaceutical composition intended for administration to a subject in need thereof. Such formulation involves the use of a pharmaceutically and/or physiologically acceptable vehicle or carrier, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20. In another embodiment, the pharmaceutically acceptable carrier comprises a surfactant, such as perfluorooctane (Perfluoron liquid). The vector is formulated in a buffer/carrier suitable for infusion in human subjects. The buffer/carrier should include a component that prevents the rAAV from sticking to the infusion tubing but does not interfere with the rAAV binding activity in vivo.

In certain embodiments of the methods described herein, the pharmaceutical composition described above is administered to the subject intramuscularly (IM). In other embodiments, the pharmaceutical composition is administered by intravenously (IV). In other embodiments, the pharmaceutical composition is administered by intracerebroventricular (ICV) injection. In other embodiments, the pharmaceutical composition is administered by intra-cisterna magna (ICM) injection. Other forms of administration that may be useful in the methods described herein include, but are not limited to, direct delivery to a desired organ (e.g., the eye), including subretinal or intravitreal delivery, oral, inhalation, intranasal, intratracheal, intravenous, intramuscular, subcutaneous, intradermal, and other parental routes of administration. Routes of administration may be combined, if desired.

As used herein, the terms “intrathecal delivery” or “intrathecal administration” refer to a route of administration via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna.

As used herein, the terms “intracisternal delivery” or “intracisternal administration” refer to a route of administration directly into the cerebrospinal fluid of the cisterna magna cerebellomedularis, more specifically via a suboccipital puncture or by direct injection into the cisterna magna or via permanently positioned tube.

The composition may be delivered in a volume of from about 0.1 μL to about 10 mL, including all numbers within the range, depending on the size of the area to be treated, the viral titer used, the route of administration, and the desired effect of the method. In one embodiment, the volume is about 50 μL. In another embodiment, the volume is about 70 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL. In another embodiment, the volume is about 1.5 mL. In another embodiment, the volume is about 2 mL. In another embodiment, the volume is about 2.5 mL. In another embodiment, the volume is about 3 mL. In another embodiment, the volume is about 3.5 mL. In another embodiment, the volume is about 4 mL. In another embodiment, the volume is about 5 mL. In another embodiment, the volume is about 5.5 mL. In another embodiment, the volume is about 6 mL. In another embodiment, the volume is about 6.5 mL. In another embodiment, the volume is about 7 mL. In another embodiment, the volume is about 8 mL. In another embodiment, the volume is about 8.5 mL. In another embodiment, the volume is about 9 mL. In another embodiment, the volume is about 9.5 mL. In another embodiment, the volume is about 10 mL.

An effective concentration of a recombinant adeno-associated virus carrying a nucleic acid sequence encoding the desired transgene under the control of the regulatory sequences desirably ranges from about 10⁷ and 10¹⁴ vector genomes per milliliter (vg/mL) (also called genome copies/mL (GC/mL)). In one embodiment, the rAAV vector genomes are measured by real-time PCR. In another embodiment, the rAAV vector genomes are measured by digital PCR. See, Lock et al, Absolute determination of single-stranded and self-complementary adeno-associated viral vector genome titers by droplet digital PCR, Hum Gene Ther Methods. 2014 April; 25(2):115-25. doi: 10.1089/hgtb.2013.131. Epub 2014 Feb. 14, which are incorporated herein by reference. In another embodiment, the rAAV infectious units are measured as described in S. K. McLaughlin et al, 1988 J. Virol., 62:1963, which is incorporated herein by reference.

Preferably, the concentration is from about 1.5×10⁹ vg/mL to about 1.5×10¹³ vg/mL, and more preferably from about 1.5×10⁹ vg/mL to about 1.5×10¹¹ vg/mL. In one embodiment, the effective concentration is about 1.4×10¹¹ vg/mL. In one embodiment, the effective concentration is about 3.5×10¹⁰ vg/mL. In another embodiment, the effective concentration is about 5.6×10¹¹ vg/mL. In another embodiment, the effective concentration is about 5.3×10¹² vg/mL. In yet another embodiment, the effective concentration is about 1.5×10¹² vg/mL. In another embodiment, the effective concentration is about 1.5×10¹³ vg/mL. All ranges described herein are inclusive of the endpoints.

In one embodiment, the dosage is from about 1.5×10⁹ vg/kg of body weight to about 1.5×10³ vg/kg, and more preferably from about 1.5×10⁹ vg/kg to about 1.5×10¹¹ vg/kg. In one embodiment, the dosage is about 1.4×10¹² vg/kg. In one embodiment, the dosage is about 3.5×10¹⁰ vg/kg. In another embodiment, the dosage is about 5.6×10¹¹ vg/kg. In another embodiment, the dosage is about 5.3×10¹² vg/kg. In yet another embodiment, the dosage is about 1.5×10¹² vg/kg. In another embodiment, the dosage is about 1.5×10¹³ vg/kg. In another embodiment, the dosage is about 3.0×10¹² vg/kg. In another embodiment, the dosage is about 1.0×10¹⁴ vg/kg. All ranges described herein are inclusive of the endpoints.

In one embodiment, the effective dosage (total genome copies delivered) is from about 10⁷ to 10¹³ vector genomes. In one embodiment, the total dosage is about 10⁸ genome copies. In one embodiment, the total dosage is about 10⁹ genome copies. In one embodiment, the total dosage is about 10¹⁰ genome copies. In one embodiment, the total dosage is about 10¹¹ genome copies. In one embodiment, the total dosage is about 10¹² genome copies. In one embodiment, the total dosage is about 10¹³ genome copies. In one embodiment, the total dosage is about 10¹⁴ genome copies. In one embodiment, the total dosage is about 10¹⁵ genome copies.

It is desirable that the lowest effective concentration of virus be utilized in order to reduce the risk of undesirable effects, such as toxicity. Still other dosages and administration volumes in these ranges may be selected by the attending physician, taking into account the physical state of the subject, preferably human, being treated, the age of the subject, the particular disorder and the degree to which the disorder, if progressive, has developed. Intravenous delivery, for example may require doses on the order of 1.5×10¹³ vg/kg.

### D. Methods

In another aspect, a method of transducing a target cell or tissue is provided. In one embodiment, the method includes administering an rAAV as described herein.

In one embodiment, the dosage of an rAAV is about 1×10⁹ GC to about 1×10¹⁵ genome copies (GC) per dose (to treat an average subject of 70 kg in body weight), and preferably 1.0×10¹² GC to 2.0×10¹⁵ GC for a human patient. In another embodiment, the dose is less than about 1×10¹⁴ GC/kg body weight of the subject. In certain embodiments, the dose administered to a patient is at least about 1.0×10⁹ GC/kg, about 1.5×10⁹ GC/kg, about 2.0×10⁹ GC/g, about 2.5×10⁹ GC/kg, about 3.0×10⁹ GC/kg, about 3.5×10⁹ GC/kg, about 4.0×10⁹ GC/kg, about 4.5×10⁹ GC/kg, about 5.0×10⁹ GC/kg, about 5.5×10⁹ GC/kg, about 6.0×10⁹ GC/kg, about 6.5×10⁹ GC/kg, about 7.0×10⁹ GC/kg, about 7.5×10⁹ GC/kg, about 8.0×10⁹ GC/kg, about 8.5×10⁹ GC/kg, about 9.0×10⁹ GC/kg, about 9.5×10⁹ GC/kg, about 1.0×10¹⁰ GC/kg, about 1.5×10¹⁰ GC/kg, about 2.0×10¹⁰ GC/kg, about 2.5×10¹⁰ GC/kg, about 3.0×10¹⁰ GC/kg, about 3.5×10¹⁰ GC/kg, about 4.0×10¹⁰ GC/kg, about 4.5×10¹⁰ GC/kg, about 5.0×10¹⁰ GC/kg, about 5.5×10¹⁰ GC/kg, about 6.0×10¹⁰ GC/kg, about 6.5×10¹⁰ GC/kg, about 7.0×10¹⁰ GC/kg, about 7.5×10¹⁰ GC/kg, about 8.0×10¹⁰ GC/kg, about 8.5×10¹⁰ GC/kg, about 9.0×10¹⁰ GC/kg, about 9.5×10¹⁰ GC/kg, about 1.0×10¹¹ GC/kg, about 1.5×10¹¹ GC/kg, about 2.0×10¹¹ GC/kg, about 2.5×10¹¹ GC/kg, about 3.0×10¹¹ GC/kg, about 3.5×10¹¹ GC/kg, about 4.0×10¹¹ GC/kg, about 4.5×10¹¹ GC/kg, about 5.0×10¹¹ GC/kg, about 5.5×10¹¹ GC/kg, about 6.0×10¹¹ GC/kg, about 6.5×10¹¹ GC/kg, about 7.0×10¹¹ GC/kg, about 7.5×10¹¹ GC/kg, about 8.0×10¹¹ GC/kg, about 8.5×10¹¹ GC/kg, about 9.0×10¹¹ GC/kg, about 9.5×10¹¹ GC/kg, about 1.0×10¹² GC/kg, about 1.5×10¹² GC/kg, about 2.0×10¹² GC/kg, about 2.5×10¹² GC/kg, about 3.0×10¹² GC/kg, about 3.5×10¹² GC/kg, about 4.0×10¹² GC/kg, about 4.5×10¹² GC/kg, about 5.0×10¹² GC/kg, about 5.5×10¹² GC/kg, about 6.0×10¹² GC/kg, about 6.5×10¹² GC/kg, about 7.0×10¹² GC/kg, about 7.5×10¹² GC/kg, about 8.0×10¹² GC/kg, about 8.5×10¹² GC/kg, about 9.0×10¹² GC/kg, about 9.5×10¹² GC/kg, about 1.0×10¹³ GC/kg, about 1.5×10¹³ GC/kg, about 2.0×10¹³ GC/kg, about 2.5×10¹³ GC/kg, about 3.0×10¹³ GC/kg, about 3.5×10¹³ GC/kg, about 4.0×10¹³ GC/kg, about 4.5×10¹³ GC/kg, about 5.0×10¹³ GC/kg, about 5.5×10¹³ GC/kg, about 6.0×10¹³ GC/kg, about 6.5×10¹³ GC/kg, about 7.0×10¹³ GC/kg, about 7.5×10¹³ GC/kg, about 8.0×10¹³ GC/kg, about 8.5×10¹³ GC/kg, about 9.0×10¹³ GC/kg, about 9.5×10¹³ GC/kg, or about 1.0×10¹⁴ GC/kg body weight or the subject.

In one embodiment, the method further comprises administering an immunosuppressive co-therapy to the subject. Such immunosuppressive co-therapy may be started prior to delivery of an rAAV or a composition as disclosed, e.g., if undesirably high neutralizing antibody levels to the AAV capsid are detected. In certain embodiments, co-therapy may also be started prior to delivery of the rAAV as a precautionary measure. In certain embodiments, immunosuppressive co-therapy is started following delivery of the rAAV. e.g., if an undesirable immune response is observed following treatment.

Immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include prednelisone, a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor-(CD25-) or CD3-directed antibodies, anti-IL-2 antibodies, ciclosporin, tacrolimus, sirolimus, IFN-0, IFN-γ, an opioid, or TNF-α (tumor necrosis factor-alpha) binding agent. In certain embodiments, the immunosuppressive therapy may be started 0, 1, 2, 7, or more days prior to the rAAV administration, or 0, 1, 2, 3, 7, or more days post the rAAV administration. Such therapy may involve a single drug (e.g., prednelisone) or co-administration of two or more drugs, the (e.g., prednisolone, micophenolate mofetil (MMF) and/or sirolimus (i.e., rapamycin)) on the same day. One or more of these drugs may be continued after gene therapy administration, at the same dose or an adjusted dose. Such therapy may be for about 1 week (7 days), two weeks, three weeks, about 60 days, or longer, as needed. In certain embodiments, a tacrolimus-free regimen is selected.

The following examples are illustrative of certain embodiments of the invention and are not a limitation thereon.

### EXAMPLES

Because of their simple genome structure, non-pathogenicity, and broad tissue tropism, researchers have intensively investigated adeno-associated virus (AAV) vectors for applications in therapeutic gene transfer. The majority of AAV sequences discovered thus far have been amplified from mammalian genomic DNA (gDNA) preparations via polymerase chain reaction (PCR)-based methods and then subcloned into plasmids and Sanger-sequenced¹⁻⁵. While adequate for their time, these PCR-based methods do not correct for potential polymerase misincorporation errors or template switching when multiple viruses are present in the same sample.

When studying AAV genome recombination in a host, one must consider the accuracy of the isolated capsid sequences. PCR polymerase-induced recombinants within a mixture of viral sequences can significantly skew recombination and viral fitness analyses⁶. Additionally, for gene therapy vectors constructed with AAV capsid sequences from natural sources, the most accurate representation of the isolated VP1 sequence should be used rather than genes influenced by PCR-mediated errors.

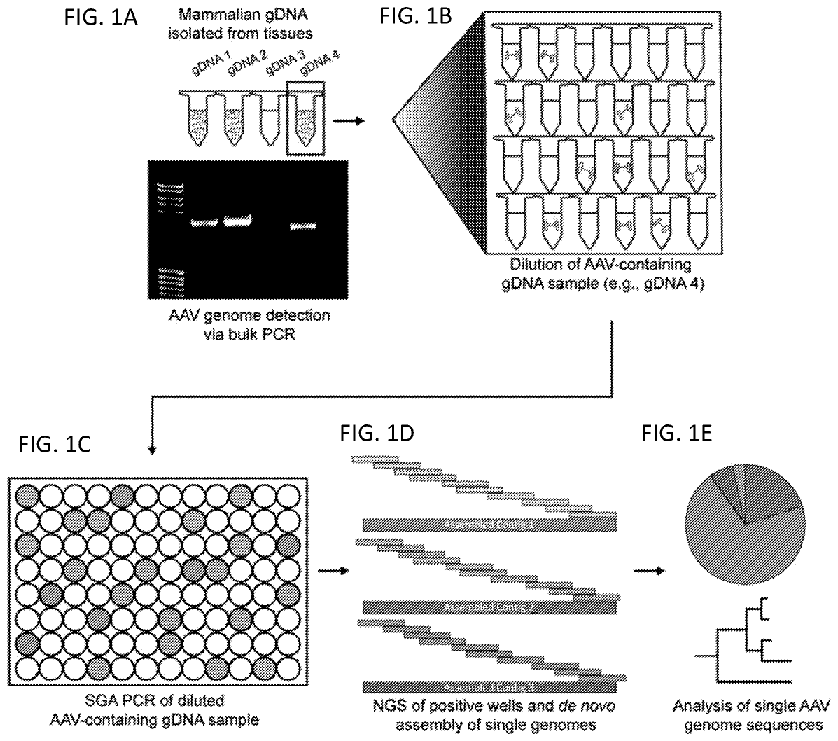

The two major open reading frames (ORFs) in the AAV genome contain the Rep and Cap genes. Whereas Rep encodes four nonstructural proteins related to viral replication functions, Cap encodes three major structural proteins (VP1, VP2, and VP3) that comprise the icosahedral capsid⁷ and two nonstructural proteins^{8,9, 10}. The VP1 sequences of known AAV natural isolates can be phylogenetically grouped into seven major clades: A, B, C, D, E, F, and the Fringe outgroup¹ (Supplemental FIG. 1), whose biological properties differ greatly¹¹⁻¹⁵.

Here, we present AAV single-genome amplification (AAV-SGA), which accurately amplifies and sequences novel AAV populations via a high-fidelity PCR-based method with minimal influences from PCR-mediated amplification errors. We validated AAV-SGA against traditional AAV isolation methods and demonstrated its utility as a robust virus-identification system. Using AAV-SGA, we identified numerous novel AAV genomes from macaque tissue sources. Moreover, we revealed unique AAV phylogenetic properties and analyzed AAV genome recombination events within a primate host. Our results showcase a valuable strategy that can be used to study the complexity and recombination dynamics of AAV genomes in host tissues.

### Materials and Methods

### Primate Tissue DNA Extraction

We collected rhesus macaque tissue samples during necropsy procedures at the University of Pennsylvania Perelman School of Medicine. We extracted gDNA using the QIAamp DNA Mini Kit (QIAGEN, Hilden, Germany).

### AAV-SGA Isolation

To screen primate gDNA for AAV genomes, we used bulk PCR to amplify a 3.1-kb-long AAV sequence with the Q5 Hot Start High-Fidelity DNA Polymerase 2×Master Mix (New England Biolabs [NEB], Ipswich, MA). We utilized the previously described AV1NS forward primer and AV2CAS reverse primer, with the degenerate base Y in AV1NS replaced by a T (AV1NS: 5′-GCTGCGTCAACTGGACCAATGAGAAC-3′ (SEQ ID NO: 39); AV2CAS: 5′-CGCAGAGACCAAAGTTCAACTGAAACGA-3′, SEQ ID NO: 40)4. We used each primer at a final concentration of 0.5 μM, as recommended by the manufacturer. We applied the following thermal cycling conditions: 98° C. for 30 s; 98° C. for 10 s, 59° C. for 10 s, and 72° C. for 93 s for 50 cycles, with a 72° C. extension for 120 s. We then performed AAV-SGA on gDNA samples that screened positive for AAVs based on bulk AAV PCR. AAV-containing gDNA was endpoint-diluted in dilution buffer containing 20 ng/L Ambion™ sheared salmon sperm DNA (Thermo Fisher Scientific, Waltham, MA) and used as a template for one round of PCR with the AV1NS and AV2CAS primers to amplify a 3.1-kb full-length capsid amplicon, as described above. We purified AAV DNA amplicons from positive PCR using Agencourt Ampure XP Beads (Beckman Coulter, Brea, CA). We performed library construction using the NEB Next Ultra™ II DNA Library Prep Kit for Illumina (NEB, Ipswich, MA) and conducted sequencing using the Illumina MiSeq 2×150 or 2×250 paired-end sequencing platform (Illumina, San Diego, CA). The resulting reads were de novo assembled using the Velvet and SPAdes assemblers (cab.spbu.ru/software/spades/and www_ebi_ac_uk/-zerbino/velvet/).

### Conventional Bulk AAV Isolation

We applied bulk PCR, as described above, to amplify a 3,1-kb full-length AAV capsid amplicon from <1,000 ng of nonhuman primate (NHP) gDNA. We performed topoisomerase-based cloning on the PCR products (Invitrogen, Carlsbad, CA) and Sanger-sequencing on individual clones (GENEWIZ, Brisbane, CA).

### Sequence Analysis

We aligned all AAV sequences using AlignX in Vector NTI Advance® 11.5.4 (Invitrogen) and MAFFT alignment in Geneious Prime 2019.2.1 (www_geneious_com). We performed GenBank sequence comparison using the National Center for Biotechnology Information (NCBI) Basic Local Alignment Search Tool server (https://blast_ncbi_nlm_nih_gov/Blast_cgi).

We constructed phylogenetic trees using PhyML 3.0 (http://www_atgc-montpellier_fr/phyml/) or MAFFT version 7 (https://mafft_cbrc_jp/alignment/server/) with the maximum-likelihood and neighbor-joining methods, respectively. Trees were bootstrapped 100 times and formatted using FigTree (http://tree_bio_ed_ac_uk/software/figtree/).

We performed recombination analysis using the Recombination Detection Program (RDP) 4 (web_cbio_uct_ac_za/-darren/rdp_html) under default settings, with the 3.1-kb DNA amplicon sequences as input. A recombination event was deemed likely if at least 6/7 algorithms detected a positive recombination signal. The p-value statistics reported herein are based on the RDP results¹⁶.

### Statistical Analysis

We performed SGA positive amplicon frequency analysis based on the Poisson distribution, as described previously¹⁷. Briefly, the probability of obtaining the observed number (k) of AAV genomes is

$$f ( k ; λ ) = P r ( X = k ) = \frac{{λ}^{k} {e}^{- λ}}{k !} ,$$

where λ is the number of expected AAV genomes per well. For a 30% positive PCR rate, we can estimate λ as:

$$λ : Pr ( X = 0 ) = \frac{{λ}^{0} {e}^{- λ}}{0 !} = {e}^{- λ} = \frac{96 - 29}{96} ⇒ \hat{λ} = - ln \frac{67}{96} = 0.36 .$$

The probability that each positive well contains only one AAV genome is:

$$P r ( X = 1 \vert X \geq 1 ) = \frac{Pr ( X = 1 )}{P r ( X \geq 1 )} = \frac{P r ( X = 1 )}{1 - P r ( X = 0 )} = \frac{\frac{{\hat{λ}}^{1} {e}^{- \hat{λ}}}{1 !}}{1 - \frac{{\hat{λ}}^{0} {e}^{- \hat{λ}}}{0 !}} = \frac{0.25}{1 - 0.7} = 83 \% ,$$

meaning that a positive PCR well contains only one amplifiable AAV genome >80% of the time.

Using a binomial distribution, we derived power calculations determining the proportion of missed viral variants in each AAV-SGA experiment. The probability of k successes among n sequenced amplicons is

$$P r ( X = k ) = ( \begin{matrix}n \\ k\end{matrix} ) {{p}^{k} ( 1 - p )}^{n - k} ,$$

where p is the probability of success. If the fraction of an infrequent virus population is f, the probability of this variant not being sampled among n times is

$$P r ( X = 0 ) = ( \begin{matrix}n \\ 0\end{matrix} ) {{p}^{0} ( 1 - p )}^{n - 0} = {( 1 - f )}^{n} .$$

If the probability equals 0.05, then

$$f = 1 - 0.0 {5}^{\frac{1}{n}} .$$

We used pairwise, two-sample t-tests to compare AAV population distributions in gDNA samples. Percent nucleotide identities for each contig versus consensus sequence were calculated using Geneious Prime. We compared the percent identity means to the consensus for each gDNA sample using t-tests.

See, tables above for the sequences and the sequence listing, incorporated by reference.

### Results

### AAV-SGA Precisely Isolates AAV Capsid Sequences

SGA has been shown to accurately amplify individual virus sequences from mixed samples¹⁷. Based on our knowledge of AAV diversity in animal hosts, we hypothesized that AAV mixtures exist in mammalian tissue samples. Thus, we adapted the original SGA technique to precisely isolate AAV genome populations from mammalian genetic material (FIG. 1). We extracted gDNA from multiple mammalian tissue samples, providing a template for bulk PCR to detect AAV DNA. Following bulk PCR, the resulting 3.1-kb amplicons spanned from the distal third of the Rep gene to the end of the Cap (VP1) gene¹⁸. We amplified AAV genomes from endpoint-diluted gDNA, purified the 3.1-kb amplicons from each positive reaction, and sequenced them using next-generation sequencing (NGS). Because the amplicons arose from a single AAV genome, we could easily assemble them de novo to recover accurate contigs that were uninfluenced by other species in the mixture; therefore, we did not need to deconvolute the amplicons.

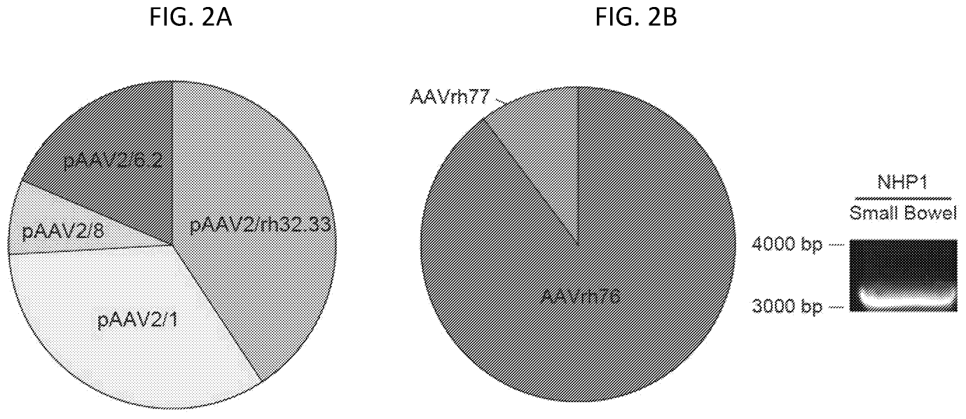

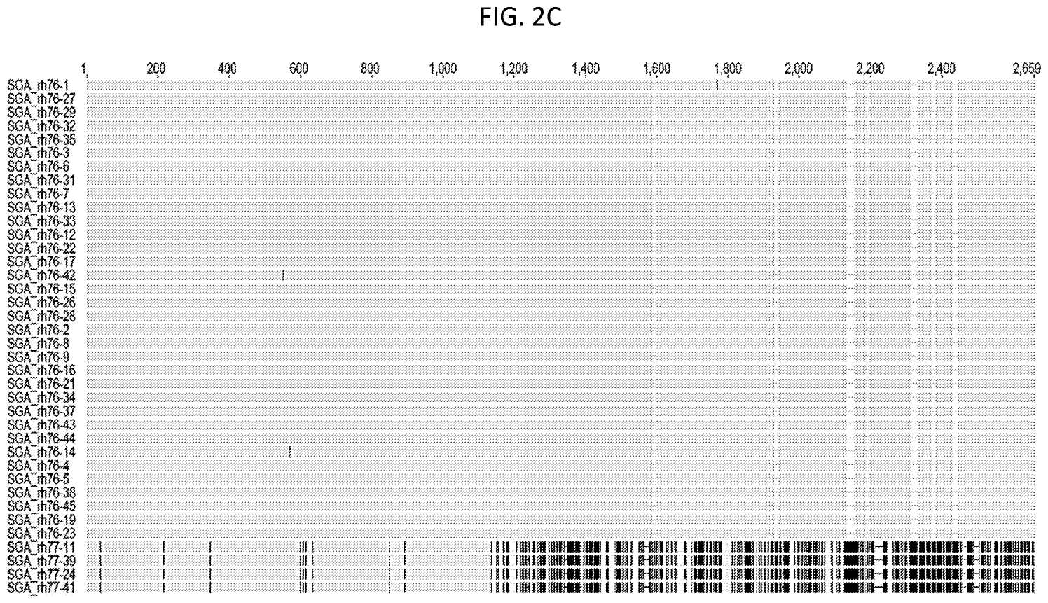

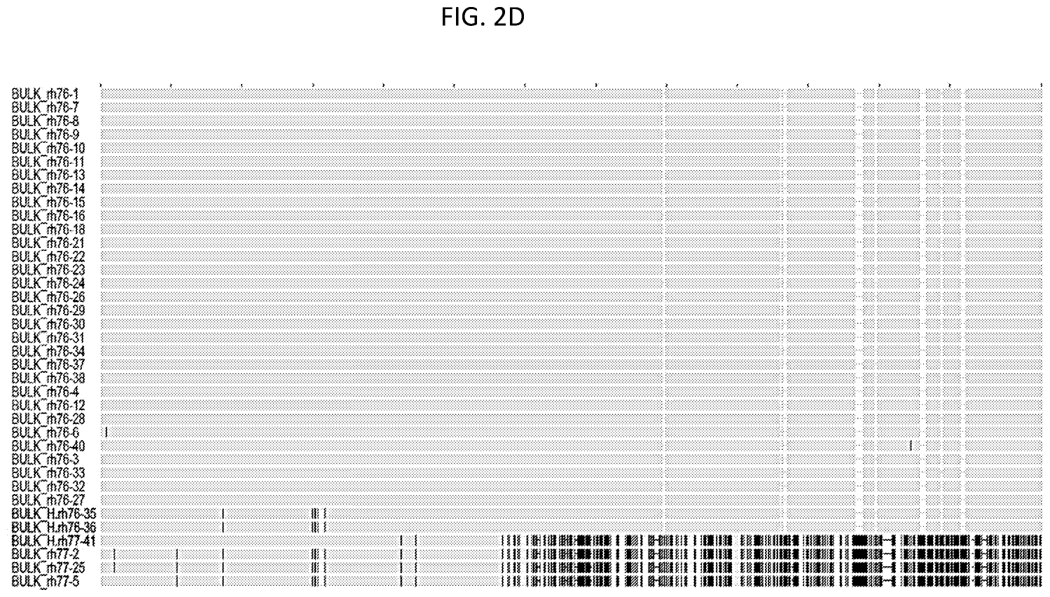

To validate the AAV-SGA method, we utilized a DNA template mixture of four AAV trans plasmids containing the AAV2 Rep ORF and four Cap ORFs: pAAV2/1, pAAV2/6.2, pAAV2/8, pAAV2/rh32.33. We linearized the plasmids with a restriction endonuclease to recapitulate the linear genetic structure of wild-type AAV genomes. Equimolar quantities of each plasmid were mixed and subjected to AAV-SGA. A de novo assembly of 27 amplified single-plasmid sequences demonstrated an accurate recovery of sequences from all four input plasmids (FIG. 2A). All 27 recovered sequences shared 100% identity with the input plasmid sequences, with no evidence of PCR-mediated recombination.

Next, we compared AAV-SGA with the traditional AAV isolation method, which includes bulk AAV PCR of undiluted gDNA, subeloning of the PCR product, and Sanger-sequencing of the resulting plasmids. For both methods, we used the same 3.1-kb amplicon-producing AAV PCR primers and AAV-positive gDNA from a rhesus macaque (NHP1) small bowel tissue sample as the PCR template. AAV-SGA recovered 38 contigs, encompassing two AAV populations: AAVrh76 (90%) and AAVrh77 (10%; FIGS. 2B, 2C). Power calculations determined that any unsampled rare variant could account for <7.6% of the total AAV population. Sequences from the traditional bulk PCR method were then Sanger-sequenced: 31 and 3 contigs had the same sequence as AAVrh76 and AAVrh77, respectively, and 3 contigs were hybrids of AAVrh76 and AAVrh77 (H.rh76-35, H.rh76-36, H.rh77-41; FIG. 2D). H.rh76-41 had the same terminal Rep sequence as AAVrh76, but its VP1 sequence was identical to that of AAVrh77. H.rh76-35 and H.rh76-36 were more similar to AAVrh76 but with sequence signatures of AAVrh77 in their Rep regions. Three bulk contigs were not represented in any sequences isolated by AAV-SGA from the same gDNA sample.

Because the traditional bulk AAV isolation method does not employ limiting dilutions, PCR polymerase template switching, and artificial recombination may occur between multiple genomes (AAVrh76 and AAVrh77). Furthermore, all contigs assembled from the AAV-SGA pipeline resulted from NGS reads, with a coverage depth of >1,000 across the entire amplicon. In contrast, the traditional method utilized Sanger-sequencing, with a coverage depth of 2 across cloned amplicons. Thus, we employed AAV-SGA in all further virus-isolation experiments.

### Rhesus Macaque Tissues Contain Novel AAV Natural Isolates

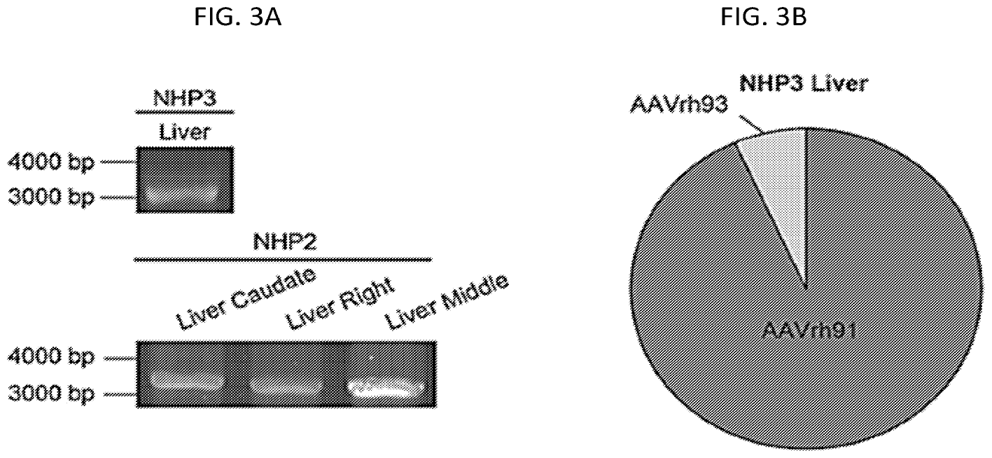

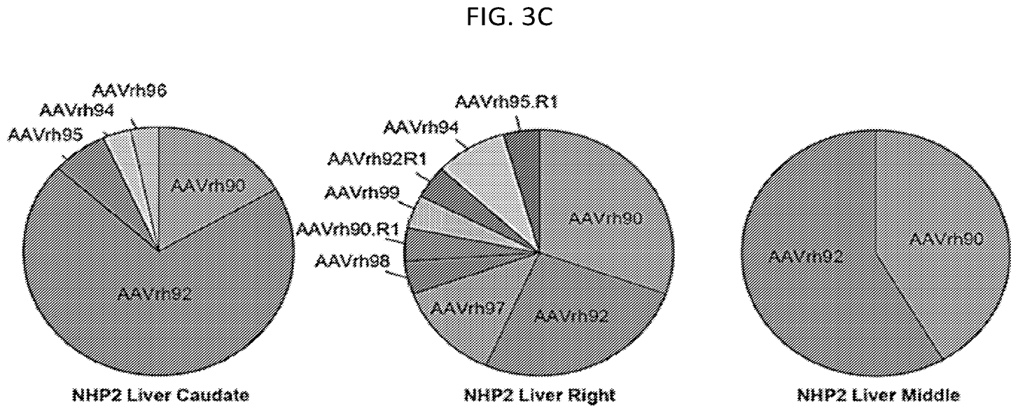

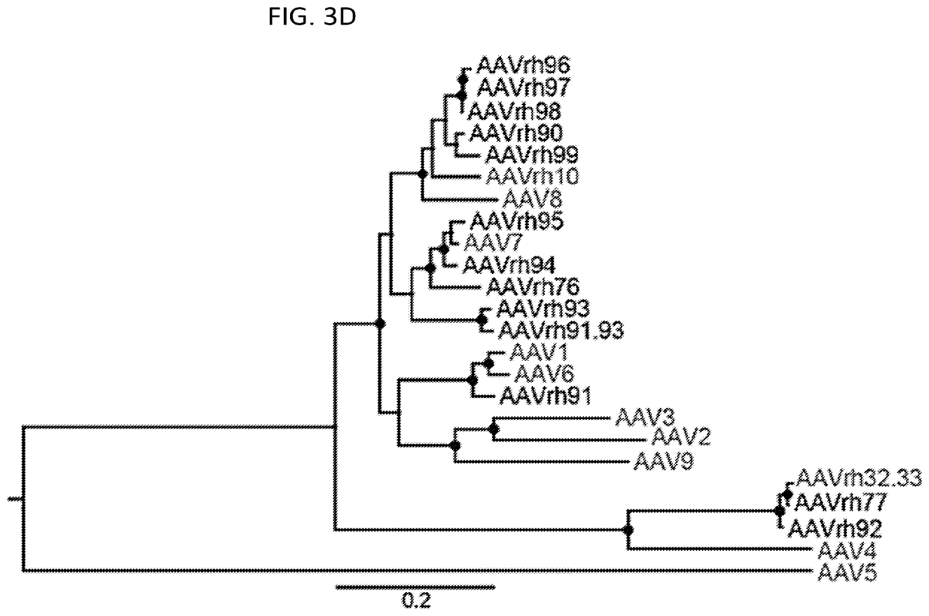

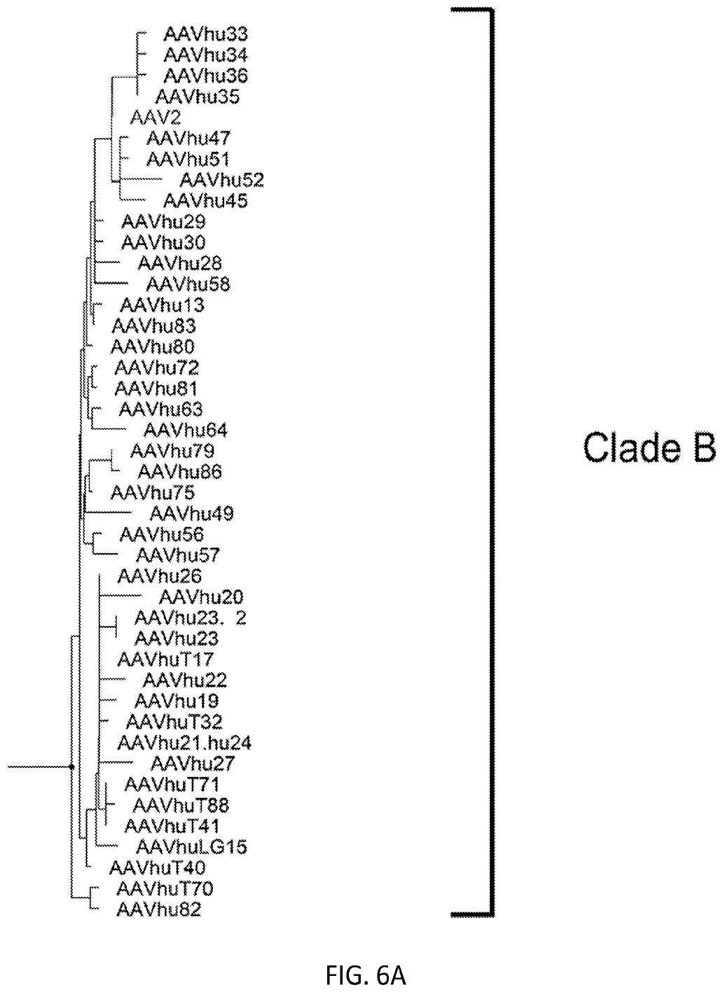

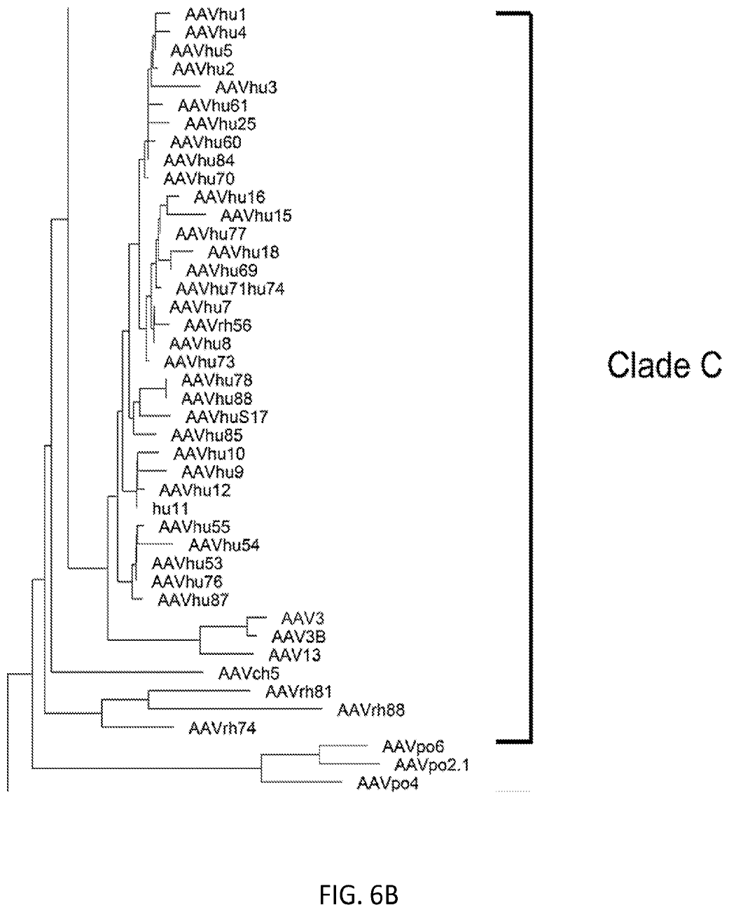

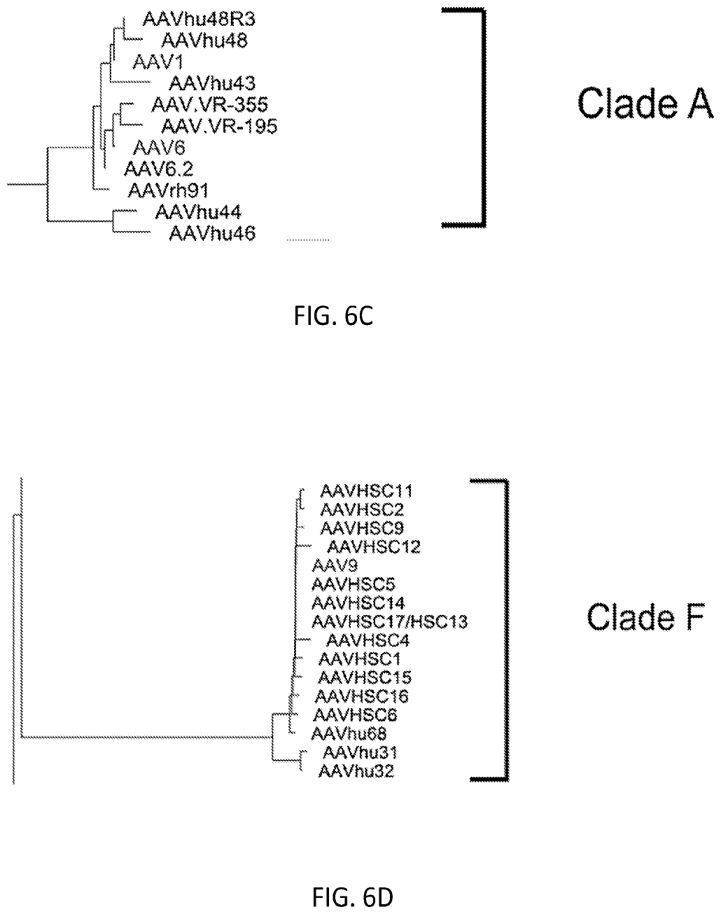

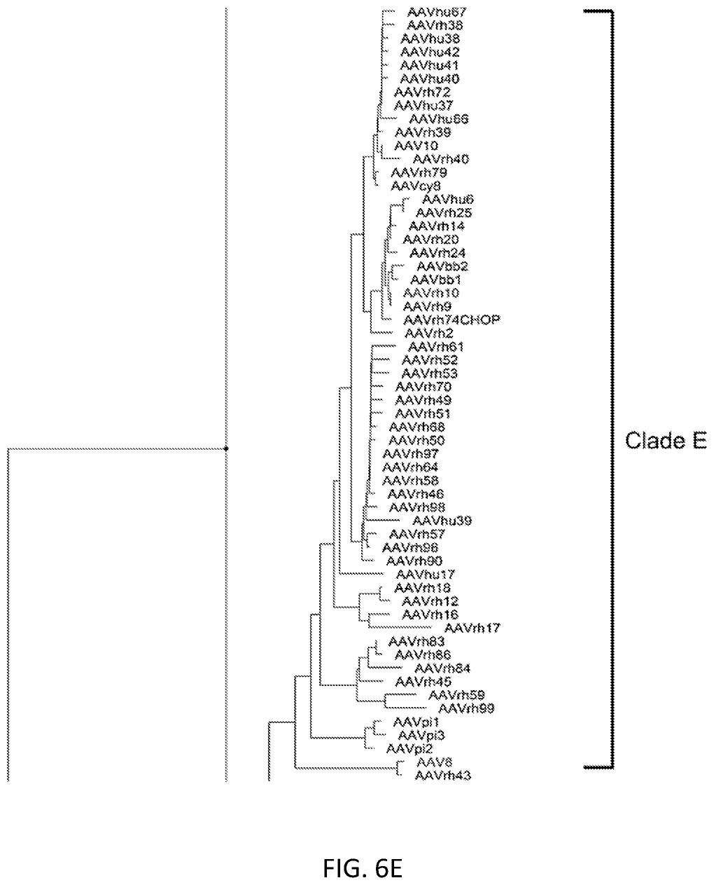

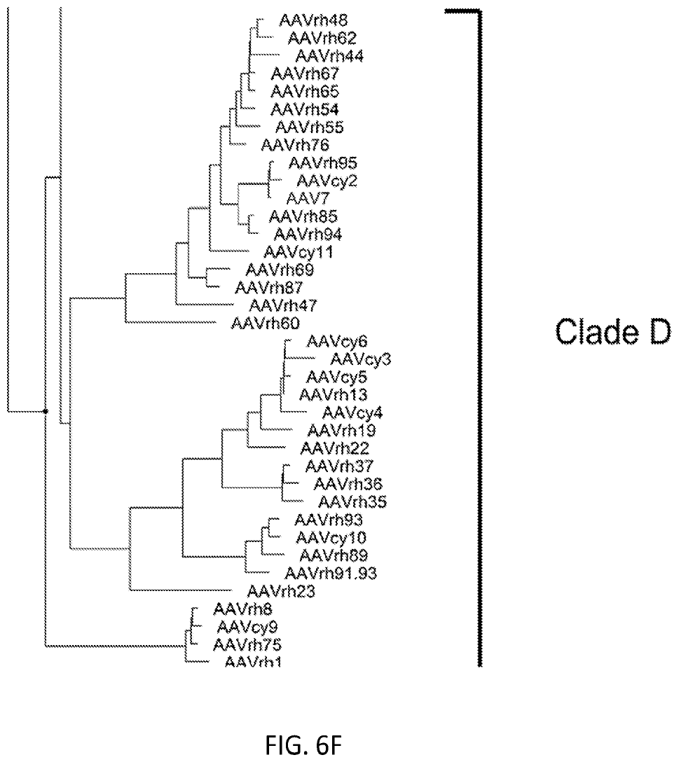

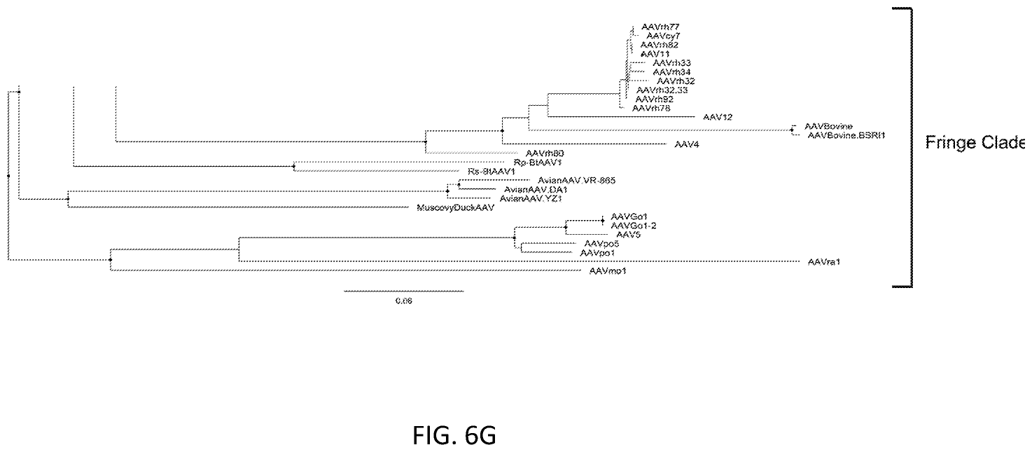

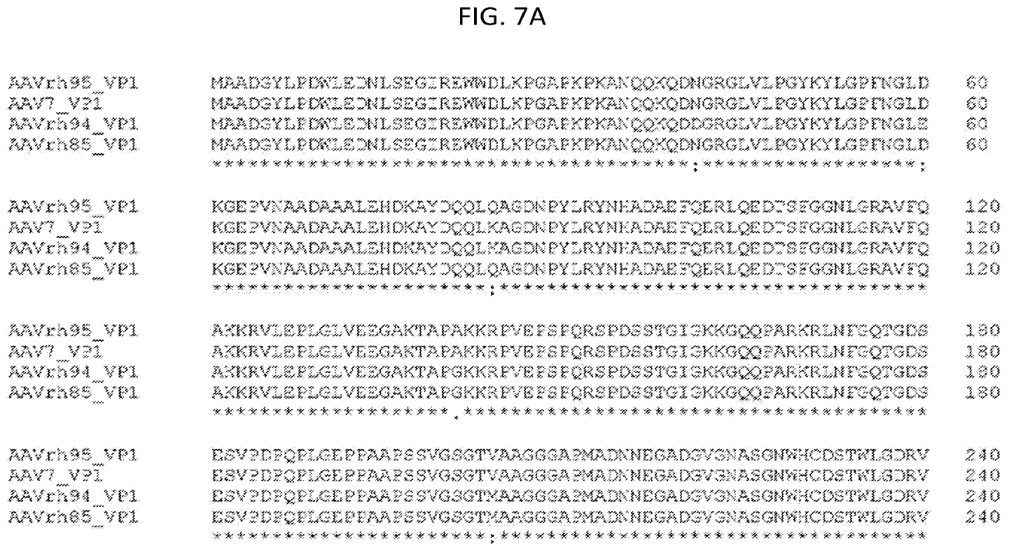

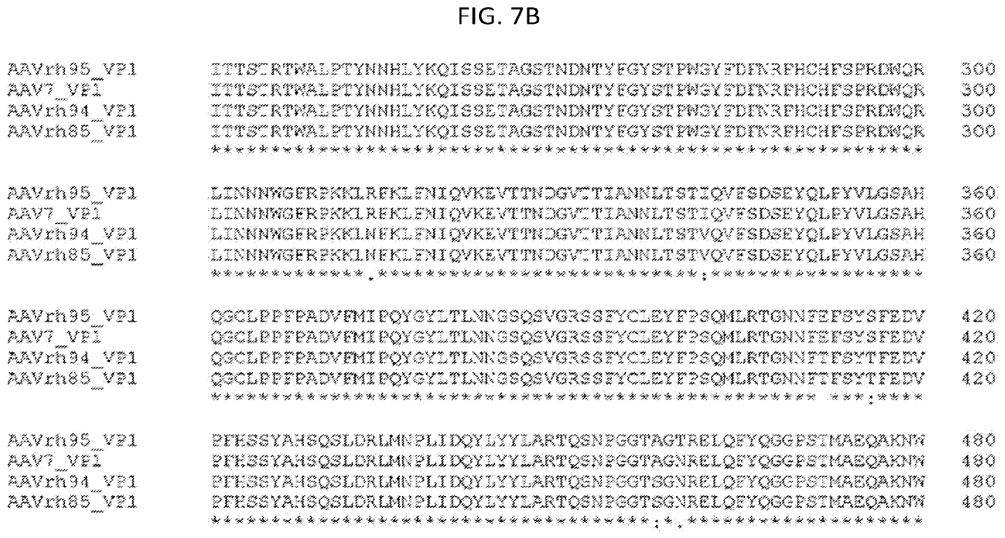

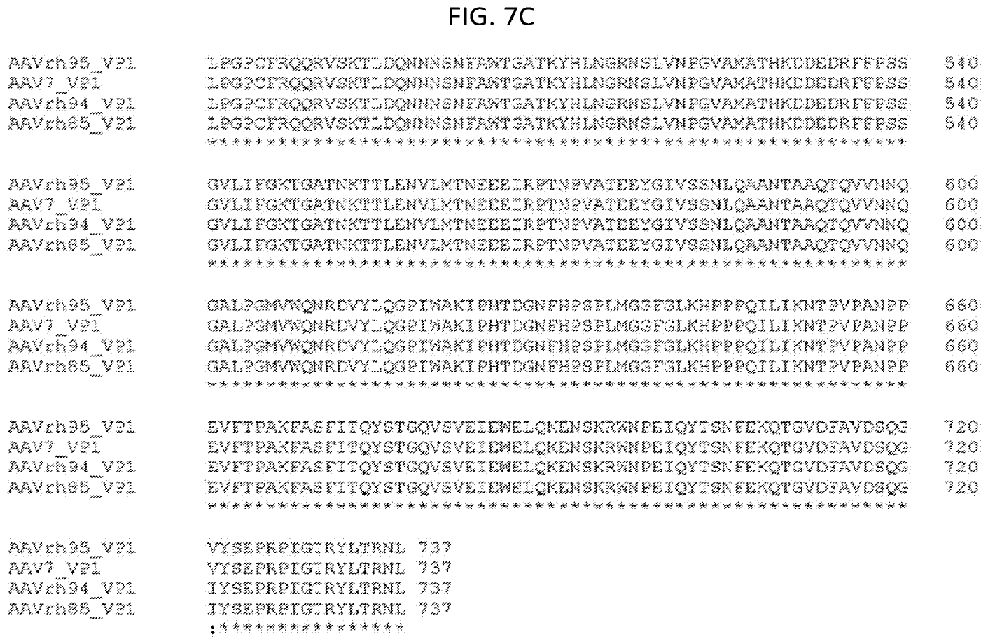

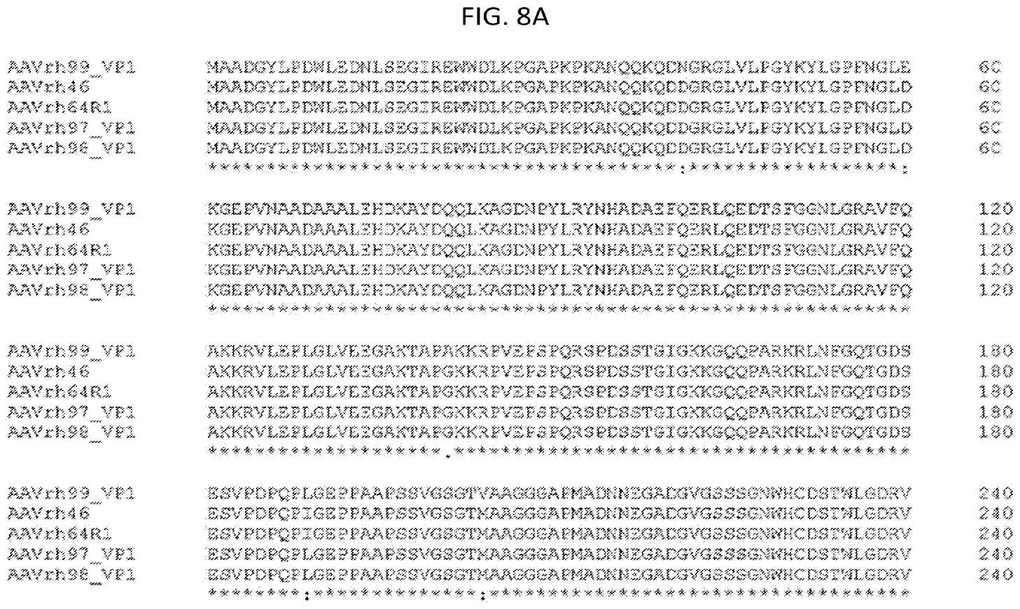

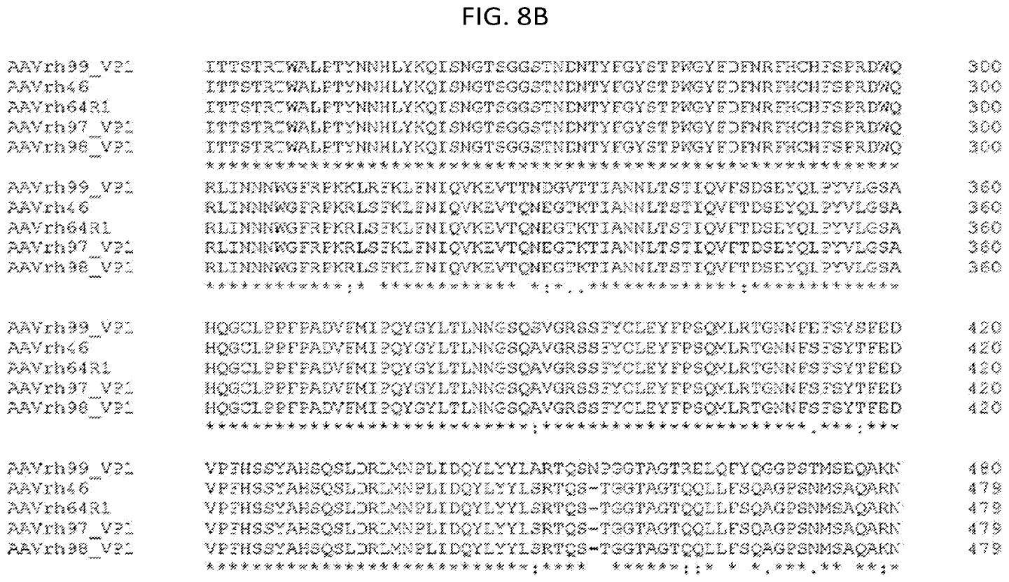

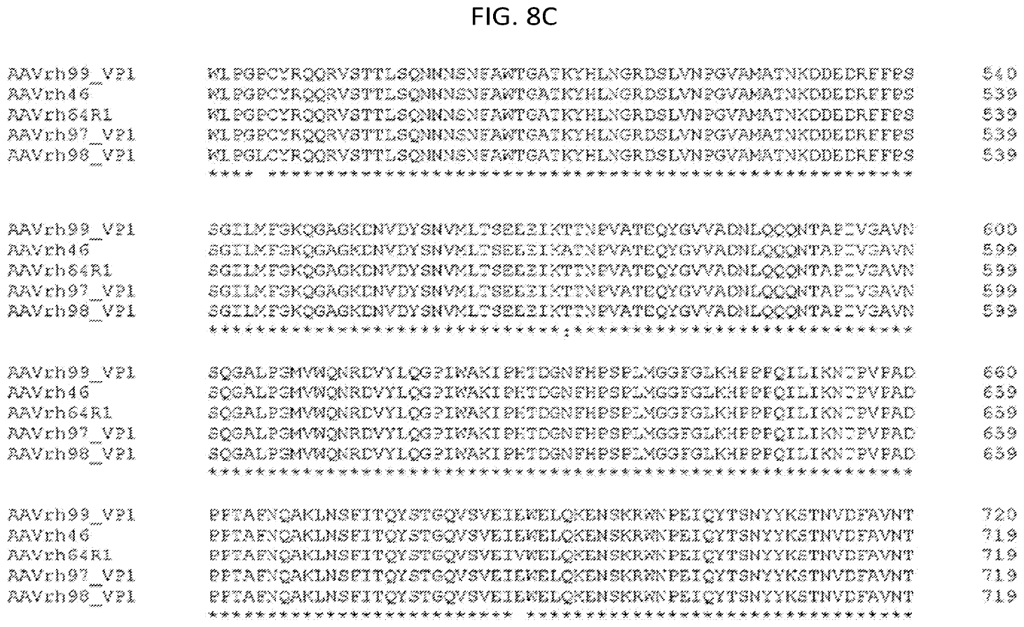

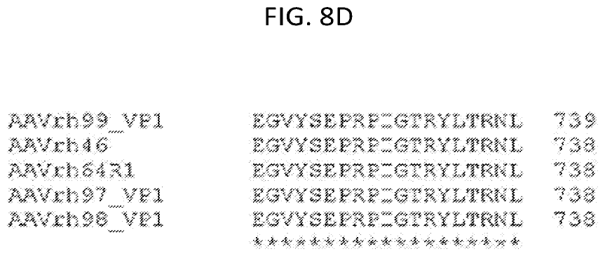

To evaluate AAV diversity in primate tissues, we performed AAV-SGA to isolate novel AAV genome populations from two rhesus macaques. We performed bulk AAV PCR to determine whether liver samples from NHP2 and NHP3 were positive for full-length AAV genomes (FIG. 3A). AAV-SGA of these samples identified two AAV populations: AAVrh91 (clade A) and AAVrh93 (clade D) (FIG. 6). We amplified 11 AAV contigs from the sample from NHP3, with 91% and 9% of sequences originating from AAVrh91 and AAVrh93, respectively. However, power calculations indicated that missed AAV variants could account for <24% of the total virus population, resulting in a potential loss of information.

To test the reproducibility of this result and to uncover potential missed variants, we repeated AAV-SGA on the same liver sample from NHP3 and amplified 30 new amplicons. We recovered similar proportions of AAVrh91 and AAVrh93 (93% and 7%, respectively) (FIG. 3B). With this sample size of 30 amplicons, missed variants would comprise <9.5% of the total virus population. The difference between the proportions of the two AAV populations recovered from the two AAV-SGA trials was non-significant (p=0.345, pairwise t-test), demonstrating the reproducibility of this technique. Thus, SGA provides a snapshot of the AAV species distributed in a source of genetic material.

Three liver lobe samples from NHP2 yielded numerous AAV populations (FIG. 3C). From the right liver lobe, we obtained 23 amplicons, containing nine novel AAV genomes spanning three clades: clade D: AAVrh94, AAVrh95.R1; clade E: AAVrh90, AAVrh90.R1, AAVrh97, AAVrh98, AAVrh99; Fringe clade: AAVrh92, AAVrh92.R1. We recovered 24 AAV amplicons from the middle liver lobe, represented by AAVrh90 and AAVrh92. From the caudate lobe, we obtained five AAVs, spanning 30 amplicons: AAVrh90, AAVrh92, AAVrh94, AAVrh95, and AAVrh96. Power calculations determined that missed variants would comprise 12.2% (right), 11.7% (middle), and 9.5% (caudate) of the total virus population.

AAVrh92 was dominant in the caudate and middle lobes, accounting for 68% and 58%, respectively, whereas AAVrh90 and AAVrh92 accounted for 30% and 26%, respectively, in the right lobe. We compared the percent identities of each isolated sequence within each lobe to the consensus sequence, as constructed from recovered sequences for all three lobes. The population of AAV sequences recovered from the right lobe differed significantly from those for the middle and caudate lobes (Table 1), illustrating regional AAV heterogeneity in the NHP2 liver sample.

TABLE 1
Nucleotide diversity of AAV populations
isolated from the liver of NHP2.

| | Mean percent | | |
|---|---|---|---|
| | nucleotide identity | | |
| Lobe | to consensus | Lobe comparison | p-value |
| Caudate | 92.20 | Right vs. middle | 0.0386 |
| Right | 82.34 | Right vs. caudate | 0.004 |
| Middle | 89.82 | Middle vs. caudate | 0.472 |

p values < 0.05 in bold.

AAVrh90 and AAVrh90.R1 each contained the same VP1 gene sequence in their recovered amplicons but different terminal Rep sequences; this was also the case for AAVrh95 and AAVrh95.R1. We constructed phylogenetic trees from all novel AAV sequences isolated from this study to showcase the diversity of viruses in primate tissues (FIG. 3D; FIG. 6).

### AAV Genomes Recombine in Host Tissues

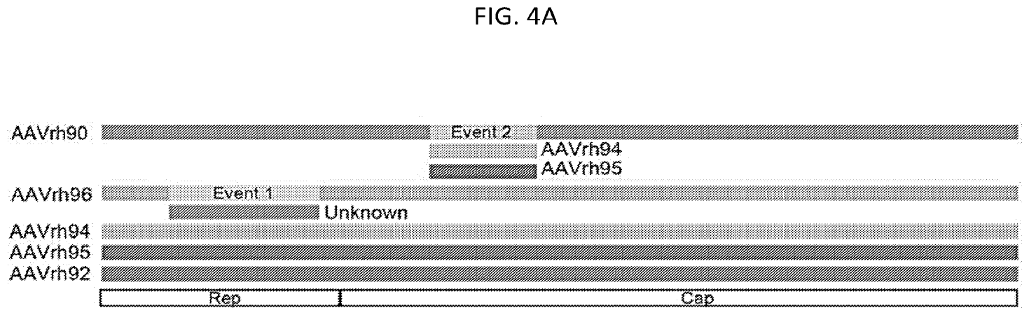

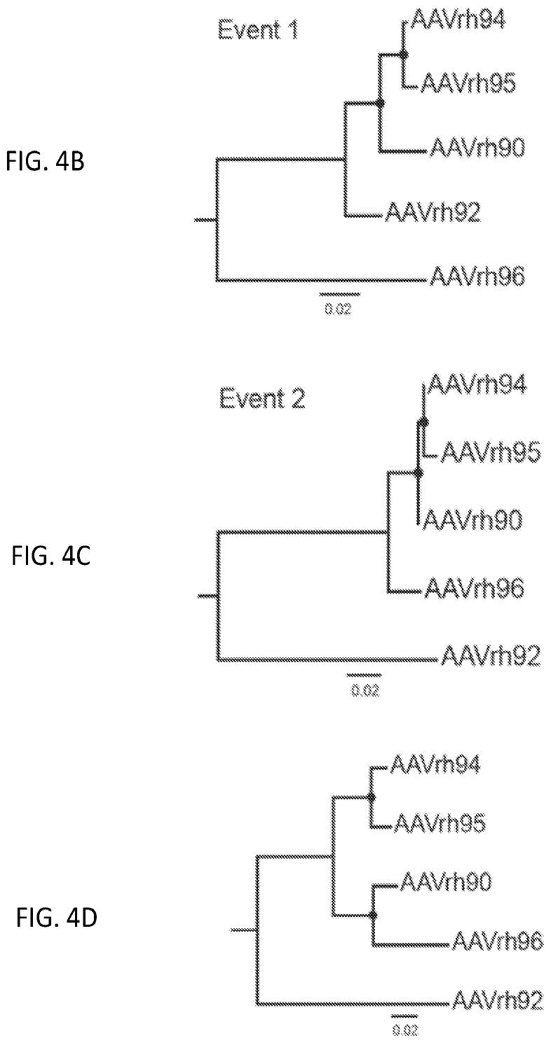

AAV genomes are known to recombine in the host to produce hybrid viruses^{19, 20}. We hypothesized that genome recombination within the NHP2 liver sample contributed to the increased AAV diversity observed. Because we utilized AAV-SGA, we were confident that these isolated sequences did not arise from PCR polymerase template switching. To explore the recombination frequency of AAV genomes isolated from NHP2, we performed sequence analysis using RDP4 (FIGS. 4,5)¹⁶.

We detected two possible recombination events in the AAV sequences from the caudate lobe (FIGS. 4A-C). The first event occurred in the Rep portion of the recombinant AAVrh96 sequence. In this region, AAVrh96 is phylogenetically distinct from all other sequences found in this tissue. Yet, overall, the AAVrh96 sequence is phylogenetically similar to that of AAVrh90 (FIG. 4D), indicating that another AAV or virus not detected by AAV-SGA may have contributed this region of its genome to AAVrh90 to create the recombinant progeny AAVrh96. The second event occurred in the Cap gene of AAVrh90. AAVrh94 and AAVrh95 have high sequence homology to AAVrh90 at this position, suggesting that one of these two viruses may have recombined in this region.

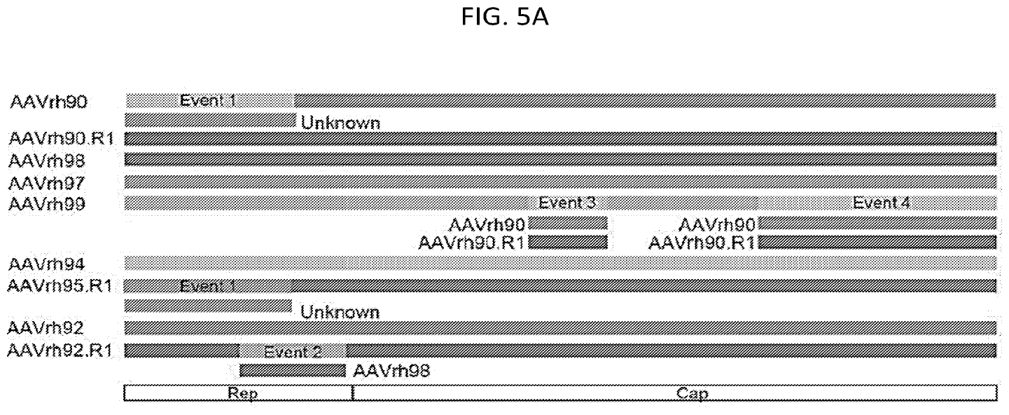

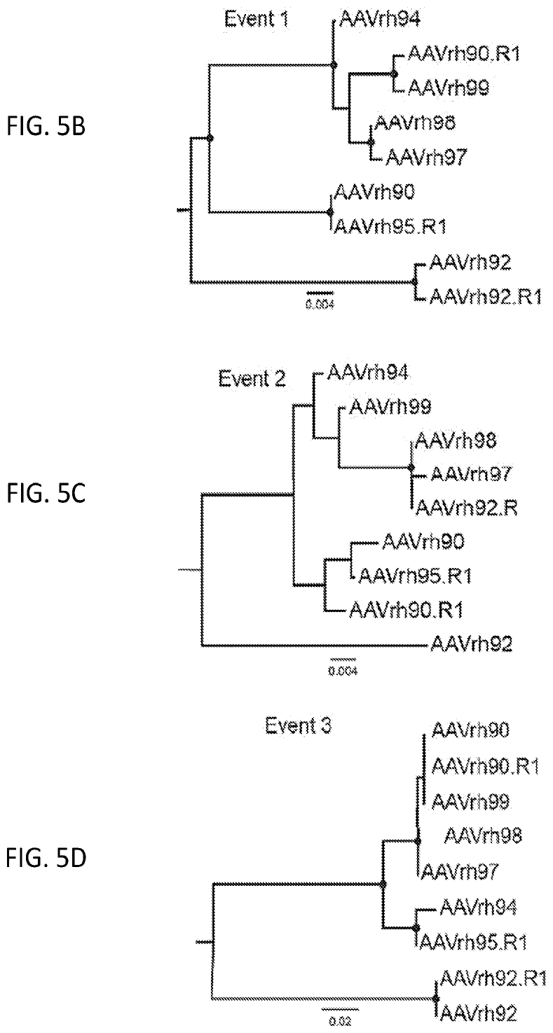

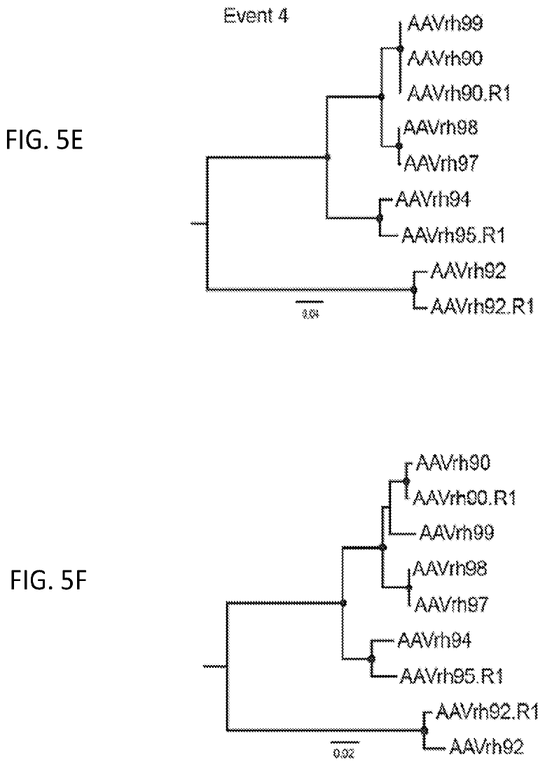

RDP4 detected four recombination events within the sequences recovered from the right liver lobe sample (FIGS. 5A-E). The first event occurred in the distal end of the Rep gene of AAVrh90 and AAVrh95.R1. In this region, AAVrh90 and AAVrh95.R1 have high sequence homology, yet they are not sufficiently similar to other members of the virus group to be considered a contributing recombination parent. Thus, an unknown entity—such as an undetected virus or fragment of the host genome—recombined with AAVrh90 and AAVrh95.R1 in this event. The second recombination event occurred in AAVrh92.R1 at the end of the Rep gene. AAVrh92.R1 and AAVrh98 share high phylogenetic similarity in this region, suggesting that AAVrh98 may have recombined with AAVrh92 to create AAVrh92.R1. We detected two additional events in the Cap region of AAVrh99. AAVrh90 and AAVrh90.R1 have identical VP1 genes; thus, one of these sequences may have contributed to the AAVrh99 recombination events. FIG. 5F shows the phylogenetic relationships of full-length DNA sequences isolated and analyzed from this lobe. Together, these results illustrate how AAV genome recombination can contribute to AAV virome diversity in the liver.

### DISCUSSION

These studies demonstrate the complexity and diversity of AAV genome composition in NHPs. AAV-SGA enabled us to accurately isolate novel AAV genome sequences from several mammalian host tissues. This technique has been adapted to isolate genomes from a wide variety of organisms when conserved primer design strategies are used. Indeed, since its development by Salazar-Gonzalez and colleagues to characterize virus quasispecies in HIV patient samples²⁵, SGA has been used to isolate RNA virus genomes such as HIV and Hepatitis C, as well as parasite genomes like various Plasmodium species^{17, 21-24} To our knowledge, this is the first study to apply SGA to isolate and amplify Parvovirus genomes.

AAV-SGA can distinguish AAV populations in gDNA, in which only one AAV genome is amplified in each SGA PCR step over 80% of the time. This approach circumvents issues caused by possible PCR polymerase template switching when the template DNA sample contains a mixture of species²⁵. When we applied bulk PCR to isolate AAVs from gDNA, we recovered two major AAV populations and various hybrid sequences. In contrast, when we used AAV-SGA, we detected only two discrete populations of viral genomes, with no mosaic sequences. Additionally, using NGS instead of Sanger-sequencing enabled the detection of rare occurrences of heterogeneity in PCR products due to the high sequence coverage of NGS platforms.

We used the Q5 High-Fidelity PCR polymerase, which has a very low error rate. Long-read NGS techniques can also be applied to isolate full-length AAV capsid open-reading frames²⁶. With innovations in sequencing technology and analysis, long-read methods will likely supersede AAV-SGA-based short-read techniques. In the meantime, however, use of AAV-SGA will ensure that any resulting sequence is an accurate representation of the AAV population in a given gDNA sample.

Here, we identified distinct AAV populations in various regions of the rhesus macaque liver and small bowel (FIG. 2) belonging to multiple existing AAV clades (Supplemental FIG. 1). For NHP3, we did not detect significant differences in the distribution or diversity of viral genomes recovered from a single gDNA sample, despite increasing AAV-SGA replicate numbers to increase our analysis power. However, a higher proportion of rare variants can potentially be missed when smaller replicates are used, which may have occurred with the “unknown” sequences identified as contributing factors in two regions of recombination found in viruses extracted from NHP2 liver samples (FIGS. 4, 5).

The vascularization of the liver allows for homogenous blood distribution throughout its lobes²⁸. Thus, we expected virus populations isolated from different liver lobes of the same animal to be homogeneous. However, in NHP2, we isolated different AAV genome distributions from three lobes. Previous work has shown that multiple AAV genomes can exist in the same organ^{1, 18, 19, 29}. However, this is the first study to characterize the microdiversity of AAVs in different regions of an organ from a single animal. These findings suggest that when isolating AAV genomes from the liver, one should assay multiple regions to fully capture the viral diversity.

The factors governing intra-organ heterogeneity of the AAV genome composition in the liver are currently unknown. We can investigate the spatial AAV distribution in mammalian host organs by using AAV-SGA to collect information about viral tropism and transmission. We have also detected recombination events in liver isolates that allowed the introduction of new AAV quasispecies in the host (Table 1, FIG. 5), which could influence the viral fitness landscape under selective pressure^{14, 30-33}, presenting another avenue for future research.

Research has previously characterized homologous recombination in AAVs as a source of viral diversity^{19, 20}. Similarly, we found evidence of recombination events in liver isolates that allowed the introduction of new AAV quasispecies in the host (Table 1, FIG. 5). The introduction of these hybrid sequences could potentially produce capsids with altered hypervariable region composition, which may promote binding to cell surface receptors and neutralizing antibodies^{30, 34-39} and could consequently influence the viral fitness landscape under selective pressure^{14, 30-33}.

In our samples, we primarily detected recombination in the Rep region. The amplicons recovered by AAV-SGA cover only 65% of the complete wild-type AAV genome. The recombination profiles could potentially be slightly altered if larger regions of the Rep gene were analyzed. Four of six possible occurrences of recombination arose near the end of the Rep gene. The higher sequence conservation of the Rep gene in the Dependoparvovirus genus could explain why homologous recombination rates are higher in this region than in the more divergent Cap genes¹. AAV genome recombination studies utilizing the whole viral genome are warranted to further elucidate this aspect.

### CONCLUSION

In summary, a powerful technique for isolating AAV genomes and newly discovered natural capsids are reported. The diversity of AAVs created by genomic recombination-which can be analyzed by AAV-SGA—is a valuable aspect of AAV biology that can be harnessed to develop capsids for use as gene therapy vectors.

### Example 2: Evaluation of Production Yields and Transduction Levels for Recombinant AAV Vectors with Novel Capsids

rAAV vectors are produced and purified using the protocol described by Lock et al. (Human Gene Therapy 21:1259-1271, October 2010). The titers of the purified products are measured by Droplet Digital PCR described by Lock et al. (Human Gene Therapy 25:115-25, April 2014). The three plasmids for the triple-transfection part of the protocol are: adenovirus helper plasmid pAdAF6, a trans plasmid carrying AAV2 rep gene and the capsid gene of an AAVrh94, AAVrh95, AAVrh96, AAVrh97, AAVrh98, or AAV rh99 isolate, and a cis plasmid carrying a transgene cassette flanked by AAV2 5′ and 3′ ITRs. The cis plasmid includes an expression cassette having a TBG promoter and eGFP transgene.

For 12-well plate scale production, the protocol is adapted from the protocol above without the purification step, mainly by reducing the materials used proportionally to cell culture areas. The trans plasmids used here included the AAVrh94, AAVrh95, AAVrh96, AAVrh97, AAVrh98, or AAV rh99 capsid genes. The cis plasmid used includes a CB7 promoter and firefly luciferase gene. After production, culture supernatants are collected and spun down to remove cell debris. The yields are then measured by a bioactivity assay where an equal volume of the supernatants are used to transduce Huh7 and MC57G cells, and luciferase activity is measured with a luminometer (BioTek).

In addition, delivery of transgenes is evaluated in vivo. Mice are injected intravenously with rAAV having an AAVrh94-rh99 capsid and a vector genome containing a human transgene or a reporter gene. On day 28, plasma is collected to measure expression levels.

### REFERENCES

1. Gao G, Vandenberghe L H, Alvira M R et al. Clades of Adeno-Associated Viruses Are Widely Disseminated in Human Tissues. Journal of Virology 2004; 78:6381-6388.
2. Bello a, Tran K, Chand a et al. Isolation and evaluation of novel adeno-associated virus sequences from porcine tissues. Gene therapy 2009; 16:1320-1328.
3. Chen C-L, Jensen R L, Schnepp B C et al. Molecular characterization of adeno-associated viruses infecting children. Journal of virology 2005; 79:14781-14792.
4. Gao G-P, Alvira M R, Wang L et al. Novel adeno-associated viruses from rhesus monkeys as vectors for human gene therapy. Proceedings of the National Academy of Sciences of the United States of America 2002; 99:11854-11859.
5. Mori S, Wang L, Takeuchi T et al. Two novel adeno-associated viruses from cynomolgus monkey: pseudotyping characterization of capsid protein. Virology 2004; 330:375-383.
6. Wang Q, Nambiar K, Wilson J M. Isolating Natural Adeno-Associated Viruses from Primate Tissues with a High-Fidelity Polymerase. Hum Gene Ther 2021; 32:1439-1449.
7. Adeno-Associated Virus Biology, Methods and Protocols. 2011.
8. Sonntag F, Schmidt K, Kleinschmidt J A. A viral assembly factor promotes AAV2 capsid formation in the nucleolus. Proc Natl Acad Sci USA 2010; 107:10220-10225.
9. Elmore Z C, Patrick Havlik L, Oh D K et al. The membrane associated accessory protein is an adeno-associated viral egress factor. Nat Commun 2021; 12:6239.
10. Ogden P J, Kelsic E D, Sinai S et al. Comprehensive AAV capsid fitness landscape reveals a viral gene and enables machine-guided design. Science 2019; 366:1139-1143.
11. Asokan A, Schaffer D V, Samulski R J. The AAV vector toolkit: poised at the clinical crossroads. Molecular therapy: the journal of the American Society of Gene Therapy 2012; 20:699-708.
12. Armstrong D K, Cunningham S, Davies J C et al. Gene therapy in cystic fibrosis. Arch Dis Child 2014; 161:465-468.
13. Ding W, Zhang L, Yan Z et al. Intracellular trafficking of adeno-associated viral vectors. Gene therapy 2005; 12:873-880.
14. Pillay S, Carette J E. Host determinants of adeno-associated viral vector entry. Current Opinion in Virology 2017; 24:124-131.
15. Dudek A M, Pillay S, Puschnik A S et al. An alternate route for adeno-associated virus entry independent of AAVR. Journal of Virology 2018:JVI.02213-02217.
16. Martin D P, Lemey P, Lott M et al. RDP3: a flexible and fast computer program for analyzing recombination. Bioinformatics 2010:2462-2463.
17. Salazar-Gonzalez J F, Bailes E, Pham K T et al. Deciphering human immunodeficiency virus type 1 transmission and early envelope diversification by single-genome amplification and sequencing. Journal of virology 2008; 82:3952-3970.
18. Gao G, Vandenberghe L H, Wilson J M. New recombinant serotypes of AAV vectors. Current gene therapy 2005; 5:285-297.
19. Gao G, Alvira M R, Somanathan S et al. Adeno-associated viruses undergo substantial evolution in primates during natural infections. Proceedings of the National Academy of Sciences of the United States of America 2003; 100:6081-6086.
20. Takeuchi Y, Myers R, Danos 0. Recombination and population mosaic of a multifunctional viral gene, adeno-associated virus cap. PLoS ONE 2008; 3:1-7.
21. Loy D E, Plenderleith L J, Sundararaman S A et al. Evolutionary history of human Plasmodium vivax revealed by genome-wide analyses of related ape parasites. Proceedings of the National Academy of Sciences of the United States of America 2018; 115:E8450-E8459.
22. Hedegaard D L, Tully D C, Rowe I A et al. High resolution sequencing of hepatitis C virus reveals limited intra-hepatic compartmentalization in end-stage liver disease. Journal of Hepatology 2017; 66:28-38.
23. Keele B F, Giorgi E E, Salazar-Gonzalez J F et al. Identification and characterization of transmitted and early founder virus envelopes in primary HIV-1 infection. Proceedings of the National Academy of Sciences of the United States of America 2008; 105:7552-7557.
24. Palmer S, Kearney M, Maldarelli F et al. Multiple, linked human immunodeficiency virus type 1 drug resistance mutations in treatment-experienced patients are missed by standard genotype analysis. Journal of Clinical Microbiology 2005; 43:406-413.
25. Odelberg S J, Weiss R B, Hata A et al. Template-switching during DNA synthesis by Thermus aquaticus DNA polymerase I. Nucleic Acids Research 1995; 23:2049-2057.
26. Hsu H L, Brown A, Loveland A B et al. Structural characterization of a novel human adeno-associated virus capsid with neurotropic properties. Nat Commun 2020; 11:3279.
27. Amarasinghe S L, Su S, Dong X et al. Opportunities and challenges in long-read sequencing data analysis. Genome Biol 2020; 21:30.
28. Brunt E M, Gouw A S, Hubscher S G et al. Pathology of the liver sinusoids. Histopathology 2014; 64:907-920.
29. Mori S, Takeuchi T, Enomoto Y et al. Tissue distribution of cynomolgus adeno-associated viruses AAV10, AAV11, and AAVcy.7 in naturally infected monkeys. Archives of Virology 2008; 153:375-380.
30. Giles A R, Govindasamy L, Somanathan S et al. Mapping an adeno-associated virus 9-specific neutralizing epitope to develop next-generation gene delivery vectors. Journal of virology 2018:JVI.01011-01018.
31. Calcedo R, Wilson J M. Humoral Immune Response to AAV. Frontiers in immunology 2013; 4:341.
32. Henn M R, Boutwell C L, Charlebois P et al. Whole genome deep sequencing of HIV-1 reveals the impact of early minor variants upon immune recognition during acute infection. PLoS pathogens 2012; 8:e1002529.
33. Lee A J, Das S R, Wang W et al. Diversifying Selection Analysis Predicts Antigenic Evolution of 2009 Pandemic HIN1 Influenza A Virus in Humans. Journal of virology 2015; 89:5427-5440.
34. Bell C L, Vandenberghe L H, Bell P et al. The AAV9 receptor and its modification to improve in vivo lung gene transfer in mice. The Journal of clinical investigation 2011; 121:2427-2435.
35. Asokan A, Hamra J B, Govindasamy L et al. Adeno-associated virus type 2 contains an integrin alpha5beta1 binding domain essential for viral cell entry. Journal of virology 2006; 80:8961-8969.
36. Bell C L, Gurda B L, Van Vliet K et al. Identification of the galactose binding domain of the adeno-associated virus serotype 9 capsid. J Virol 2012; 86:7326-7333.
37. Bennett A D, Wong K, Lewis J et al. AAV6 K531 serves a dual function in selective receptor and antibody ADK6 recognition. Virology 2018; 518:369-376.
38. Gurda B L, Raupp C, Popa-Wagner R et al. Mapping a neutralizing epitope onto the capsid of adeno-associated virus serotype 8. Journal of virology 2012; 86:7739-7751.
39. Huang L-Y, Patel A, Ng R et al. Characterization of the Adeno-Associated Virus 1 and 6 Sialic Acid Binding Site. Journal of virology 2016; 90:5219-5230.

All patents, patent publications, and other publications listed in this specification are incorporated herein by reference. While the invention has been described with reference to a particularly preferred embodiment, it will be appreciated that modifications can be made without departing from the spirit of the invention. Such modifications are intended to fall within the scope of the appended claims.

1. A recombinant adeno-associated virus (rAAV) comprising a capsid and a vector genome comprising an expression cassette comprising a nucleic acid sequence encoding a gene product operably linked to expression control sequences, and an AAV 3′ ITR, wherein the rAAV capsid comprises:
(a) (i) an AAVrh94 capsid produced by expressing a nucleic acid sequence encoding the AAVrh94 VP1 of SEQ ID NO: 10, (ii) an AAVrh94 capsid produced by expressing SEQ ID NO: 9 or a sequence at least 99% identical thereto encoding SEQ ID NO: 10; or (iii) at least AAV rh94 VP1 and VP3 proteins which are 95% to 100% deamidated in at least position N57, N263, N384, and/or N514 based on the residue position numbers of SEQ ID NO: 10, and optionally deamidated in other positions;
(b) (i) an AAVrh95 capsid produced by expressing a nucleic acid sequence encoding the AAVrh95 VP1 of SEQ ID NO: 12, (ii) an AAVrh95 capsid produced by expressing SEQ ID NO: 11 or a sequence at least 99% identical thereto encoding SEQ ID NO: 12; or (iii) at least AAV rh95 VP1 and VP3 proteins which are 95% to 100% deamidated in at least position N57, N384, and/or N515 based on the residue position numbers of SEQ ID NO: 12, and optionally deamidated in other positions;
(c) (i) an AAVrh96 capsid produced by expressing SEQ ID NO: 13 or a sequence at least 98% identical thereto;
(d) (i) an AAVrh97 capsid produced by expressing a nucleic acid sequence encoding the AAVrh97 VP1 of SEQ ID NO: 16, (ii) an AAVrh97 capsid produced by expressing SEQ ID NO: 15 or a sequence at least 99% identical thereto encoding SEQ ID NO: 16; or (iii) at least AAV rh97 VP1 and VP3 proteins which are 95% to 100% deamidated in at least position N57, N263, N384, and/or N514 based on the residue position numbers of SEQ ID NO: 16, and optionally deamidated in other positions;
(e) (i) an AAVrh98 capsid produced by expressing a nucleic acid sequence encoding the AAVrh98 VP1 of SEQ ID NO: 18, (ii) an AAVrh98 capsid produced by expressing SEQ ID NO: 17 or a sequence at least 99% identical thereto encoding SEQ ID NO: 18; or (iii) at least AAV rh98 VP1 and VP3 proteins which are 95% to 100% deamidated in at least position N57, N263, N384, and/or N514 based on the residue position of SEQ ID NO: 18, and optionally deamidated in other positions; or
(f) (i) an AAVrh99 capsid produced by expressing a nucleic acid sequence encoding the AAVrh99 VP1 of SEQ ID NO: 20, (ii) an AAVrh99 capsid produced by expressing SEQ ID NO: 19 or a sequence at least 99% identical thereto encoding SEQ ID NO: 20; or (iii) at least AAV rh99 VP1 and VP3 proteins which are 95% to 100% deamidated in at least position N57, N263, N384, and/or N515 based on the residue positions of SEQ ID NO: 20, and optionally deamidated in other positions.

2. The rAAV according to claim 1, further comprising:
(a) AAVrh94 VP3 proteins having the amino acid sequence of about residue 204 to about 737 of SEQ ID NO: 10 (SEQ ID NO: 34) which are 95% to 100% deamidated in at least position N57, N263, N384, and/or N514 based on the residue position numbers of SEQ ID NO: 10, and optionally deamidated in other positions;
(b) AAVrh95 VP3 proteins having the amino acid sequence of about residue 212 to about 737 of SEQ ID NO: 12 (SEQ ID NO: 35) which are 95% to 100% deamidated in at least position N57, N384, and/or N515 based on the residue position numbers of SEQ ID NO: 12, and optionally deamidated in other positions;
(c) AAVrh97 VP3 proteins having the amino acid sequence of about residue 204 to about 739 of SEQ ID NO: 16 (SEQ ID NO: 36) which are 95% to 100% deamidated in at least position N57, N263, N384, and/or N514 based on the residue position numbers of SEQ ID NO: 16, and optionally deamidated in other positions;
(d) AAVrh98 VP3 proteins having the amino acid sequence of about residue 204 to about 738 of SEQ ID NO: 18 (SEQ ID NO: 37) which are 95% to 100% deamidated in at least position N57, N263, N384, and/or N514 based on the residue position of SEQ ID NO: 18, and optionally deamidated in other positions; or
(e) AAVrh99 VP3 proteins having the amino acid sequence of about residue 212 to about 738 of SEQ ID NO: 20 (SEQ ID NO: 37) which are 95% to 100% deamidated in at least position N57, N263, N384, and/or N515 based on the residue positions of SEQ ID NO: 20, and optionally deamidated in other positions.

3. The rAAV of claim 1, wherein the gene product is useful in treating a disorder or disease of the liver and wherein the capsid is AAVrh96, AAVrh97, AAVrh98, or AAVrh99.

4. The rAAV of claim 1, wherein the gene product is a gene editing nuclease.

5. The rAAV of claim 1, wherein the expression cassette comprises a constitutive promoter.

6. The rAAV of claim 1, wherein the expression cassette comprises a tissue-specific promoter.

7. The rAAV of claim 1, wherein the gene product is an antibody or immunogen, and the capsid is selected from AAVrh94 or AAVrh95.

8. The rAAV of claim 1, wherein the gene product is a therapeutic nucleic acid.

9. A host cell in culture containing the rAAV of claim 1.

10. A pharmaceutical composition comprising the rAAV of claim 1, and a physiologically compatible carrier, buffer, adjuvant, and/or diluent.

11. A method of delivering a transgene to a cell, said method comprising the step of contacting the cell with the rAAV of claim 1, wherein said rAAV comprises the transgene.

12. A plasmid comprising a AAV vp1 capsid nucleic acid sequence which is:
(i) an AAVrh94 nucleic acid sequence encoding the AAVrh94 VP1 of SEQ ID NO: 10 or an AAVrh94 capsid produced by expressing SEQ ID NO: 9 or a sequence at least 99% identical thereto encoding SEQ ID NO: 10;
(ii) an AAVrh95 nucleic acid sequence encoding the AAVrh95 VP1 of SEQ ID NO: 12 or an AAVrh95 capsid produced by expressing SEQ ID NO: 11 or a sequence at least 99% identical thereto encoding SEQ ID NO: 12;
(iii) an AAVrh96 nucleic acid sequence produced by expressing SEQ ID NO: 13 or a sequence at least 98% identical thereto;
(iv) an AAVrh97 nucleic acid sequence encoding the AAVrh97 VP1 of SEQ ID NO: 16 or an AAVrh97 capsid produced by expressing SEQ ID NO: 15 or a sequence at least 99% identical thereto encoding SEQ ID NO: 16;
(v) an AAVrh98 nucleic acid sequence encoding the AAVrh98 VP1 of SEQ ID NO: 18 or (ii) an AAVrh98 capsid produced by expressing SEQ ID NO: 17 or a sequence at least 99% identical thereto encoding SEQ ID NO: 18; or
(vi) an AAVrh99 capsid nucleic acid sequence encoding the AAVrh99 VP1 of SEQ ID NO: 20 or an AAVrh99 capsid produced by expressing SEQ ID NO: 19 or a sequence at least 99% identical thereto encoding SEQ ID NO: 20.

13. A method of generating a recombinant adeno-associated virus (rAAV) comprising an AAV capsid, the method comprising culturing a host cell containing:
(a) a molecule encoding an AAV vp1, vp2, and/or vp3 capsid protein of:
(i) an AAVrh94 capsid produced by expressing a nucleic acid sequence encoding the AAVrh94 VP1 of SEQ ID NO: 10 or an AAVrh94 capsid produced by expressing SEQ ID NO: 9 or a sequence at least 99% identical thereto encoding SEQ ID NO: 10;
(ii) an AAVrh95 capsid produced by expressing a nucleic acid sequence encoding the AAVrh95 VP1 of SEQ ID NO: 12 or an AAVrh95 capsid produced by expressing SEQ ID NO: 11 or a sequence at least 99% identical thereto encoding SEQ ID NO: 12;
(iii) an AAVrh96 capsid produced by expressing SEQ ID NO: 13 or a sequence at least 98% identical thereto;
(iv) an AAVrh97 capsid produced by expressing a nucleic acid sequence encoding the AAVrh97 VP1 of SEQ ID NO: 16 or (ii) an AAVrh97 capsid produced by expressing SEQ ID NO: 15 or a sequence at least 99% identical thereto encoding SEQ ID NO: 16;
(v) an AAVrh98 capsid produced by expressing a nucleic acid sequence encoding the AAVrh98 VP1 of SEQ ID NO: 18 or (ii) an AAVrh98 capsid produced by expressing SEQ ID NO: 17 or a sequence at least 99% identical thereto encoding SEQ ID NO: 18; or
(vi) an AAVrh99 capsid produced by expressing a nucleic acid sequence encoding the AAVrh99 VP1 of SEQ ID NO: 20 or an AAVrh99 capsid produced by expressing SEQ ID NO: 19 or a sequence at least 99% identical thereto encoding SEQ ID NO: 20,
(b) a functional rep gene;
(c) a vector genome comprising an AAV 5′ inverted terminal repeats (ITR), a transgene operably linked to expression control sequences, and an AAV 3′ ITR; and
(d) sufficient helper functions to permit packaging of the vector genome into the AAV capsid protein.

14. A cultured host cell containing the plasmid according to claim 13.


SEQUENCE LISTING (54 sequences)

SEQ ID NO: 1 - DNA, 2217 bp, Adeno-associated virus
  source 1..2217: mol_type = genomic DNA
  CDS 1..2214: gene = AAVrh90 capsid protein VP1
  CDS 610..2214: gene = AAVrh90 capsid protein VP3
atggctgccgatggttatcttccagattggctcgaggacaacctctctgagggcattcgcgagtggtgggacctgaaacctggagccccaaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgataatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctggaaagaagagaccggtagagccatcaccacagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacaatggctgcaggcggtggcgcaccaatggcagacaataacgaaggtgccgacggagtgggtagttcctcgggaaattggcattgcgattccacatggctgggcgacagagtcatcaccaccagcacccgaacctgggccctgcccacctacaacaaccacctctacaagcaaatctccaacgggacctcgggaggcagcaccaacgacaacacctacttcggctacagcaccccctgggggtattttgactttaacagattccactgccacttctcaccacgtgactggcagcgacttatcaacaacaactggggattccggcccaagagactcagcttcaagctcttcaacatccaggtcaaggaggtcacgcaaaatgaaggcaccaagaccatcgccaataacctcaccagcaccatccaggtgtttacggactcggaataccagctgccatacgtcctcggctctgcccaccagggctgcctgcctccgttcccggcggacgtcttcatgattcctcagtatggctacctgacgctgaacaacggaagtcaggccgtgggccgttcctccttctactgcctggagtactttccctctcagatgctaagaacgggcaacaacttctccttcagctataccttcgaggacgtgcctttccacagcagctacgcgcacagccagagcctggaccggctgatgaatcccctcattgaccagtacctgtactacctgtcgcggacacaatccacaggaggcacagcgggaactcagcagttgctgttttctcaggccgggcctaacaacatgtctgctcaggccaaaaactggctacccggaccttgttatcggcagcaacgtgtttccacgacactgtcgcaaaacaacaacagcaactttgcctggaccggtgccaccaaataccacctgaacggaagagactctctggtaaatccgggtgtcgccatggcaaccaacaaggacgacgaggaccgcttcttcccatccagcggcatcctcatgtttggcaagcagggggctggaaaagacaacgtggactatagcaacgtgatgctaaccagcgaggaagaaatcaagaccactaaccctgtggccacagaacagtatggtgtggtggcggataacctgcagcagcaaaacacagctcctattgtgggggccgtcaacagccaaggagccttacctggcatggtttggcagaaccgggacgtgtacctgcagggtcccatctgggccaagattcctcacacggatggtaactttcacccgtctcctctcatgggcggctttggacttaaacatccgcctcctcagatcctgatcaagaacactcccgttcctgcggatcctccaacggcgttcaaccaggccaagctgaactctttcatcacgcagtacagcaccggacaagtcagcgtggagatcgagtgggagctgcagaaggagaacagcaagcgctggaacccagagattcagtatacctccaactactacaaatctacaaatgtggactttgctgttaatactgagggtgtttactctgagcctcgccccattggcacccgttacctcacccgtaatctgtaa

SEQ ID NO: 2 - protein, 738 aa, Adeno-associated virus
  REGION 204..738: note = AAVrh90 capsid protein VP3
  REGION 1..738: note = AAVrh90 capsid protein VP1
MAADGYLPDWLEDNLSEGIREWWDLKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 3 - DNA, 2211 bp, Adeno-associated virus
  source 1..2211: mol_type = genomic DNA
  CDS 1..2208: gene = AAVrh91 capsid protein VP1
  gene 607..2208: gene = AAVrh91 capsid protein VP3
atggctgccgatggttatcttccagattggctcgaggacaacctctctgagggcattcgcgagtggtgggcgctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaagcagctaagacggctcctggaaagaaacgtccggtagagcagtcgccccaagaaccagactcctcctcgggcattggcaaatcaggccagcagcccgccaaaaagagactcaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtacggctacctaacgctcaacaatggcagccaggccgtaggacgttcatccttttattgcctggaatatttcccatctcaaatgctgagaacgggcaacaactttaccttcagctacacctttgaagatgtgcctttccacagcagttacgcgcacagccagagcctggacaggctaatgaatcctctaatcgaccagtacctgtattacctaaacagaactcagaatcaatccggaagtgcacaaaacaaggacttgctgtttagccgggggtctccagctggcatgtctgttcagcccaaaaactggctacccgggccctgttaccgacagcagcgtgtttctaaaacaaaaacagacaacaacaacagcaactttacctggactggtgcctccaaatacaatctgaacggacgtgaatccatcattaaccctggcaccgctatggcatcccacaaggacgacgaagacaaattttttcccatgagcggtgttatgatttttggcaaagaaaatgcaggagcatcaaacactgcattagacaatgttatgattacagatgaagaggaaattaaagctaccaaccccgtggccaccgagagatttggaactgtggcagtcaatctccaaagcagcaatacagaccctgcaacaggagacgtgcatgtcatgggggctttacctggcatggtgtggcaagacagagacgtgtacctgcagggtcccatttgggccaagattcctcacacggatggacactttcacccgtctcctcttatgggcggctttggacttaagcacccgcctcctcagatcctcatcaaaaacacgcctgttcctgcgaatcctccggcagagttttcggctacaaagtttgcttcattcatcacccagtactccacaggacaagtgagcgtggaaattgaatgggagctgcagaaagaaaacagtaagcgctggaatcctgaagtgcagtacacctccaactacgcgaaatctgccaacgttgatttcactgtggacaacaatggactttatactgagcctcgccccattggcacccgttaccttacccgtcccctttaa

SEQ ID NO: 4 - protein, 736 aa, Adeno-associated virus
  REGION 1..736: note = AAVrh91 capsid protein VP1
  REGION 203..736: note = AAVrh91 capsid protein VP3
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEAAKTAPGKKRPVEQSPQEPDSSSGIGKSGQQPAKKRLNFGQTGDSESVPDPQPLGEPPETPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKENAGASNTALDNVMITDEEEIKATNPVATERFGTVAVNLQSSNTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 5 - DNA, 2199 bp, Adeno-associated virus
  source 1..2199: mol_type = genomic DNA
  CDS 1..2199: gene = AAVrh92 capsid protein VP1
  CDS 589..2199: gene = AAVrh92 capsid protein VP3
atggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgaggcggacgccgcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggtactcgaacctctgggcctggttgaagaaggtgctaagacggctcctggaaagaagagaccgttagagtcaccacaagagccagactcctcctcgggaatcggcaaaaaaggcaaacaaccagccaaaaagagactcaactttgaagaggacactggagccggagacggaccccctgaaggatcagataccagcgccatgtcttcagacattgaaatgcgtgcagcaccgggcggaaatgctgtcgatgcgggacaaggttccgatggagtgggtaatgcctcgggtgattggcattgcgattccacctggtctgagggcaaggtcacaacaacctcgaccagaacctgggtcttgcccacctacaacaaccacctgtacctgcggctcggaacaacatcaaacagcaacacctacaacggattctccaccccctggggatactttgactttaacagattccactgtcacttctcaccacgtgactggcaaagactcatcaacaacaactggggactacgaccaaaagccatgcgcgttaaaatcttcaatatccaagttaaggaggtcacaacgtcgaacggcgagactacggtcgctaataaccttaccagcacggttcagatatttgcggactcgtcgtatgagctcccgtacgtgatggacgctggacaagaggggagcctgcctcctttccccaatgacgtctttatggtgcctcaatatggctactgtggcatcgtgactggcgagaatcagaaccagacggacagaaatgccttctactgcctggagtattttccttcacaaatgttgagaactggcaacaactttgaaatggcttacaactttgagaaggtgccgttccactcaatgtatgctcacagccagagcctggacagactgatgaatcccctcctggaccagtacctgtggcacttacagtcgactacctctggagggactctgaatcaaggcaatgcagcaaccacatttggaaaaatcaggagtggagactttgccttttacagaaagaactggctgcctgggccttgtgttaagcagcagagattctcaaagactgccagtcaaaactacaagattcctgccagcgggggcgacgctctgttaaagtatgacacccactataccttaaacaaccggtggagcaacatcgcgcccggacctccaatggccacagccggaccttcggatggggacttcagtaacgcccagcttatattccctggaccatctgttaccggaaatacaacaacttcagccaacaatctgttgtttacatcagaagaagaaattgctgccaccaacccaagagacacggacatgtttggccagattgctgacaataatcagaatgctacaactgctcccataaccggcaacgtgactgctatgggagtgctgcctggcatggtgtggcaaaacagagacatttactaccaagggccaatttgggccaagatcccacacgcggacggacattttcatccttcaccgctgattggtgggtttggactgaaacacccgcctccccagatattcatcaagaacactcccgtacctgccaatcctgcgacaaccttcactgcagccagagtggactctttcattacacaatacagcaccggccaggtcgctgttcagattgaatgggaaattgaaaaggaacgctccaaacgctggaatcctgaagtgcagtttacttcaaactatgggaaccagtcttctatgttgtgggctcctgatacaactgggaagtatacagagccgcgggttattggctctcgttatttgactaatcatttg

SEQ ID NO: 6 - protein, 733 aa, Adeno-associated virus
  REGION 1..733: note = AAVrh92 capsid protein VP1
  REGION 197..733: note = AAVrh92 capsid protein VP3
MAADGYLPDWLEDNLSEGIREWWDLKPGAPKPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSNSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGGTLNQGNAATTFGKIRSGDFAFYRKNWLPGPCVKQQRFSKTASQNYKIPASGGDALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 7 - DNA, 2187 bp, Adeno-associated virus
  source 1..2187: mol_type = genomic DNA
  CDS 1..2184: gene = AAVrh93 capsid protein VP1
  CDS 619..2184: gene = AAVrh93 capsid protein VP3
atggctgccgatggttatcttccagattggctcgaggacaacctctctgagggcattcgcgagtggtgggcgctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaaaacggctcctggaaagaagagacccgtagactctccagactcctccacgggcatcggcaagaaaggccagcagcccgcgaaaaagaagctcaactttgggcagactggcgactcagagtcagtccccgaccctcaacctctctcagaacctccagcagcgcccactagtgtgggatctggtacagtggctgcaggcagtggcgcaccaatggcagacaataacgaaggtgccgacggagtgggtaatgcctcaggaaattggcattgcgattccacatggctgggcgacagagtcatcaccaccagcacccgaacctgggccctgcccacctacaacaaccacctctacaagcaaatctccagtcagagcggggctaccaacgacaaccacttctttggctacagcaccccctgggggtattttgacttcaacagattccactgccacttctcaccacgtgactggcagcgactcatcaacaacaactggggattccggcccaagaagctgcggttcaagctcttcaacatccaagtcaaagaggttacgacgaacgacggcgtcacaaccatcgctaataaccttaccagcacgattcaggttttctcggactcggagtaccagctgccgtacgtcctcggctctgcgcaccagggctgcctccctccgttcccggcggacgtcttcatgattcctcagtacggctacctgacgctcaacaacggtagtcagtctgtgggtcgttcttccttctactgcctggaatatttcccctctcaaatgctgagaacgggcaacaactttgaattcagttacaccttcgaggatgtgcctttccacagcagctacgcgcatagccagagcctggaccggctgatgaatcctcttatcgaccagtatctgtactacctggcccggacccagagcaccacgggttccaccagagagctgcagtttcatcaggctgggcccaacactatggccgagcaatcaaagaactggttacctggtccttgcttccggcaacaacgcgtttccaaggtgctggatcagaacgccaacagcaactttgcctggactgctgccactaaatatcacttgaatgggcgtaactctctgaccaatccgggagttcccatggcaacacacaaggacgacgaggacaggttctttcccattaacggggtgctggtttttggcaagaccggagccgccaacaaaacaacgctggaaaatgttctgatgacaaacgaagaagaaatcaaaactaccaacccggtggctacagaagaatatggcgtggtttccagcaatttgcaggctgggactacaaacccacagacactgactgttaacaaccagggggccttacctggcatggtgtggcagaacagggacgtgtacctgcaaggtcccatctgggccaaaattcctcacacggacggcaactttcacccgtctcctttgatgggtggatttggacttaaacacccgcctcctcagatcctaattaaaaacacgcctgttcctgccaatcctccggaggtgtttactcctgccaagtttgcttcgttcatcacgcagtacagcaccggtcaggtcagcgtggaaatcgagtgggagctgcagaaagaaaacagcaagcgctggaacccagagattcagtacacttccaattttgataaatctaataatgtggactttgctgttaacaatgaaggtgtttactctgagcctcgccccattggcactcgctacctcacccgtaacctgtaa

SEQ ID NO: 8 - protein, 728 aa, Adeno-associated virus
  REGION 1..728: note = AAVrh93 capsid protein VP1
  REGION 207..728: note = AAVrh93 capsid protein VP3
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVDSPDSSTGIGKKGQQPAKKKLNFGQTGDSESVPDPQPLSEPPAAPTSVGSGTVAAGSGAPMADNNEGADGVGNASGNWHCDSTWLGDRVITTSTRTWALPTYNNHLYKQISSQSGATNDNHFFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYTFEDVPFHSSYAHSQSLDRLMNPLIDQYLYYLARTQSTTGSTRELQFHQAGPNTMAEQSKNWLPGPCFRQQRVSKVLDQNANSNFAWTAATKYHLNGRNSLTNPGVPMATHKDDEDRFFPINGVLVFGKTGAANKTTLENVLMTNEEEIKTTNPVATEEYGVVSSNLQAGTTNPQTLTVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFDKSNNVDFAVNNEGVYSEPRPIGTRYLTRNL

SEQ ID NO: 9 - DNA, 2214 bp, Adeno-associated virus
  source 1..2214: mol_type = genomic DNA
  CDS 1..2214: gene = AAVrh94 capsid protein VP1
  CDS 610..2214: gene = AAVrh94 capsid protein VP3
atggctgccgatggttatcttccagattggctcgaggacaacctctctgagggcattcgcgagtggtgggacctgaaacctggagccccgaaacccaaagccaaccagcaaaagcaggacgacggccggggtctggtgcttcctggctacaagtacctcggacccttcaacggactcgaaaagggggagcccgtcaacgcggcggacgcagcggccctcgagcacgacaaggcctacgaccagcagctcaaagcgggtgacaatccgtacctgcggtataatcacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctggaaagaagagaccggtagagccgtcaccacagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacaatggctgcaggcggtggcgcaccaatggcagacaataacgaaggtgccgacggagtgggtaatgcctcaggaaattggcattgcgattccacatggctgggcgacagagtcatcaccaccagcacccgaacctgggcccttcccacctacaacaaccacctctacaagcaaatctccagtgaaactgcaggtagtaccaacgacaacacctacttcggctacagcaccccctgggggtattttgacttcaacagattccactgccacttctcaccacgtgactggcagcgactcatcaacaacaactggggattccggcccaagaagctcaacttcaagctcttcaacatccaggtcaaggaggtcacgacgaatgacggcgtcacgaccatcgctaataaccttaccagcacggttcaggtattctcggactcggagtaccagctgccgtacgtcctcggctctgcgcaccagggctgtttgcctccgttcccggcggacgtcttcatgattcctcagtacggctacctgactctcaacaatggcagccaatcggtgggacgttcttccttctactgcctggaatacttcccctctcagatgctgagaacgggtaacaacttcaccttcagctacacctttgaggacgtgcctttccacagcagctacgcgcacagccagagcctggaccggctgatgaatcccctcatcgaccagtacctgtactacctggctagaacacagagtaacccaggaggcacatctggcaatcgggaactgcagttttaccagggcggcccttctactatggccgaacaagccaagaactggttacctggaccctgcttccggcaacagagagtgtccaaaacgctggatcaaaacaacaacagcaactttgcttggactggtgccaccaagtatcacctgaacggcagaaactcattggttaatcctggtgtcgccatggcaactcacaaggacgacgaggaccgatttttcccatccagcggagtcctgatttttggaaaaactggagcaaccaacaaaactacattggaaaacgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtatctgcagggtcccatctgggccaagattcctcacacggatggcaactttcacccgtctcctctcatgggcggctttggacttaaacatccgcctcctcagattctcatcaagaacactcccgttcctgctaatcctccggaggtgtttactcctgccaagtttgcttcgtttatcacgcagtacagcaccggacaagtcagcgtggagatcgagtgggagctgcagaaggagaacagcaagcgctggaatccagagattcagtatacctccaactttgagaaacagactggtgtggactttgctgttgacagccagggtatttactctgagcctcgccccattggcacccgttacctcacccgtaatctgtaa

SEQ ID NO: 10 - protein, 737 aa, Adeno-associated virus
  REGION 1..737: note = AAVrh94 capsid protein VP1
  REGION 204..737: note = AAVrh94 capsid protein VP3
MAADGYLPDWLEDNLSEGIREWWDLKPGAPKPKANQQKQDDGRGLVLPGYKYLGPFNGLEKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLNFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLARTQSNPGGTSGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGIYSEPRPIGTRYLTRNL

SEQ ID NO: 11 - DNA, 2214 bp, Adeno-associated virus
  source 1..2214: mol_type = genomic DNA
  CDS 1..2211: gene = AAVrh95 capsid protein VP1
  CDS 634..2214: gene = AAVrh95 capsid protein VP3
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcaccgcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcatcaccaccagcacccgaacctgggccctgcccacctacaacaaccacctctacaagcaaatctccagtgaaactgcaggtagcaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggagtaccagctgccgtacgtcctcggctctgcgcaccagggctgcctgcctccgttcccggcggacgtcttcatgattcctcagtacggctacctgacgctcaacaatggcagtcagtctgtgggacgttcctccttctattgcctggagtacttcccttctcagatgctgagaacgggcaacaacttcgagttcagctacagcttcgaggacgtgcctttccacagcagctacgcgcacagccagagcctggaccggctgatgaaccctctcatcgaccagtacctgtactacctggccagaacacagagtaacccaggaggcacagctggcactcgggaactgcagttttaccagggcgggccttccaccatggctgaacaagccaagaactggttacccggaccttgcttccggcaacaaagagtctccaaaacgctggatcaaaataacaacagcaactttgcttggactggtgccaccaaatatcacctgaacggcagaaactcattggttaatcctggtgtcgccatggcaactcacaaggacgacgaggaccgctttttcccatccagcggagtactgatttttggaaaaactggagcaactaacaaaactacattggaaaacgtgttaatgacaaatgaagaagaaattcgtcctactaatcctgtggccacagaagaatacgggatagtcagcagcaacttacaagcggccaatactgcagcccagacacaagttgtcaacaaccagggagccttacctggcatggtttggcagaaccgggatgtgtacttgcagggtcccatctgggccaagattcctcacacggacggcaactttcacccgtctcctctcatgggcggctttggactgaagcatccgcctcctcagatcctgatcaagaatacacccgtacctgctaatcctccggaggtatttactcctgccaagtttgcttctttcatcacacagtacagcaccggacaagtcagcgtggagatcgagtgggagctgcagaaggagaacagcaagcgctggaacccggagattcagtacacctccaactttgaaaagcagactggtgtggactttgcagttgacagccagggtgtttactctgagcctcgccccattggcactcgttacctcacccgtaacctgtaa

SEQ ID NO: 12 - protein, 737 aa, Adeno-associated virus
  REGION 1..737: note = AAVrh95 capsid protein VP1
  REGION 212..737: note = AAVrh95 capsid protein VP3
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGTRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 13 - DNA, 2266 bp, Adeno-associated virus
  source 1..2266: mol_type = genomic DNA
  CDS 1..2266: gene = AAVrh96 capsid
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgttaacgcggcggacgcagcggccctcgagcacgacaaggcctacgaccagcagctcaaagcgggtgacaatccgtacctgcggtataatcacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggcagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggttacgcagaatgaaggcaccaagaccatcgccaataacctcaccagcaccatccaggtgtttacggactcggaataccagctgccgtacgtcctcggctctgcccaccagggctgcctgcctccgttcccggcggacgtcttcatgattcctcagtacggctacctgactctcaacaatggcagccaggccgtgggacgttcctccttctactgcctggagtacttcccctctcagatgctgagaacgggcaacaacttctccttcagctacaccttcgaagacgtgcctttccacagcagctacgcgcacagccagagcctggaccggctgatgaatcccctcatcgaccaatacctgtactacctgtcaagaacccagtctacgggcggtacagcgggaacgcagcagttgctgttttctcaggccgggcctagcaacatgtcggctcaggccagaaactggctgccaggaccctgctacagacagcagcgagtctccacgacactgtcgcaaaacaacaacagcaactttgcttggaccggtgccaccaaatatcacctgaacggcagagactctctggtgaatccgggcgtagccatggcaaccaacaaggacgacgaggaccgcttcttcccatctagcggcatcctcatgtttggcaagcagggagctggaaaagacaacgtggactatagcaacgtgatgctcaccagcgaggaagagatcaagaccaccaaccccgtggccacagaacagtatggcgtggtggctgataacctgcagcagcaaaacaccgctcctattgtaggggccgtcaacagccagggagccttacctggtatggtctggcagaaccgggacgtatacctgcagggtccaatctgggccaagattcctcacacggatggcaactttcacccgtctcctctcatgggcggctttggacttaaacatccgcctcctcagatcctcatcaagaacactcctgttcctgcggaccctccaacggcgttcaaccaggccaagctgaattctttcatcacgcagtacagcaccggacaagtcagcgtggagattgagtgggagctgcagaaggaaaacagcaagcgctggaacccagagattcagtatacctccaactactacaaatctacaaatgtggactttgctgtcaatacagagggtgtttattctgagcctcgccccattggcactcgttacctcacccgtaatctgtaattgcttgttaatcaataaaccggttgattcgtttcagttgaactttgg

SEQ ID NO: 14 - protein, 739 aa, Adeno-associated virus
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGSHQRQHLLRLQHPLGVFLQIPLPLFTTLAATHQQQLGIPAQETQLQALQHPGQGGYAERHQDHRQPHQHHPGVYGLGIPAAVRPRLCPPGLPASVPGGRLHDSSVRLPDSQQWQPGRGTFLLLLPGVLPLSDAENGQQLLLQLHLRRRAFPQQLRAQPEPGPADESPHRPIPVLPVKNPVYGRYSGNAAVAVFSGRAQHVGSGQKLAARTLLQTAASLHDTVAKQQQQLCLDRCHQISPERQRLSGESGRSHGNQQGRRGPLLPIRHPHVWQAGSWKRQRGLQRDAHQRGRDQDHQPRGHRTVWRGGPAAAKHRSYCRGRQQPGSLTWYGLAEPGRIPAGSNLGQDSSHGWQLSPVSSHGRLWTTSASSDPHQEHSCSCGPSNGVQPGQAEFFHHAVQHRTSQRGDVGAAEGKQQALEPRDSVYLQLLQIYKCGLCCQYRGCLFASPHWHSLPHPSVIACSINRLIRFSTL

SEQ ID NO: 15 - DNA, 2217 bp, Adeno-associated virus
  source 1..2217: mol_type = genomic DNA
  CDS 1..2214: gene = AAVrh97 capsid protein VP1
  CDS 310..2214: gene = AAVrh97 capsid protein VP3
atggctgccgatggttatcttccagattggctcgaggacaacctctctgagggcattcgcgagtggtgggacctgaaacctggagccccgaaacccaaagccaaccagcaaaagcaggacgacggccggggtctggtgcttcctggctacaagtacctcggacccttcaacggactcgacaagggggagcccgttaacgcggcggacgcagcggccctcgagcacgacaaggcctacgaccagcagctcaaagcgggtgacaatccgtacctgcggtataatcacgccgacgccgagtttcaggagcgtctgcaagaagatacgtcatttgggggcaacctcgggcgagcagtcttccaggccaagaagcgggttctcgaacctctcggtttggttgaggaaggcgctaagacggctcctggaaagaagagaccggtagaaccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacaatggctgcaggcggtggcgcaccaatggcagacaataacgaaggtgccgacggagtgggtagttcctcgggaaattggcattgcgattccacatggctgggcgacagagtcatcaccaccagcacccgaacctgggccctgcccacctacaacaaccacctctacaagcaaatctccaacgggacctcgggaggcagcaccaacgacaacacctacttcggctacagcaccccctgggggtattttgactttaacagattccactgccacttctcaccacgtgactggcagcgactcatcaacaacaactggggattccggcccaagagactcagcttcaagctcttcaacatccaggtcaaggaggttacgcagaatgaaggcaccaagaccatcgccaataacctcaccagcaccatccaggtgtttacggactcggaataccagctgccgtacgtcctcggctctgcccaccagggctgcctgcctccgttcccggcggacgtcttcatgattcctcagtacggctacctgactctcaacaatggcagccaggccgtgggacgttcctccttctactgcctggagtacttcccctctcagatgctgagaacgggcaacaacttctccttcagctacaccttcgaagacgtgcctttccacagcagctacgcgcacagccagagcctggaccggctgatgaatcccctcatcgaccaatacctgtactacctgtcaagaacccagtctacgggcggtacagcgggaacgcagcagttgctgttttctcaggccgggcctagcaacatgtcggctcaggccagaaactggctgccaggaccctgctacagacagcagcgagtctccacgacactgtcgcaaaacaacaacagcaactttgcttggaccggtgccaccaaatatcacctgaacggcagagactctctggtgaatccgggcgtagccatggcaaccaacaaggacgacgaggaccgcttcttcccatctagcggcatcctcatgtttggcaagcagggagctggaaaagacaacgtggactatagcaacgtgatgctcaccagcgaggaagagatcaagaccaccaaccccgtggccacagaacagtatggcgtggtggctgataacctgcagcagcaaaacaccgctcctattgtaggggccgtcaacagccagggagccttacctggtatggtctggcagaaccgggacgtatacctgcagggtccaatctgggccaagattcctcacacggatggcaactttcacccgtctcctctcatgggcggctttggacttaaacatccgcctcctcagatcctcatcaagaacactcctgttcctgcggaccctccaacggcgttcaaccaggccaagctgaattctttcatcacgcagtacagcaccggacaagtcagcgtggagattgagtgggagctgcagaaggaaaacagcaagcgctggaacccagagattcagtatacctccaactactacaaatctacaaatgtggactttgctgtcaatacagagggtgtttattctgagcctcgccccattggcactcgttacctcacccgtaatctgtaa

SEQ ID NO: 16 - protein, 738 aa, Adeno-associated virus
  REGION 1..738: note = AAVrh97 capsid protein VP1
  REGION 204..738: note = AAVrh97 capsid protein VP3
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 17 - DNA, 2217 bp, Adeno-associated virus
  source 1..2217: mol_type = genomic DNA
  CDS 1..2214: gene = AAVrh98 capsid protein VP1
  CDS 610..2214: gene = AAVrh98 capsid protein VP3
atggctgccgatggttatcttccagattggctcgaggacaacctctctgagggcattcgcgagtggtgggacctgaaacctggagccccgaaacccaaagccaaccagcaaaagcaggacgacggccggggtctggtgcttcctggctacaagtacctcggacccttcaacggactcgacaagggggagcccgttaacgcggcggacgcagcggccctcgagcacgacaaggcctacgaccagcagctcaaagcgggtgacaatccgtacctgcggtataatcacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctggaaagaagagaccggtagaaccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacaatggctgcaggcggtggcgcaccaatggcagacaataacgaaggtgccgacggagtgggtagttcctcgggaaattggcattgcgattccacatggctgggcgacagagtcatcaccaccagcacccgaacctgggccctgcccacctacaacaaccacctctacaagcaaatctccaacgggacctcgggaggcagcaccaacgacaacacctacttcggctacagcaccccctgggggtattttgactttaacagattccactgccacttctcaccacgtgactggcagcgactcatcaacaacaactggggattccggcccaagagactcagcttcaagctcttcaacatccaggtcaaggaggttacgcagaatgaaggcaccaagaccatcgccaataacctcaccagcaccatccaggtgtttacggactcggaataccagctgccgtacgtcctcggctctgcccaccagggctgcctgcctccgttcccggcggacgtcttcatgattcctcagtacggctacctgactctcaacaatggcagccaggccgtgggacgttcctccttctactgcctggagtacttcccctctcagatgctgagaacgggcaacaacttctccttcagctacaccttcgaagacgtgcctttccacagcagctacgcgcacagccagagcctggaccggctgatgaatcccctcatcgaccaatacctgtactacctgtcaagaacccagtctacgggcggtacagcgggaacgcagcagttgctgttttctcaggccgggcctagcaacatgtctgctcaggccaaaaactggctgccaggactctgctacagacagcagcgagtctccacgacactgtcgcaaaacaacaacagcaactttgcttggaccggtgccaccaaatatcacctgaacggcagagactctctggtgaatccgggcgtagccatggcaaccaacaaggacgacgaggaccgcttcttcccatccagcggcatcctcatgtttggcaagcagggagctggaaaagacaacgtggactatagcaacgtgatgctcaccagcgaggaagagatcaagaccaccaaccccgtggccacagaacagtatggcgtggtggctgataacctgcagcagcaaaacaccgctcctattgtaggggccgtcaacagccagggagccttacctggtatggtctggcagaaccgggacgtatacctgcagggtccaatctgggccaagattcctcacacggatggcaactttcacccgtctcctctcatgggcggctttggacttaaacatccgcctcctcagatcctcatcaagaacactcctgttcctgcggaccctccaacggcgttcaaccaggccaagctgaattctttcatcacgcagtacagcaccggacaagtcagcgtggagattgagtgggagctgcagaaggaaaacagcaagcgctggaacccagagattcagtatacctccaactactacaaatctacaaatgtggactttgctgtcaatacagagggtgtttattctgagcctcgccccattggcactcgttacctcacccgtaatctgtaa

SEQ ID NO: 18 - protein, 738 aa, Adeno-associated virus
  REGION 1..738: note = AAVrh98 capsid protein VP1
  REGION 204..738: note = AAVrh98 capsid protein VP3
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQAKNWLPGLCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 19 - DNA, 2220 bp, Adeno-associated virus
  source 1..2220: mol_type = genomic DNA
  CDS 1..2217: gene = AAVrh99 capsid protein VP1
  CDS 634..2217: gene = AAVrh99 capsid protein VP3
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgaaaagggggagcccgtcaacgcggcggacgcagcggccctcgagcacgacaaggcctacgaccagcagctcaaagcgggtgacaatccgtacctgcggtataatcacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcaccgcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtagttcctcgggaaattggcattgcgattccacatggctgggcgacagagtcatcaccaccagcacccgaacctgggccctgcccacctacaacaaccacctctacaagcaaatctccaacgggacctcgggaggcagcaccaacgacaacacctacttcggctacagcaccccctgggggtattttgactttaacagattccactgccacttctcaccacgtgactggcagcgacttatcaacaacaactggggattccggcccaagaagctgcggttcaagctcttcaacatccaggtcaaggaggtcacgacgaatgacggcgttacgaccatcgctaataaccttaccagcacgattcaggtattctcggactcggagtaccagctgccgtacgtcctcggctctgcgcaccagggctgcctgcctccgttcccggcggacgtcttcatgattcctcagtatggctacctgacgctcaacaacggaagtcagtctgtgggacgttcctccttctattgcctggagtacttcccttctcagatgctgagaacgggcaacaacttcgagttcagctacagcttcgaggacgtgcctttccacagcagctacgcgcacagccagagcctggaccggctgatgaaccctctcatcgaccagtacctgtactacctggccagaacacagagtaacccaggaggcacagctggcactcgggaactgcagttttaccagggcgggccttccaccatgtctgaacaagccaaaaactggctacccggaccttgttatcggcagcaacgtgtttccacgacactgtcgcaaaacaacaacagcaactttgcctggaccggtgccaccaaataccacctgaacggaagagactctctggtaaatccgggtgtcgccatggcaaccaacaaggacgacgaggaccgcttcttcccatccagcggcatcctcatgtttggcaagcagggggctggaaaagacaacgtggactatagcaacgtgatgctaaccagcgaggaagaaatcaagaccactaaccctgtggccacagaacagtatggtgtggtggcggataacctgcagcagcaaaacacagctcctattgtgggggccgtcaacagccaaggagccttacctggcatggtttggcagaaccgggacgtgtacctgcagggtcccatctgggccaagattcctcacacggatggtaactttcacccgtctcctctcatgggcggctttggacttaaacatccgcctcctcagatcctgatcaagaacactcccgttcctgcggatcctccaacggcgttcaaccaggccaagctgaactctttcatcacgcagtacagcaccggacaagtcagcgtggagatcgagtgggagctgcagaaggagaacagcaagcgctggaacccagagattcagtatacctccaactactacaaatctacaaatgtggactttgctgttaatactgagggtgtttactctgagcctcgccccattggcacccgttacctcacccgtaatctgtaa

SEQ ID NO: 20 - protein, 739 aa, Adeno-associated virus
  REGION 1..739: note = AAVrh99 capsid protein VP1
  REGION 212..739: note = AAVrh99 capsid protein VP3
MAADGYLPDWLEDNLSEGIREWWDLKPGAPKPKANQQKQDNGRGLVLPGYKYLGPFNGLEKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGTRELQFYQGGPSTMSEQAKNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 21 - DNA, 1608 bp, Adeno-associated virus
  source 1..1608: mol_type = genomic DNA
  CDS 1..1605: gene = AAVrh90 capsid protein VP3
atggctgcaggcggtggcgcaccaatggcagacaataacgaaggtgccgacggagtgggtagttcctcgggaaattggcattgcgattccacatggctgggcgacagagtcatcaccaccagcacccgaacctgggccctgcccacctacaacaaccacctctacaagcaaatctccaacgggacctcgggaggcagcaccaacgacaacacctacttcggctacagcaccccctgggggtattttgactttaacagattccactgccacttctcaccacgtgactggcagcgacttatcaacaacaactggggattccggcccaagagactcagcttcaagctcttcaacatccaggtcaaggaggtcacgcaaaatgaaggcaccaagaccatcgccaataacctcaccagcaccatccaggtgtttacggactcggaataccagctgccatacgtcctcggctctgcccaccagggctgcctgcctccgttcccggcggacgtcttcatgattcctcagtatggctacctgacgctgaacaacggaagtcaggccgtgggccgttcctccttctactgcctggagtactttccctctcagatgctaagaacgggcaacaacttctccttcagctataccttcgaggacgtgcctttccacagcagctacgcgcacagccagagcctggaccggctgatgaatcccctcattgaccagtacctgtactacctgtcgcggacacaatccacaggaggcacagcgggaactcagcagttgctgttttctcaggccgggcctaacaacatgtctgctcaggccaaaaactggctacccggaccttgttatcggcagcaacgtgtttccacgacactgtcgcaaaacaacaacagcaactttgcctggaccggtgccaccaaataccacctgaacggaagagactctctggtaaatccgggtgtcgccatggcaaccaacaaggacgacgaggaccgcttcttcccatccagcggcatcctcatgtttggcaagcagggggctggaaaagacaacgtggactatagcaacgtgatgctaaccagcgaggaagaaatcaagaccactaaccctgtggccacagaacagtatggtgtggtggcggataacctgcagcagcaaaacacagctcctattgtgggggccgtcaacagccaaggagccttacctggcatggtttggcagaaccgggacgtgtacctgcagggtcccatctgggccaagattcctcacacggatggtaactttcacccgtctcctctcatgggcggctttggacttaaacatccgcctcctcagatcctgatcaagaacactcccgttcctgcggatcctccaacggcgttcaaccaggccaagctgaactctttcatcacgcagtacagcaccggacaagtcagcgtggagatcgagtgggagctgcagaaggagaacagcaagcgctggaacccagagattcagtatacctccaactactacaaatctacaaatgtggactttgctgttaatactgagggtgtttactctgagcctcgccccattggcacccgttacctcacccgtaatctgtaa

SEQ ID NO: 22 - DNA, 1605 bp, Adeno-associated virus
  source 1..1605: mol_type = genomic DNA
  CDS 1..1602: gene = AAVrh91 capsid protein VP3
atggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtacggctacctaacgctcaacaatggcagccaggccgtaggacgttcatccttttattgcctggaatatttcccatctcaaatgctgagaacgggcaacaactttaccttcagctacacctttgaagatgtgcctttccacagcagttacgcgcacagccagagcctggacaggctaatgaatcctctaatcgaccagtacctgtattacctaaacagaactcagaatcaatccggaagtgcacaaaacaaggacttgctgtttagccgggggtctccagctggcatgtctgttcagcccaaaaactggctacccgggccctgttaccgacagcagcgtgtttctaaaacaaaaacagacaacaacaacagcaactttacctggactggtgcctccaaatacaatctgaacggacgtgaatccatcattaaccctggcaccgctatggcatcccacaaggacgacgaagacaaattttttcccatgagcggtgttatgatttttggcaaagaaaatgcaggagcatcaaacactgcattagacaatgttatgattacagatgaagaggaaattaaagctaccaaccccgtggccaccgagagatttggaactgtggcagtcaatctccaaagcagcaatacagaccctgcaacaggagacgtgcatgtcatgggggctttacctggcatggtgtggcaagacagagacgtgtacctgcagggtcccatttgggccaagattcctcacacggatggacactttcacccgtctcctcttatgggcggctttggacttaagcacccgcctcctcagatcctcatcaaaaacacgcctgttcctgcgaatcctccggcagagttttcggctacaaagtttgcttcattcatcacccagtactccacaggacaagtgagcgtggaaattgaatgggagctgcagaaagaaaacagtaagcgctggaatcctgaagtgcagtacacctccaactacgcgaaatctgccaacgttgatttcactgtggacaacaatggactttatactgagcctcgccccattggcacccgttaccttacccgtcccctttaa

SEQ ID NO: 23 - DNA, 1611 bp, Adeno-associated virus
  source 1..1611: mol_type = genomic DNA
  CDS 1..1611: gene = AAVrh92 capsid protein VP3
atgcgtgcagcaccgggcggaaatgctgtcgatgcgggacaaggttccgatggagtgggtaatgcctcgggtgattggcattgcgattccacctggtctgagggcaaggtcacaacaacctcgaccagaacctgggtcttgcccacctacaacaaccacctgtacctgcggctcggaacaacatcaaacagcaacacctacaacggattctccaccccctggggatactttgactttaacagattccactgtcacttctcaccacgtgactggcaaagactcatcaacaacaactggggactacgaccaaaagccatgcgcgttaaaatcttcaatatccaagttaaggaggtcacaacgtcgaacggcgagactacggtcgctaataaccttaccagcacggttcagatatttgcggactcgtcgtatgagctcccgtacgtgatggacgctggacaagaggggagcctgcctcctttccccaatgacgtctttatggtgcctcaatatggctactgtggcatcgtgactggcgagaatcagaaccagacggacagaaatgccttctactgcctggagtattttccttcacaaatgttgagaactggcaacaactttgaaatggcttacaactttgagaaggtgccgttccactcaatgtatgctcacagccagagcctggacagactgatgaatcccctcctggaccagtacctgtggcacttacagtcgactacctctggagggactctgaatcaaggcaatgcagcaaccacatttggaaaaatcaggagtggagactttgccttttacagaaagaactggctgcctgggccttgtgttaagcagcagagattctcaaagactgccagtcaaaactacaagattcctgccagcgggggcgacgctctgttaaagtatgacacccactataccttaaacaaccggtggagcaacatcgcgcccggacctccaatggccacagccggaccttcggatggggacttcagtaacgcccagcttatattccctggaccatctgttaccggaaatacaacaacttcagccaacaatctgttgtttacatcagaagaagaaattgctgccaccaacccaagagacacggacatgtttggccagattgctgacaataatcagaatgctacaactgctcccataaccggcaacgtgactgctatgggagtgctgcctggcatggtgtggcaaaacagagacatttactaccaagggccaatttgggccaagatcccacacgcggacggacattttcatccttcaccgctgattggtgggtttggactgaaacacccgcctccccagatattcatcaagaacactcccgtacctgccaatcctgcgacaaccttcactgcagccagagtggactctttcattacacaatacagcaccggccaggtcgctgttcagattgaatgggaaattgaaaaggaacgctccaaacgctggaatcctgaagtgcagtttacttcaaactatgggaaccagtcttctatgttgtgggctcctgatacaactgggaagtatacagagccgcgggttattggctctcgttatttgactaatcatttg

SEQ ID NO: 24 - DNA, 1569 bp, Adeno-associated virus
  source 1..1569: mol_type = genomic DNA
  CDS 1..1566: gene = AAVrh93 capsid protein VP3
atggcagacaataacgaaggtgccgacggagtgggtaatgcctcaggaaattggcattgcgattccacatggctgggcgacagagtcatcaccaccagcacccgaacctgggccctgcccacctacaacaaccacctctacaagcaaatctccagtcagagcggggctaccaacgacaaccacttctttggctacagcaccccctgggggtattttgacttcaacagattccactgccacttctcaccacgtgactggcagcgactcatcaacaacaactggggattccggcccaagaagctgcggttcaagctcttcaacatccaagtcaaagaggttacgacgaacgacggcgtcacaaccatcgctaataaccttaccagcacgattcaggttttctcggactcggagtaccagctgccgtacgtcctcggctctgcgcaccagggctgcctccctccgttcccggcggacgtcttcatgattcctcagtacggctacctgacgctcaacaacggtagtcagtctgtgggtcgttcttccttctactgcctggaatatttcccctctcaaatgctgagaacgggcaacaactttgaattcagttacaccttcgaggatgtgcctttccacagcagctacgcgcatagccagagcctggaccggctgatgaatcctcttatcgaccagtatctgtactacctggcccggacccagagcaccacgggttccaccagagagctgcagtttcatcaggctgggcccaacactatggccgagcaatcaaagaactggttacctggtccttgcttccggcaacaacgcgtttccaaggtgctggatcagaacgccaacagcaactttgcctggactgctgccactaaatatcacttgaatgggcgtaactctctgaccaatccgggagttcccatggcaacacacaaggacgacgaggacaggttctttcccattaacggggtgctggtttttggcaagaccggagccgccaacaaaacaacgctggaaaatgttctgatgacaaacgaagaagaaatcaaaactaccaacccggtggctacagaagaatatggcgtggtttccagcaatttgcaggctgggactacaaacccacagacactgactgttaacaaccagggggccttacctggcatggtgtggcagaacagggacgtgtacctgcaaggtcccatctgggccaaaattcctcacacggacggcaactttcacccgtctcctttgatgggtggatttggacttaaacacccgcctcctcagatcctaattaaaaacacgcctgttcctgccaatcctccggaggtgtttactcctgccaagtttgcttcgttcatcacgcagtacagcaccggtcaggtcagcgtggaaatcgagtgggagctgcagaaagaaaacagcaagcgctggaacccagagattcagtacacttccaattttgataaatctaataatgtggactttgctgttaacaatgaaggtgtttactctgagcctcgccccattggcactcgctacctcacccgtaacctgtaa

SEQ ID NO: 25 - DNA, 1605 bp, Adeno-associated virus
  source 1..1605: mol_type = genomic DNA
  CDS 1..1602: gene = AAVrh94 capsid protein VP3
atggctgcaggcggtggcgcaccaatggcagacaataacgaaggtgccgacggagtgggtaatgcctcaggaaattggcattgcgattccacatggctgggcgacagagtcatcaccaccagcacccgaacctgggcccttcccacctacaacaaccacctctacaagcaaatctccagtgaaactgcaggtagtaccaacgacaacacctacttcggctacagcaccccctgggggtattttgacttcaacagattccactgccacttctcaccacgtgactggcagcgactcatcaacaacaactggggattccggcccaagaagctcaacttcaagctcttcaacatccaggtcaaggaggtcacgacgaatgacggcgtcacgaccatcgctaataaccttaccagcacggttcaggtattctcggactcggagtaccagctgccgtacgtcctcggctctgcgcaccagggctgtttgcctccgttcccggcggacgtcttcatgattcctcagtacggctacctgactctcaacaatggcagccaatcggtgggacgttcttccttctactgcctggaatacttcccctctcagatgctgagaacgggtaacaacttcaccttcagctacacctttgaggacgtgcctttccacagcagctacgcgcacagccagagcctggaccggctgatgaatcccctcatcgaccagtacctgtactacctggctagaacacagagtaacccaggaggcacatctggcaatcgggaactgcagttttaccagggcggcccttctactatggccgaacaagccaagaactggttacctggaccctgcttccggcaacagagagtgtccaaaacgctggatcaaaacaacaacagcaactttgcttggactggtgccaccaagtatcacctgaacggcagaaactcattggttaatcctggtgtcgccatggcaactcacaaggacgacgaggaccgatttttcccatccagcggagtcctgatttttggaaaaactggagcaaccaacaaaactacattggaaaacgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtatctgcagggtcccatctgggccaagattcctcacacggatggcaactttcacccgtctcctctcatgggcggctttggacttaaacatccgcctcctcagattctcatcaagaacactcccgttcctgctaatcctccggaggtgtttactcctgccaagtttgcttcgtttatcacgcagtacagcaccggacaagtcagcgtggagatcgagtgggagctgcagaaggagaacagcaagcgctggaatccagagattcagtatacctccaactttgagaaacagactggtgtggactttgctgttgacagccagggtatttactctgagcctcgccccattggcacccgttacctcacccgtaatctgtaa

SEQ ID NO: 26 - DNA, 1581 bp, Adeno-associated virus
  source 1..1581: mol_type = genomic DNA
  CDS 1..1578: gene = AAVrh95 capsid protein VP3
atggcagacaataacgaaggtgccgacggagtgggtaatgcctcaggaaattggcattgcgattccacatggctgggcgacagagtcatcaccaccagcacccgaacctgggccctgcccacctacaacaaccacctctacaagcaaatctccagtgaaactgcaggtagcaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggagtaccagctgccgtacgtcctcggctctgcgcaccagggctgcctgcctccgttcccggcggacgtcttcatgattcctcagtacggctacctgacgctcaacaatggcagtcagtctgtgggacgttcctccttctattgcctggagtacttcccttctcagatgctgagaacgggcaacaacttcgagttcagctacagcttcgaggacgtgcctttccacagcagctacgcgcacagccagagcctggaccggctgatgaaccctctcatcgaccagtacctgtactacctggccagaacacagagtaacccaggaggcacagctggcactcgggaactgcagttttaccagggcgggccttccaccatggctgaacaagccaagaactggttacccggaccttgcttccggcaacaaagagtctccaaaacgctggatcaaaataacaacagcaactttgcttggactggtgccaccaaatatcacctgaacggcagaaactcattggttaatcctggtgtcgccatggcaactcacaaggacgacgaggaccgctttttcccatccagcggagtactgatttttggaaaaactggagcaactaacaaaactacattggaaaacgtgttaatgacaaatgaagaagaaattcgtcctactaatcctgtggccacagaagaatacgggatagtcagcagcaacttacaagcggccaatactgcagcccagacacaagttgtcaacaaccagggagccttacctggcatggtttggcagaaccgggatgtgtacttgcagggtcccatctgggccaagattcctcacacggacggcaactttcacccgtctcctctcatgggcggctttggactgaagcatccgcctcctcagatcctgatcaagaatacacccgtacctgctaatcctccggaggtatttactcctgccaagtttgcttctttcatcacacagtacagcaccggacaagtcagcgtggagatcgagtgggagctgcagaaggagaacagcaagcgctggaacccggagattcagtacacctccaactttgaaaagcagactggtgtggactttgcagttgacagccagggtgtttactctgagcctcgccccattggcactcgttacctcacccgtaacctgtaa

SEQ ID NO: 27 - DNA, 1608 bp, Adeno-associated virus
  source 1..1608: mol_type = genomic DNA
  CDS 1..1605: gene = AAVrh97 capsid protein VP3
atggctgcaggcggtggcgcaccaatggcagacaataacgaaggtgccgacggagtgggtagttcctcgggaaattggcattgcgattccacatggctgggcgacagagtcatcaccaccagcacccgaacctgggccctgcccacctacaacaaccacctctacaagcaaatctccaacgggacctcgggaggcagcaccaacgacaacacctacttcggctacagcaccccctgggggtattttgactttaacagattccactgccacttctcaccacgtgactggcagcgactcatcaacaacaactggggattccggcccaagagactcagcttcaagctcttcaacatccaggtcaaggaggttacgcagaatgaaggcaccaagaccatcgccaataacctcaccagcaccatccaggtgtttacggactcggaataccagctgccgtacgtcctcggctctgcccaccagggctgcctgcctccgttcccggcggacgtcttcatgattcctcagtacggctacctgactctcaacaatggcagccaggccgtgggacgttcctccttctactgcctggagtacttcccctctcagatgctgagaacgggcaacaacttctccttcagctacaccttcgaagacgtgcctttccacagcagctacgcgcacagccagagcctggaccggctgatgaatcccctcatcgaccaatacctgtactacctgtcaagaacccagtctacgggcggtacagcgggaacgcagcagttgctgttttctcaggccgggcctagcaacatgtcggctcaggccagaaactggctgccaggaccctgctacagacagcagcgagtctccacgacactgtcgcaaaacaacaacagcaactttgcttggaccggtgccaccaaatatcacctgaacggcagagactctctggtgaatccgggcgtagccatggcaaccaacaaggacgacgaggaccgcttcttcccatctagcggcatcctcatgtttggcaagcagggagctggaaaagacaacgtggactatagcaacgtgatgctcaccagcgaggaagagatcaagaccaccaaccccgtggccacagaacagtatggcgtggtggctgataacctgcagcagcaaaacaccgctcctattgtaggggccgtcaacagccagggagccttacctggtatggtctggcagaaccgggacgtatacctgcagggtccaatctgggccaagattcctcacacggatggcaactttcacccgtctcctctcatgggcggctttggacttaaacatccgcctcctcagatcctcatcaagaacactcctgttcctgcggaccctccaacggcgttcaaccaggccaagctgaattctttcatcacgcagtacagcaccggacaagtcagcgtggagattgagtgggagctgcagaaggaaaacagcaagcgctggaacccagagattcagtatacctccaactactacaaatctacaaatgtggactttgctgtcaatacagagggtgtttattctgagcctcgccccattggcactcgttacctcacccgtaatctgtaa

SEQ ID NO: 28 - DNA, 1608 bp, Adeno-associated virus
  source 1..1608: mol_type = genomic DNA
  CDS 1..1605: gene = AAVrh98 capsid protein VP3
atggctgcaggcggtggcgcaccaatggcagacaataacgaaggtgccgacggagtgggtagttcctcgggaaattggcattgcgattccacatggctgggcgacagagtcatcaccaccagcacccgaacctgggccctgcccacctacaacaaccacctctacaagcaaatctccaacgggacctcgggaggcagcaccaacgacaacacctacttcggctacagcaccccctgggggtattttgactttaacagattccactgccacttctcaccacgtgactggcagcgactcatcaacaacaactggggattccggcccaagagactcagcttcaagctcttcaacatccaggtcaaggaggttacgcagaatgaaggcaccaagaccatcgccaataacctcaccagcaccatccaggtgtttacggactcggaataccagctgccgtacgtcctcggctctgcccaccagggctgcctgcctccgttcccggcggacgtcttcatgattcctcagtacggctacctgactctcaacaatggcagccaggccgtgggacgttcctccttctactgcctggagtacttcccctctcagatgctgagaacgggcaacaacttctccttcagctacaccttcgaagacgtgcctttccacagcagctacgcgcacagccagagcctggaccggctgatgaatcccctcatcgaccaatacctgtactacctgtcaagaacccagtctacgggcggtacagcgggaacgcagcagttgctgttttctcaggccgggcctagcaacatgtctgctcaggccaaaaactggctgccaggactctgctacagacagcagcgagtctccacgacactgtcgcaaaacaacaacagcaactttgcttggaccggtgccaccaaatatcacctgaacggcagagactctctggtgaatccgggcgtagccatggcaaccaacaaggacgacgaggaccgcttcttcccatccagcggcatcctcatgtttggcaagcagggagctggaaaagacaacgtggactatagcaacgtgatgctcaccagcgaggaagagatcaagaccaccaaccccgtggccacagaacagtatggcgtggtggctgataacctgcagcagcaaaacaccgctcctattgtaggggccgtcaacagccagggagccttacctggtatggtctggcagaaccgggacgtatacctgcagggtccaatctgggccaagattcctcacacggatggcaactttcacccgtctcctctcatgggcggctttggacttaaacatccgcctcctcagatcctcatcaagaacactcctgttcctgcggaccctccaacggcgttcaaccaggccaagctgaattctttcatcacgcagtacagcaccggacaagtcagcgtggagattgagtgggagctgcagaaggaaaacagcaagcgctggaacccagagattcagtatacctccaactactacaaatctacaaatgtggactttgctgtcaatacagagggtgtttattctgagcctcgccccattggcactcgttacctcacccgtaatctgtaa

SEQ ID NO: 29 - DNA, 1587 bp, Adeno-associated virus
  source 1..1587: mol_type = genomic DNA
  CDS 1..1584: gene = AAVrh99 capsid protein VP3
atggcagacaataacgaaggtgccgacggagtgggtagttcctcgggaaattggcattgcgattccacatggctgggcgacagagtcatcaccaccagcacccgaacctgggccctgcccacctacaacaaccacctctacaagcaaatctccaacgggacctcgggaggcagcaccaacgacaacacctacttcggctacagcaccccctgggggtattttgactttaacagattccactgccacttctcaccacgtgactggcagcgacttatcaacaacaactggggattccggcccaagaagctgcggttcaagctcttcaacatccaggtcaaggaggtcacgacgaatgacggcgttacgaccatcgctaataaccttaccagcacgattcaggtattctcggactcggagtaccagctgccgtacgtcctcggctctgcgcaccagggctgcctgcctccgttcccggcggacgtcttcatgattcctcagtatggctacctgacgctcaacaacggaagtcagtctgtgggacgttcctccttctattgcctggagtacttcccttctcagatgctgagaacgggcaacaacttcgagttcagctacagcttcgaggacgtgcctttccacagcagctacgcgcacagccagagcctggaccggctgatgaaccctctcatcgaccagtacctgtactacctggccagaacacagagtaacccaggaggcacagctggcactcgggaactgcagttttaccagggcgggccttccaccatgtctgaacaagccaaaaactggctacccggaccttgttatcggcagcaacgtgtttccacgacactgtcgcaaaacaacaacagcaactttgcctggaccggtgccaccaaataccacctgaacggaagagactctctggtaaatccgggtgtcgccatggcaaccaacaaggacgacgaggaccgcttcttcccatccagcggcatcctcatgtttggcaagcagggggctggaaaagacaacgtggactatagcaacgtgatgctaaccagcgaggaagaaatcaagaccactaaccctgtggccacagaacagtatggtgtggtggcggataacctgcagcagcaaaacacagctcctattgtgggggccgtcaacagccaaggagccttacctggcatggtttggcagaaccgggacgtgtacctgcagggtcccatctgggccaagattcctcacacggatggtaactttcacccgtctcctctcatgggcggctttggacttaaacatccgcctcctcagatcctgatcaagaacactcccgttcctgcggatcctccaacggcgttcaaccaggccaagctgaactctttcatcacgcagtacagcaccggacaagtcagcgtggagatcgagtgggagctgcagaaggagaacagcaagcgctggaacccagagattcagtatacctccaactactacaaatctacaaatgtggactttgctgttaatactgagggtgtttactctgagcctcgccccattggcacccgttacctcacccgtaatctgtaa

SEQ ID NO: 30 - protein, 535 aa, Adeno-associated virus
  REGION 1..535: note = AAVrh90 capsid protein VP3
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 31 - protein, 534 aa, Adeno-associated virus
  REGION 1..534: note = AAVrh91 capsid protein VP3
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKENAGASNTALDNVMITDEEEIKATNPVATERFGTVAVNLQSSNTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 32 - protein, 537 aa, Adeno-associated virus
  REGION 1..537: note = AAVrh92 capsid protein VP3
MRAAPGGNAVDAGQGSDGVGNASGDWHCDSTWSEGKVTTTSTRTWVLPTYNNHLYLRLGTTSNSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGGTLNQGNAATTFGKIRSGDFAFYRKNWLPGPCVKQQRFSKTASQNYKIPASGGDALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 33 - protein, 522 aa, Adeno-associated virus
  REGION 1..522: note = AAVrh93 capsid protein VP3
MADNNEGADGVGNASGNWHCDSTWLGDRVITTSTRTWALPTYNNHLYKQISSQSGATNDNHFFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYTFEDVPFHSSYAHSQSLDRLMNPLIDQYLYYLARTQSTTGSTRELQFHQAGPNTMAEQSKNWLPGPCFRQQRVSKVLDQNANSNFAWTAATKYHLNGRNSLTNPGVPMATHKDDEDRFFPINGVLVFGKTGAANKTTLENVLMTNEEEIKTTNPVATEEYGVVSSNLQAGTTNPQTLTVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFDKSNNVDFAVNNEGVYSEPRPIGTRYLTRNL

SEQ ID NO: 34 - protein, 534 aa, Adeno-associated virus
  REGION 1..534: note = AAVrh94 capsid protein VP3
MAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLNFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLARTQSNPGGTSGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGIYSEPRPIGTRYLTRNL

SEQ ID NO: 35 - protein, 526 aa, Adeno-associated virus
  REGION 1..526: note = AAVrh95 capsid protein VP3
MADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGTRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 36 - protein, 535 aa, Adeno-associated virus
  REGION 1..535: note = AAVrh97 capsid protein VP3
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 37 - protein, 535 aa, Adeno-associated virus
  REGION 1..535: note = AAVrh98 capsid protein VP3
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQAKNWLPGLCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 38 - protein, 528 aa, Adeno-associated virus
  REGION 1..528: note = AAVrh99 capsid protein VP3
MADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGTRELQFYQGGPSTMSEQAKNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 39 - DNA, 26 bp, synthetic construct
  source 1..26: mol_type = other DNA
gctgcgtcaactggaccaatgagaac

SEQ ID NO: 40 - DNA, 28 bp, synthetic construct
  source 1..28: mol_type = other DNA
cgcagagaccaaagttcaactgaaacga

SEQ ID NO: 41 - protein, 738 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIVWELQKENSKRWNPEIQYTSNYYKSTNVDFAVNTEGVYSEPRPIGTRYLTRNL

SEQ ID NO: 42 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 43 - protein, 733 aa, Adeno-associated virus
MAADGYLPDWLEDNLSEGIREWWDLKPGAPKPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 44 - protein, 728 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPIDSPDSSTGIGKKGQQPAKKKLNFGQTGDSESVPDPQPLSEPPAAPTGVGSGTVAAGGGAPMADNNEGADGVGNASGNWHCDSTWLGDRVITTSTRTWALPTYNNHLYKQISSQSGATNDNHFFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQAVGRSSFYCLEYFPSQMLRTGNNFEFSYTFEDVPFHSSYAHSQSLDRVMNPLIDQYLYYLARTQSTTGSTRELQFHQAGPNTMAEQSKNWLPGPCFRQQRVSKVLDQNANSNFAWTAATKYHLNGRNSLTNPGVPMATHKDDEDRFFPINGVLVFGKTGAANKTTLENVLMTNEEEIKTTNPVATEEYGVVSSNLQAGTTNPQTLTVNNQGALPGMVWQNRDVYLQGPIWAKIPQTDGNFHPSPLMGGFGLKHPPPQILIKNTPVPANPPEVFTPAKFASFITQYSTGQVSVEIEWELQKENSKRWNPEIQYTSNFDKSNNVDFAVNNEGVYSEPRPIGTRYLTRNL

SEQ ID NO: 45 - protein, 737 aa, Adeno-associated virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLNFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLARTQSNPGGTSGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGIYSEPRPIGTRYLTRNL

SEQ ID NO: 46 - protein, 737 aa, Adeno-associated virus
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 47 - protein, 738 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKATNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 48 - protein, 535 aa, Adeno-associated virus
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIVWELQKENSKRWNPEIQYTSNYYKSTNVDFAVNTEGVYSEPRPIGTRYLTRNL

SEQ ID NO: 49 - protein, 534 aa, Adeno-associated virus
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 50 - protein, 537 aa, Adeno-associated virus
MRAAPGGNAVDAGQGSDGVGNASGDWHCDSTWSEGKVTTTSTRTWVLPTYNNHLYLRLGTTSNSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 51 - protein, 522 aa, Adeno-associated virus
MADNNEGADGVGNASGNWHCDSTWLGDRVITTSTRTWALPTYNNHLYKQISSQSGATNDNHFFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQAVGRSSFYCLEYFPSQMLRTGNNFEFSYTFEDVPFHSSYAHSQSLDRVMNPLIDQYLYYLARTQSTTGSTRELQFHQAGPNTMAEQSKNWLPGPCFRQQRVSKVLDQNANSNFAWTAATKYHLNGRNSLTNPGVPMATHKDDEDRFFPINGVLVFGKTGAANKTTLENVLMTNEEEIKTTNPVATEEYGVVSSNLQAGTTNPQTLTVNNQGALPGMVWQNRDVYLQGPIWAKIPQTDGNFHPSPLMGGFGLKHPPPQILIKNTPVPANPPEVFTPAKFASFITQYSTGQVSVEIEWELQKENSKRWNPEIQYTSNFDKSNNVDFAVNNEGVYSEPRPIGTRYLTRNL

SEQ ID NO: 52 - protein, 534 aa, Adeno-associated virus; identical to SEQ ID NO: 34

SEQ ID NO: 53 - protein, 526 aa, Adeno-associated virus
MADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 54 - protein, 527 aa, Adeno-associated virus
MADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTAGTQQLLFSQAGPSNMSAQAKNWLPGPCYRQQRVSTTLSQNNNSNFAWTGATKYHLNGRDSLVNPGVAMATHKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTTFNQAKLNSFITQYSTGQVSVEIEWELQKENSKRWNPEIQYTSNYYKSTNVDFAVNTEGVYSEPRPIGTRYLTRNL